(19) United States
(12) Patent Application Publication
(10) Pub. No.: US 2021/0017514 A1
(43) Pub. Date: Jan. 21, 2021

(54) METHODS FOR CONTROLLING GENE EXPRESSION

(71) Applicant: THE CHANCELLOR, MASTERS AND SCHOLARS OF THE UNIVERSITY OF CAMBRIDGE, Cambridgeshire (GB)

(72) Inventors: Alexander Morgan Jones, Cambridgeshire (GB); Bo Larsen, Cambridgeshire (GB)

(21) Appl. No.: 16/977,394

(22) PCT Filed: Mar. 1, 2019
(86) PCT No.: PCT/GB2019/050582

§ 371 Date: Sep. 1, 2020

(30) Foreign Application Priority Data
Mar. 2, 2018 (GB) 1803398.5

(51) Int. Cl. C12N 15/10; C12N 15/85; C12Q 1/6876; C12N 15/67

(52) U.S. Cl. CPC C12N 15/10; C12N 15/8509; C12Q 2525/143; C12N 15/67; C12Q 1/6876

(57) ABSTRACT

The invention relates to methods for precisely controlling expressions of a target gene in an organism using a light-inducible kinase and a response regulator. The invention also relates to nucleic acid constructs and nucleic acids encoding the light-inducible kinase and response regulator, as well as organisms expressing these constructs.

### FIELD OF THE INVENTION

The invention relates to methods for precisely controlling expression levels of a nucleic acid sequence, such as a target gene, in an organism using a light-inducible kinase and a response regulator. The invention also relates to nucleic acid constructs and nucleic acids encoding the light-inducible kinase and response regulator, as well as organisms expressing these constructs.

### BACKGROUND OF THE INVENTION

There are thousands of genes in cells which are regulated to orchestrate developmental processes and physiological activities. Some gene functions are unknown in certain contexts, and some are well defined and are of interest to manipulate to produce an advantageous effect. It is therefore of growing interest to be able to selectively regulate gene expression. In research, it is an important tool to probe the function of genes and/or processes controlled by genes, including developmental processes or biochemical activities. In the case of plants, it is of particular interest to manipulate genes relating to physiological processes such as flowering or germination, or pest resistance, for commercial and agroeconomic purposes.

Current systems for genetic manipulation, including inducing or repressing expression of genes, mainly rely on small-molecule inducers such as doxycycline (Motta-Mena et al, 2014, Nature Chemical Biology). These chemical inducers are associated with a number of disadvantages, as the chemical can be pharmacologically active and therefore have off-target effects, is often limited by diffusion into tissue, cannot be localised into small areas or removed after application, and can be toxic to both the target organism, people, and the environment.

More recently, the field of “optogenetics” for the regulation of gene expression has grown. These optogenetic systems allow for gene expression to be selectively controlled by exposure to minimally invasive light stimuli in a highly selective spatiotemporal manner. This technique circumvents the previously described problems of chemically inducible systems. In addition, light stimuli are cheap to generate, environmentally benign and can potentially be applied repeatedly over large areas and over long periods, which may be particularly advantageous in crop plants, or light stimuli can be applied with incredible resolution using lasers. There have been some optogenetic systems described, however these are accompanied with a number of limitations or issues, including; low transcriptional activation, long deactivation times, use of exotic chromophores not found endogenously, potential interference with endogenous signalling pathways and the need for multiple protein components (Motta-Mena et al, 2014). It is well known in the field that there are many biological challenges associated with optogenetic systems, including the development of appropriate light-sensitive proteins (Hunter, 2016, EMBO reports). In particular, the application of optogenetic tools in plants presents further difficulties in that plants require light for growth and development, and thus far only a red/far-red light inducible “on/off” system has been applied to plants (Ochoa-Fernandez et al. 2016, Methods in Molecular Biology).

The present invention addresses the need for an improved optogenetic system that can be used in any organism, including plants.

### SUMMARY OF THE INVENTION

We have created a new tool for manipulating gene expression with light, named the “Highlighter system”. This system repurposes a photoreversible two-component signal transduction system termed CcaS-CcaR, originally derived from a native cyanobacterium Synechocystis sp. PCC6803, for use in cells and whole organisms, including plants. In nature, cyanobacteria use this system to change the composition of their light-harvesting pigments in response to green and red light for photosynthetic purposes or for resistance to photodamage (Hirose et al, 2010, PNAS., Abe et al, 2014, Microbial Biotechnology). When cyanobacteria are exposed to green light for example, CcaS is activated by a chromophore-dependent, light-induced conformational change, and phosphorylates CcaR which then induces CcaR binding to a promoter region that drives transcription of the transcriptional regulator for regulating the synthesis of the light-harvesting pigment phycoerythrin.

This invention harnesses this natural phenomenon and functions, in its most simple form, by expressing in a target cell or organism, a CcaS variant (in this invention known as the light-responsive histidine kinase (LRHK)) and a CcaR variant (in this invention known as the response regulator (RR)) along with a target gene of interest that is under the control of a response-regulator specific promoter. In this way, expression of the target gene is controlled as when the LRHK is exposed to an activating wavelength of light, it phosphorylates the RR which can then bind to its cognate promoter to drive transcription of the target gene. A strong advantage of the CcaS-CcaR system is that the components of the CcaS-CcaR system are not present in plants, so therefore the system is orthogonal to plant signalling pathways, and therefore will less likely interfere with, or be interfered by, endogenous signalling pathways. This system has been used in cyanobacteria and E. coli to drive target gene expression upon green-light stimulation (Abe et al, 2014; Tabor et al, 2011). However, we have further altered this system, wherein the system can be activated with a range of different light wavelengths, with a view to utilising the system in plants in particular through a number of modifications.

These improvements include modification to CcaS (codon optimisation, improved photoswitching with the PΦB chromophore present in plants, untethering of CcaS from the cell membrane and addition of a nuclear localisation signal) and to CcaR (codon optimisation, addition of a C-terminal nuclear localisation signal, addition of a eukaryotic transactivation domain). We have also created a plant vector expression system to deliver the system to plants that includes a synthetic promoter, whose activity level can be modulated via the response regulator, and optionally a fluorescent output reader for normalisation purposes, and ribosomal skipping sequences to reduce vector size. The system is designed to exhibit one target gene expression state during plant growth in normal light-dark cycles, and an altered target gene expression state following treatment with light spectra that are not found in horticultural environment.

There are many possible applications of this system, whereby gene expression can be precisely and effectively manipulated to study a range of biological processes, or induce advantageous properties in an organism. The system can be used in a precise manner, both spatially and temporally, to for example, target a certain area of the plant such as the leaves, or for example, at a defined time to trigger a biological process such as the timing of flowering or germination. This would allow for specific interventions for improved agronomic outcomes.

The invention described here is thus aimed at providing light-regulated gene expression in cells and organisms and related methods, thus providing products and methods of research and agricultural importance.

In one aspect of the invention, there is provided a nucleic acid construct comprising a nucleic acid encoding a light-responsive histidine kinase and/or a nucleic acid encoding a response regulator, wherein the nucleic acid encodes a light-responsive histidine kinase as defined in any one of SEQ ID NOs: 1, 3, 5, 7, 9 or 11 or a functional variant thereof and wherein the response regulator encodes a response regulator as defined in any of SEQ ID NOs 13 or 15 or a functional variant thereof.

In one embodiment, the nucleic acid encoding a light-responsive histidine kinase comprises or consists of SEQ ID NO 2, 4, 6, 8, 10 or 12 or a functional variant thereof or comprises or consists of SEQ ID NO: 47, 48, 49 or 50 or a functional variant thereof.

In another embodiment, the nucleic acid encoding a response regulator comprises or consists of SEQ ID NO: 14 or 16 or a functional variant thereof.

In a further embodiment, the construct comprises at least one regulatory sequence operably linked to at least one of the light-responsive histidine kinase and the response regulator. Preferably, the regulatory sequence is operably linked to the light-responsive histidine kinase and the response regulator.

In another embodiment, the construct further comprises a reporter sequence. Preferably, the reporter sequence is operably linked to a regulatory sequence. More preferably, the light-responsive histidine kinase, the response regulator and the reporter sequence are operably linked to a single regulatory sequence.

In a further embodiment, the construct further comprises at least one terminator sequence operably linked to at least one, preferably at least two, more preferably all three of the light-responsive histidine kinase, the response regulator and the reporter sequence.

In one embodiment, the regulatory sequence is a constitutive promoter. For example, the promoter is the UBQ10 promoter or a functional variant thereof.

In a further embodiment, the construct further comprises a target sequence operably linked to a regulatory sequence that is specifically activated by the response regulator. In one embodiment, the regulatory sequence comprises a nucleic acid sequence as defined in SEQ ID NO: 17 or a functional variant thereof. In a further embodiment, the target sequence is operably linked to a terminator sequence.

In another aspect of the invention, there is provided a vector, preferably an expression vector, comprising the nucleic acid construct as described herein.

In a further aspect of the invention, there is provided a host cell comprising a nucleic acid construct as described herein or a vector as described herein. Preferably, the cell is a eukaryotic or prokaryotic cell. More preferably, the eukaryotic cell is a plant cell.

In another aspect of the invention, there is provided a transgenic organism expressing the nucleic acid construct as described herein or a vector as described herein. In a preferred embodiment, the organism is a plant.

In another aspect of the invention, there is provided a method of producing a transgenic organism as described herein, the method comprising:
a. selecting a part of the organism;
b. transfecting at least one cell of the part of the organism of part (a) with the nucleic acid construct as described herein or the vector as described herein; and
c. regenerating at least one organism derived from the transfected cell or cells.

In a further aspect, there is provided an organism obtained or obtainable by the method described herein. Preferably, the organism is a plant.

In another aspect of the invention, there is provided a method of modulating expression of a target gene in an organism, the method comprising introducing and expressing a nucleic acid construct as described herein or a vector as described herein in said organism and applying at least one wavelength of light. In one embodiment, the wavelength of light activates or represses activation of a LRHK

In a further aspect of the invention, there is also provided a method of modulating any biochemical response in an organism, the method comprising introducing and expressing at least one nucleic acid construct as described herein or a vector as described herein in said organism and applying at least one wavelength of light. In one embodiment, the biochemical response is a developmental process or physiological response. Preferably, the biochemical response is modulated by modulating expression of at least one target gene. In one embodiment, the wavelength of light activates or represses activation of a LRHK.

The wavelength of light may be referred to as an activating or repressing wavelength.

In one embodiment, the wavelength of light may have one of the following ranges, 370-400 (ultraviolet light), 430 to 495 nm (blue light), 495 to 570 nm (green light), 570 nm to 600 nm (yellow/orange light), 600 to 750 nm (red light) or far-red (750 to 850 nm), or be a white light (as described below). In another embodiment, the wavelength of light may be dark light (as described below). In a further embodiment, the wavelength of light may be white light enriched with at least one of red, blue or green light.

In one embodiment, expression of a target gene can be increased or decreased by applying at least one first wavelength of light.

In a further embodiment, expression of a target gene can be decreased or further increased by applying at least one second wavelength of light, wherein the first wavelength of light is different from the second wavelength of light.

In one embodiment, the first wavelength of light that increases expression of the target gene is preferably green, white, dark or red light or is white light enriched with red light.

In another embodiment, the first wavelength of light that decreases expression of the target gene is preferably blue light or is white light enriched with blue light.

In a further embodiment, the second wavelength of light that further increases expression of a target gene is red light. In this embodiment, the first wavelength of light is preferably white, green or dark light.

In another embodiment, the second wavelength of light that decreases expression of a target gene is blue light. In this embodiment, the first wavelength may be red, green, white or dark light.

In another embodiment, the first wavelength of light may be blue light and the second wavelength of light red light or vice versa.

In another aspect of the invention, there is provided a photoreceptor molecule comprising a phytochrome and a chromophore, wherein the phytochrome comprises an amino acid sequence as defined in any of SEQ ID NOs 1, 3, 5, 7, 9 and 11 or a variant thereof. Preferably, the chromophore is selected from PCB (phycocyanobilin), PφB (phytochromobilin) and BV (biliverdin). More preferably, the chromophore is PφB.

In a further aspect of the invention, there is provided the use of the nucleic acid construct as described above or a vector as described above to modulate expression of a target gene in an organism.

In another aspect of the invention, there is provided the use of the nucleic acid construct as described above or a vector as described above to modulate any biochemical response in an organism, preferably a developmental or physiological response.

In a further aspect of the invention, there is provided a nucleic acid construct comprising a target sequence operably linked to a regulatory sequence, wherein the regulatory sequence is a regulatory sequence that is specifically activated by the response regulator. In one embodiment, the regulatory sequence comprises a nucleic acid sequence as defined in SEQ ID NO: 17 or a functional variant thereof.

In a final aspect of the invention, there is provided a nucleic acid comprising:
a. a nucleic acid sequence encoding a polypeptide as defined in any of SEQ ID NOs 1, 3, 5, 7, 9, 11, 13 and 15;
b. a nucleic acid sequence as defined in any of SEQ ID NOs 2, 4, 6, 8, 10, 12, 14, 16 or 17 or the complementary sequence thereof;
c. a nucleic acid with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the nucleic acid sequence of (a) or (b); or
d. a nucleic acid sequence that is capable of hybridising under stringent conditions as defined herein to the nucleic acid sequence of any of (a) to (c).

### DESCRIPTION OF THE FIGURES

The invention is further described in the following non-limiting figures:

FIG. 1 shows the CcaS-CcaR system repurposed for control of gene expression in E. coli. In darkness, or upon red light illumination, the CcaS-CcaR system remains in/enters its inactive state where sfGFP expression is at its lowest. Upon green light illumination, the kinase activity of CcaS is activated and CcaS phosphorylates and hence activates CcaR (CcaR-P). CcaR-P binds the ccaR CRE, inside the P_{cpcG2-172} promoter sequence and induces sfgfp transcription.

FIG. 2 shows photoswitching Assay in E. coli. Serial dilutions of E. coli cultures expressing the CcaS-CcaR system was grown in 96-well plates On LB media at 37° C., shaking) while receiving light treatments, here blue light (Blue), green light (Blue), red light (Red) and darkness (Dark). The GFP fluorescence was quantified on a fluorimeter, along with the cell density (OD₆₀₀). The Fluorescence was then plotted against the cell density (A). The fluorescence was then estimated at OD600=0.2 and converted into a heat map (B).

FIG. 3 shows chromophore dependency of the CcaS-CcaR system in E. coli. The system was tested under five light regimes; four hour treatments with RGB-white (White), blue, green or red light and in darkness (Dark). CcaS was always coexpressed with CcaR in combination with the biosynthetic machinery to produce PCB, PΦB, BV or no chromophore (Ø). The intensity of green in the heat map corresponds to the level of sfGFP expression observed under the tested conditions.

FIG. 4 shows the A92V mutation enhances CcaS photoswitching with PΦB. CcaS(A92V) with PΦB is repressed by blue light and RGB-white light (White) and activated by green light and red light. CcaS(A92V) behaves like CcaS in the presence of BV and in the absence of chromophores.

FIG. 5 shows bacterial validation of modifications made to CcaS in order for it to function in planta. We simultaneously tested the effects on the photoswitching properties of CcaS of the following modification; the A92 mutation to allow for photoswitching with PΦB, removal of the transmembrane domain (Δ22 or Δ23), and the addition of an N-terminal NLS. The numbers in the table are fluorescence counts in millions.

FIG. 6 shows bacterial testing of the effects of 2A tails on CcaS function.

FIG. 7 shows a schematic of a pHighlighter plant expression vector. The input cassette constitutively expresses a light responsive histidine kinase (LRHK), a reporter (R_{const}) and a response regulator (RR). The constitutive expression of these three proteins from the input cassette is controlled by the UBQ10 promoter (P_{UBQ10}) (SEQ ID NO: 44) and the rbcS terminator (T_{rbcS})(SEQ ID No: 42). The output cassette holds a cognate promoter for the response regulator (P_{RR}), a target gene of interest (Target) and a NOS terminator (T_{NOS})(SEQ ID NO: 43). When the LRHK is exposed to an activating wavelength of light, it phosphorylates the RR, which then binds to its cognate promoter, P_{RR}, and the Target is expressed. The constitutively expressed reporter, R_{const}, allows for the detection of transfected cells during transient transfections of plants and a normalization control if a fluorescent protein is used as Target. LB and RB are the left border and right borders. ColEI and OriV are origins of replication, trfA is a replication initiation protein and Amp^{R} is the bacterial resistance gene against ampicillin.

FIG. 8 shows the cognate promoter, P_{RR}, for the response regulator. The P_{RR} is made up of three ccaR CRE sequences, separated by spacers, and fused to the −51 35S minimal promoter (P_{35Smin(-51)}). +1 denotes the transcription start site (TSS).

FIG. 9 shows ribosomal skipping efficiency in Tobacco. The efficiency of ribosomal skipping for P2A, F2A and F2A₃₀ was tested in transiently transfected tobacco. The graph shows the mean TagRFP signal in the nucleus/mean TagRFP signal in the cytosol. For this experiment, the LRHK, MM:NLS:CcaS(Δ23 A92V), was linked to a downstream TagRFP via the three different 2A sequences, P2A, F2A and F2A₃₀, and expressed from the P_{UBQ}-T_{rbcS} cassette. The controls for perfect ribosomal skipping and complete failure of skipping are TagRFP and NLS:TagRFP. n=4-6, error bars are S.D.

FIG. 10 shows transient expression of the Highlighter system in Tobacco: The plant expression vector, pHighlighter, was transformed into Agrobacterium and used to infiltrate tobacco leaves. The plants were left to express the system for 2 days in the greenhouse and light treated for a minimum of 18 hours.

FIG. 11 shows light-controlled induction of NLS:Venus expression, by four Highlighter system variants, in response to blue light, green light and darkness. The systems were transiently expressed in tobacco as described in FIG. 6. The numbers are YFP mean/RFP mean averages for plant nuclei under the given light condition. ± are S.D., n=3 biological replica (each n is an average of the YFP mean/RFP mean calculated for 15-20 nuclei).

FIG. 12 shows transient expression of the Highlighter system in Tobacco: The plant expression vector, pHighlighter, was transformed into Agrobacterium and used to infiltrate tobacco leaves. The plants were left to express the system for 2 days under continuous blue light conditions and light treated (RGB-white light (White), blue light, green light, red light and darkness) for a minimum of 24 hours.

FIG. 13 shows light-controlled induction of NLS:Venus expression, by four Highlighter system variants, in response to blue light, green light and darkness. The systems were transiently expressed in tobacco as described in FIG. 7. The numbers are YFP mean/RFP mean (specifically NLS:Venus mean signal/NLS:TagRFP mean signal) averages for plant nuclei under the given light condition. The values in the table are the YFP mean/RFP mean average calculated for 22-209 nuclei, ± are 95% confidence intervals.

FIG. 14 shows light-controlled induction of NLS:Venus expression, by three Highlighter system variants. Induction of NLS:Venus expression was measured in response to what the human eye perceives as pure red light (RRR), very red enriched white light (RRW), slightly red enriched white light (RWW, i.e. red light proportion 42% and blue light proportion 32%), slightly blue enriched white light (WWB, i.e. red light proportion 18% and blue light proportion 60%), very blue enriched white light (WBB) and pure blue light (BBB). The systems were transiently expressed in tobacco as shown in FIG. 12. Confocal fluorescence images of tobacco epidermal cells were acquired and IMARIS software was used to segment and quantify fluorescence signals from individual nuclei. The values in the table are mean fluorescence emission values for YFP/RFP calculated for 12-132 nuclei±95% confidence intervals.

FIG. 15 shows quantification of LRHK variants in E. coli. E. coli strains expressing the LRHK variants were quantified after four hour treatments of darkness and eight different light regimes: ultraviolet light (370 nm or 400 nm), blue light (450 nm), green light (520 nm), yellow light (590 nm), orange light (610 nm), red light (630 nm), far red light (700 nm). The LRHKs were coexpressed with CcaR, sfGFP under control of a CcaS/CcaR responsive promoter, and the biosynthetic machinery to produce PΦB. The values are fluorescence counts in millions, corresponding to the level of sfGFP expression observed under the tested light regimes.

FIG. 16 shows conditional complementation of the semi-dwarf phenotype of the ga3ox1-3, ga3ox2-1, nGPS1 Arabidopsis line by using the Highlighter system to control AtGA3OX1 expression levels with blue- and red-enriched white light. (A) The ga3ox1-3, ga3ox2-1, nGPS1 line grown in continuous blue-enriched white light. (B) The ga3ox1-3, ga3ox2-1, nGPS1 line, transformed with the Highlighter system to control GA3OX1 expression levels, grown in continuous blue-enriched white light. (C) The ga3ox1-3, ga3ox2-1, nGPS1 line grown in continuous red-enriched white light. (D) The ga3ox1-3, ga3ox2-1, nGPS1 line, transformed with the Highlighter system to control AtGA3OX1 expression levels, grown in continuous red-enriched white light.

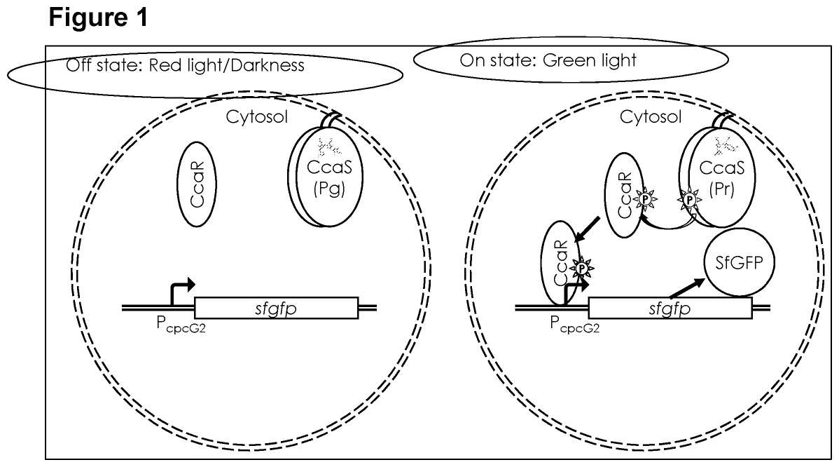

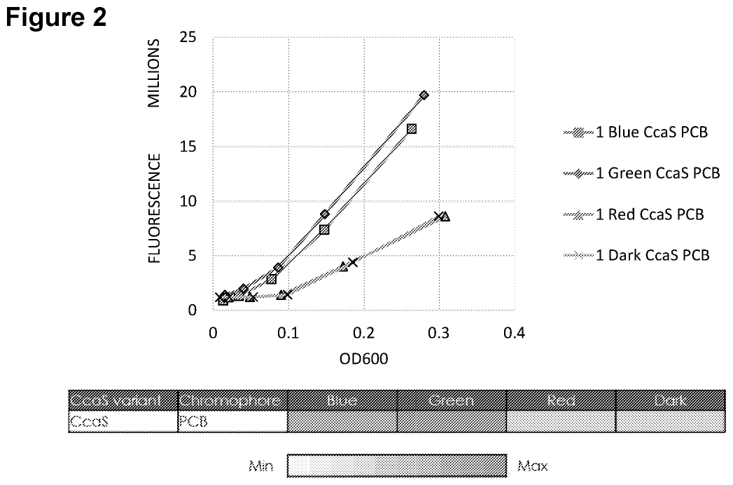

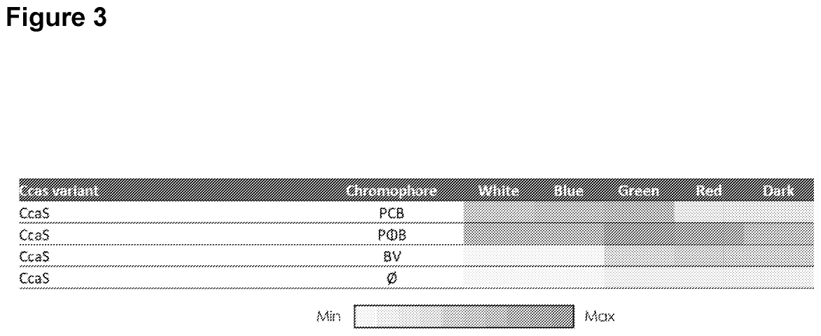

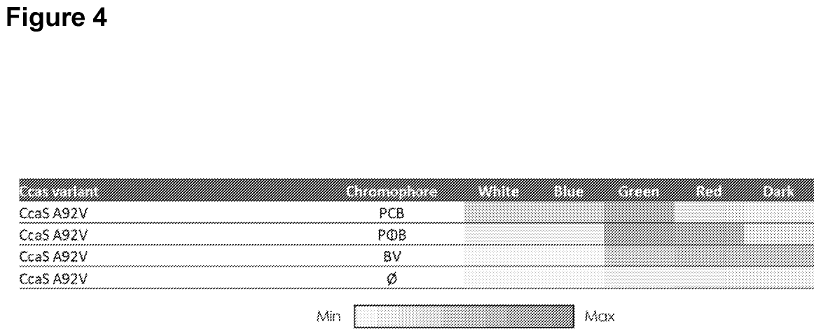

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry, recombinant DNA technology, and bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

In one aspect of the invention, there is provided a nucleic acid construct comprising a light-responsive histidine kinase (LRHK) and/or a response regulator (RR). In a preferred embodiment, the LRHK is a cyanobacteriochrome, more preferably, the cyanobacteriochrome CcaS (complementary chromatic acclimation sensor). In a further preferred embodiment, CcaS comprises a nuclear localisation signal and/or lacks a membrane anchor and/or has a A92V mutation. More preferably, as described above, CcaS comprises or consists of a nucleic acid, wherein the nucleic acid encodes a light-responsive histidine kinase as defined in any one of SEQ ID NOs: 1, 3, 5, 7, 9 or 11 or a functional variant thereof. Preferably, the construct comprises both a LRHK and RR.

In another preferred embodiment, the RR is a transcriptional regulatory protein, preferably a OmpR-class response regulator, and more preferably CcaR (complementary chromatic acclimation regulator). In a preferred embodiment, CcaR comprises a C-terminal nuclear localisation signal and/or a transcription activation or repressor domain, preferably the VP64 eukaryotic transactivation domain. In a particularly preferred embodiment, the response regulator comprises a nucleic acid sequence encoding a response regulator as defined in any of SEQ ID NOs 13 or 15 or a functional variant thereof.

In one embodiment, the nucleic acid encoding a light-responsive histidine kinase comprises or consists of SEQ ID NO 2, 4, 6, 8, 10, 12, 47, 48, 49 or 50 or a functional variant thereof. In a further embodiment, the nucleic acid encoding a response regulator comprises or consists of SEQ ID NO: 14 or 16 or a functional variant thereof.

SEQ ID NOs 1-12 and 47 to 50 relate to exemplary variants of CcaS that may be used in the invention. Similarly, SEQ ID NOs 13-16 relate to exemplary variants of CcaR that may be used in the invention.

### CcaS Variants

SEQ ID NOs 1 and 2 (amino and nucleic acid sequences respectively) correspond to a CcaS mutant with a A92V point mutation that results in with improved photoswitching with PΦB.

SEQ ID NOs 3 and 4 (amino and nucleic acid sequences respectively) correspond to a CcaS mutant with a truncation (removal of bases 1-69) and the addition of an NLS sequence (as described in SEQ ID NO: 26 and 27).

SEQ ID NOs 5 and 6 (amino and nucleic acid sequences respectively) correspond to a CcaS mutant with a A92V point mutation that results in improved photoswitching with PPB and a truncation (removal of bases 4-69).

SEQ ID NOs 7 and 8 (amino and nucleic acid sequences respectively) correspond to a CcaS mutant with a A92V point mutation that results in improved photoswitching with PPB, the addition of an NLS sequences, and a truncation (removal of bases 1-69).

SEQ ID NOs 9 and 10 (amino and nucleic acid sequences respectively) correspond to a CcaS mutant with a A92V point mutation that results in improved photoswitching with PPB, the addition of an NLS sequences, a truncation (removal of bases 1-69), and the addition of a peptide tail (amino acids 1-20) encoding a 2A ribosomal skipping sequence.

SEQ ID NOs 11 and 12 (amino and nucleic acid sequences respectively) correspond to a CcaS mutant with a A92V point mutation that results improved photoswitching with PPB, the addition of an NLS sequences, a truncation (removal of bases 1-69), and the addition of a peptide tail (amino acids 1-29) encoding a 2A ribosomal skipping sequence.

### CcaR Variants

SEQ ID NOs 13 and 14 (amino and nucleic acid sequences respectively) correspond to a CcaR variant with an NLS and VP64 domain fused to the N-terminal as well as an N-terminal proline.

SEQ ID NOs 15 and 16 (amino and nucleic acid sequences respectively) correspond to a CcaR variant with an NLS and VP64 domain fused to the C-terminal as well as an N-terminal proline.

The term “variant” or “functional variant” as used throughout with reference to any of SEQ ID NOs: 1 to 50 refers to a variant gene sequence or part of the gene sequence which retains the biological function of the full non-variant sequence. A functional variant also comprises a variant of the gene of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence that results in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

As used in any aspect of the invention described throughout a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

In one embodiment, the “CcaS” protein encodes a light responsive histidine kinase, wherein the kinase is characterised by a number of domains or motifs. For example, the CcaS protein may comprise at least one of a GAF domain or GAF domain variant (for example, from AnPixjg2, slr1393g2, NpR1597g4 and UirSg), a His-Kinase domain and a nuclear localisation signal or NLS, as well as optionally at least one, preferably two PAS (or Per-Arnt-Sim) domains.

In one embodiment, the sequence of these domains comprises or consists of the following sequence or a functional variant thereof:

GAF domain (nucleic acid sequence):
(SEQ ID NO: 18):
ATCAGACAATCTCTTAATTTGGAGACTGTTTTGAACACTACAG
TTGCTGAAGTTAAGACACTTTTGCAGGTTGATAGAGTTCTTAT
CTATAGAATCTGGCAAGATGGTACAGGATCTGTTATCACTGAG
TCTGTTAATGCTAACTACCCTTCTATTTTGGGTAGAACTTTTT
CTGATGAGGTTTTCCCAGTTGAATATCATCAAGCTTACACAAA
GGGAAAAGTTAGAGCTATTAATGATATCGATCAGGATGATATC
GAAATCTGTCTTGCTGATTTCGTTAAACAATTCGGTGTTAAGT
CTAAACTTGTTGTTCCTATCTTGCAGCATAATAGAGCTTCTTC
TTTGGATAACGAATCTGAGTTTCCATATCTTTGGGGACTTTTG
ATTACACATCAGTGTGCTTTCACTAGACCTTGGCAACCTTGGG
AAGTTGAGCTTATGAAGCAGTTGGCTAACCAAGTTGCTATTGC
TATC
GAF domain (amino acid sequence):
(SEQ ID NO: 19):
IRQSLNLETVLNTTVAEVKTLLQVDRVLIYRIWQDGTGSVITE
SVNANYPSILGRTFSDEVFPVEYHQAYTKGKVRAINDIDQDDI
EICLADFVKQFGVKSKLVVPILQHNRASSLDNESEFPYLWGLL
ITHQCAFTRPWQPWEVELMKQLANQVAIAI
PAS domain (nucleic acid sequence);
domain 1: (SEQ ID NO: 20):
ACTAACCATACACTTCAGTCTTTGATTGCTGCTTCTCCTAGAG
GTATCTTTACTCTTAATTTGGCTGATCAAATTCAGATCTGGAA
CCCAACAGCTGAGCGAATCTTCGGATGGACTGAAACAGAGATT
ATCGCTCATCCTGAGCTTTTGACATCTAACATCCTTTTGGAAG
ATTACCAACAGTTTAAGCAAAAGGTTCTTTCTGGTATGGTTTC
TCCATCT
PAS domain (amino acid sequence); domain 1:
(SEQ ID NO: 21):
TNHTLQSLIAASPRGIFTLNLADQIQIWNPTAERIFGVVTETE
IIAHPELLTSNILLEDYQQFKQKVLSGMVSPS
PAS domain (nucleic acid sequence);
domain 2: (SEQ ID NO: 22):
ATCGATGATCCTGGACCAAGAATCCTTTATGTTAATGAGGCTT
TCACTAAGATCACAGGATACACTGCTGAAGAGATGTTGGGAAA
GACTCCTAGAGTTCTTCAAGGACCAAAAACTTCAAGAACTGAG
TTGGATAGAGTTAGACAGGCTATCTCTCAATGG
PAS domain (amino acid sequence); domain 2:
(SEQ ID NO: 23):
IDDPGPRILYVNEAFTKITGYTAEEMLGKTPRVLQGPKTSRTE
LDRVRQAISQW
His-Kinase domain (nucleic acid sequence):
(SEQ ID NO: 24)
ATGGCTTCTCATGAGTTTAGAACACCACTTTCTACTGCTTTGG
CTGCTGCTCAACTTCTTGAAAATTCTGAAGTTGCTTGGCTTGA
TCCTGATAAGAGATCAAGAAACCTTCATAGAATCCAAAATTCT
GTTAAAAACATGGTTCAACTTTTGGATGATATCTTGATTATCA
ACAGAGCTGAGGCTGGAAAGCTTGAGTTTAATCCAAACTGGCT
TGATTTGAAGCTTTTGTTCCAACAGTTCATTGAAGAGATCCAG
CTTTCTGTTTCTGATCAATACTACTTCGATTTCATCTGTTCTG
CTCAAGATACTAAGGCTCTTGTTGATGAAAGATTGGTTAGATC
TATCCTTTCTAATCTTTTGTCTAACGCTATCAAGTACTCTCCT
GGAGGTGGACAGATTAAAATCGCTCTTTCTTTGGATTCTGAGC
AGATTATCTTCGAAGTTACAGATCAAGGTATTGGAATCTCTCC
TGAGGATCAAAAGCAGATCTTTGAACCATTCCATAGAGGAAAG
AATGTTAGAAACATTACTGGTACAGGACTTGGTTTGATGGTTG
CTAAGAAATGTGTTGATCTTCATTCTGGATCTATCCTTTTGAA
GTCTGCTGTGGATCAAGGAACAACTGTGACCATCTGTCTCAAA
AGGTACAAC
His-Kinase domain (amino acid sequence):
(SEQ ID NO: 25)
MASHEFRTPLSTALAAAQLLENSEVAWLDPDKRSRNLHRIQNS
VKNMVQLLDDILIINRAEAGKLEFNPNWLDLKLLFQQFIEEIQ
LSVSDQYYFDFICSAQDTKALVDERLVRSILSNLLSNAIKYSP
GGGQIKIALSLDSEQIIFEVTDQGIGISPEDQKQIFEPFHRGK
NVRNITGTGLGLMVAKKCVDLHSGSILLKSAVDQGTTVTICLK
RYN
NLS (nucleic acid sequence):
(SEQ ID NO: 26)
TTACAACCAAAGAAGAAAAGGAAGGTGGGTGGA
NLS (amino acid sequence): (SEQ ID NO: 27)
LQPKKKRKVGG

Accordingly, in one embodiment, a CcaS variant may have at least one of a GAF domain, a NLS and a His-Kinase domain and optionally at least one, preferably at least two PAS domains as defined above or a domain with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% overall sequence identity to any one of SEQ ID NOs 18 to 27.

In one embodiment, the “CcaR” protein encodes a transcriptional regulatory protein, wherein the regulator is characterised by a number of domains or motifs. For example, the CcaR may comprise at least one of a REC domain (receiver domain, preferably a N-terminal REC domain), a transcriptional activation or repression domain and a DNA-binding domain (preferably a C-terminal DNA-binding domain). Preferably, CcaR comprises a VP64 transactivation domain.

In one embodiment, the sequence of these domains comprises or consists of the following sequences:

REC domain (nucleic acid sequence):
(SEQ ID NO: 28)
AGAATACTCCTCGTGGAAGATGATTTGCCATTAGCAGAAACCC
TCGCAGAAGCTTTGTCTGATCAACTTTACACTGTTGATATTGC
TACAGATGCTTCTTTGGCTTGGGATTATGCTTCTAGACTTGAA
TACGATTTGGTTATTCTTGATGTTATGTTGCCTGAGCTTGATG
GAATTACTCTTTGTCAGAAGTGGAGATCTCATTCTTATTTGAT
GCCAATCCTTATGATGACTGCTAGAGATACAATTAATGATAAG
ATCACAGGACTTGATGCTGGTGCTGATGATTACGTTGTTAAAC
CTGTTGATTTGGGTGAACTTTTTGCTAGAGTTAGAGCTCTTTT
G
REC domain (amino acid sequence):
(SEQ ID NO: 29)
RILLVEDDLPLAETLAEALSDQLYTVDIATDASLAWDYASRLE
YDLVILDVMLPELDGITLCQKWRSHSYLMPILMMTARDTINDK
ITGLDAGADDYVVKPVDLGELFARVRALL
DNA binding domain (nucleic acid sequence):
(SEQ ID NO: 30):
CAACCAGTTTTGGAGTGGGGTCCTATTAGACTTGATCCATCTA
CTTATGAAGTTTCTTACGATAATGAGGTTTTGTCTCTTACAAG
AAAGGAATACTCTATCTTGGAGCTTTTGCTTAGAAACGGAAGA
AGAGTTCTTTCTAGATCTATGATCATCGATTCTATCTGGAAGT
TGGAGTCTCCTCCAGAAGAGGATACAGTTAAAGTTCATGTTAG
ATCTTTGAGACAAAAGCTTAAGTCTGCTGGACTTTCTGCTGAT
GCTATTGAAACTGTTCATGGAATCGGTTACAGATTGGCTAAT
DNA binding domain (amino acid sequence):
(SEQ ID NO: 31):
QPVLEWGPIRLDPSTYEVSYDNEVLSLTRKEYSILELLLRNGR
RVLSRSMIIDSIWKLESPPEEDTVKVHVRSLRQKLKSAGLSAD
AIETVHGIGYRLAN
NLS (nucleic acid sequence):
(SEQ ID NO: 32)
CTCCAGCCTAAGAAGAAGAGAAAGGTTGGAGGT
NLS (amino acid sequence): (SEQ ID NO: 33)
LQPKKKRKVGG
VP64 domain (nucleic acid sequence):
(SEQ ID NO: 34):
GATGCCCTCGACGATTTCGACCTCGATATGCTCGGTTCTGATG
CTCTCGATGACTTTGACCTTGACATGCTTGGATCAGACGCTTT
GGACGACTTCGACTTGGACATGTTGGGATCTGATGCACTTGAT
GATTTTGACCTTGATATGCTT
VP64 domain (amino acid sequence):
(SEQ ID NO: 35):
DALDDFDLDMLGSDALDDFDLDMLGSDALDDFDLDMLGSDALD
DFDLDML

Accordingly, in one embodiment, a CcaR variant has at least one of a REC domain a NLS and a transcriptional activation or repression domain as defined in SEQ ID NO: 28 to 35 or a domain with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% overall sequence identity to SEQ ID NO 28 to 35.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

In a further embodiment, a variant as used herein, can comprise a nucleic acid encoding a LRHK or RR as defined herein that is capable of binding or hybridising under stringent conditions as defined herein to a nucleic acid sequence as defined in any of SEQ ID NOs 1 to 50.

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12 hours. Stringent conditions may also be achieved with the addition of destabilizing agents such as formamide.

In another embodiment, the construct further comprises at least one regulatory sequence operably linked to at least one of the light-responsive histidine kinase and the response regulator. In one embodiment, the construct comprises a first regulatory sequence operably linked to the LRHK. In a second embodiment, the construct comprises a second regulatory sequence operably linked a second regulatory sequence. However, preferably, the construct comprises a single regulatory sequence that is operably linked to both the LRHK and the RR.

To allow two proteins to be expressed as individual proteins from a single mRNA molecule, ribosomal skipping sequences may be added to the 5′ and/or 3′ end of the LRHK and/or RR gene. During translation, when the ribosome encounters a ribosomal skipping sequence it is prevented from creating the peptide bond with the last proline in the ribosomal skipping sequence. As a result, translation is stopped, the nascent polypeptide released and translation is re-initiated to produce a second polypeptide. This results in the addition of a C-terminal ribosomal skipping sequence (or the majority of such a sequence) to the first polypeptide chain, and a N-terminal proline to the next polypeptide.

Accordingly, in a further embodiment, the nucleic acid construct comprises at least one ribosomal skipping sequence.

In one example, the ribosomal skipping sequence may be selected from one of the following:

F2A; A 2A DNA sequence variant used
between two CDS.
F2A:
(SEQ ID NO: 36)
GGACAACTTCTCAACTTTGACTTGCTAAAGTTA
GCTGGTGATGTTGAATCTAATCCTGGACCA.

Use of the F2A sequence results in the addition of the F2Aaa1-20 polypeptide sequence to the C-terminus of the protein upstream of the ribosomal skipping site and a proline residue (F2Aaa21) to the downstream protein.

F2Aaa1-20:
(SEQ ID NO: 37)
GQLLNFDLLKLAGDVESNPG
F2Aaa21: P
F2A30; A 2A DNA sequence variant used
between two CDS.
F2A30:
(SEQ ID NO: 38)
CACAAACAGAAAATTGTGGCACCGGTGAAGCAGACTCTC
AACTTTGACTTGCTAAAGTTAGCTGGTGATGTTGAATCT
AATCCTGGACCA.

Use of the F2A30 sequence results in the addition of the F2A30aa1-29 polypeptide sequence to the C-terminus of the protein upstream of the ribosomal skipping site and a proline residue (F2A30aa30) to the downstream protein.

(SEQ ID NO: 39)
F2Aaa1-20: HKQKIVAPVKQTLNFDLLKLAGDVESNPG
F2Aaa21: P

In one embodiment, LRHK includes a C-terminal skipping sequence, preferably F2A30(aa1-29). The nucleic acid and amino acid sequence of CcaS with such a skipping sequence is shown in SEQ ID 9 and 11 and 10 and 12 respectively. Accordingly, where the nucleic acid construct comprises a single sequence for LRHK and RR, the LRHK preferably comprises a sequence comprising or consisting of SEQ ID NO: 10 or 12.

In a further embodiment, RR includes a N-terminal skipping sequence and F2A30(aa30), i.e. a proline amino acid residue. The nucleic acid and amino acid sequence of CcaR comprising such a skipping sequence is shown in SEQ ID 14 and 16 and 13 and 15 respectively. Accordingly, where the nucleic acid construct comprises a single sequence for LRHK and RR, RR preferably comprises a sequence comprising or consisting of SEQ ID NO: 14 or 16.

In a further alternative embodiment, an internal ribosomal entry site (IRES), tRNA sequence, a ribozyme (such as a Hammerhead (HH) ribozyme unit and/or a hepatitis delta virus (HDV) ribozyme unit) or direct repeat (DR) sequence could be used instead of a ribosomal skipping sequence. Again, such sequences may be added to the 5′ and/or 3′ end of the LRHK and/or RR gene and allow two proteins to be expressed as individual proteins from a single mRNA transcript and from a single regulatory sequence (promoter).

In a further embodiment, the nucleic acid construct may further comprise a reporter sequence. The reporter sequence may be used as a means to flag cells that have been successfully transformed with the nucleic acid construct. The reporter sequence may also be used as a control to allow quantification of the level of expression of a target gene, expressed concurrently (either on the same or on a different expression vector) as the vector comprising the LRHK and/or the RR. Accordingly, the reporter sequence may be any sequence that can perform this function. As an example, common tags include the fluorescent proteins, such as GFP, EGFP, Emerald, Superfolder GFP, Azami Green, mWasabi, TagGFP, TurboGFP, AcGFP, ZsGreen, T-Sapphire, EBFP, EBFP2, Azurite, mTagBFP, ECFP, mECFP, Cerulean, mTurquoise, CyPet, AmCyan 1, Midori-Ishi Cyan, TagCFP, mTFP1, EYFP, Topaz, Venus, mCitrine, YPet, TagYFP, PhiYFP, ZsYellowl, mBanana, Kusabira Orange Kusabira Orange2 mOrange mOrange2 dTomato dTomato-Tandem, TagRFP, TagRFP-T, DsRed, DsRed2, DsRed-Express (T1), DsRed-Monomer, mTangerine, mRuby, mApple, mStrawberry, AsRed2, mRFP1, JRed, mCherry, HcRed1, mRaspberry, dKeima-Tandem, HcRed-Tandem, mPlum and AQ143.

In a further embodiment, the regulatory sequence is operably linked to a regulatory sequence. Preferably the regulatory sequence is operably linked to a single regulatory sequence that is also operably linked to the LRHK and/or the RR. As discussed above, the reporter sequence may also comprise 5′ or 3′ ribosomal skipping sequences, such as one of the skipping sequences described above.

The term “operably linked” as used throughout refers to a functional linkage between the promoter sequence and the gene of interest, such that the promoter sequence is able to initiate transcription of the gene of interest.

In a further embodiment, the construct comprises at least one terminator sequence, which marks the end of the operon causing transcription to stop. A suitable terminator sequence would be well known to the skilled person, and may include Rho-dependent and Rho-independent sequences. In one example, the sequence may comprise or consist of SEQ ID NO: 42 and/or 43 or a functional variant thereof.

In one embodiment, the regulatory sequence is a promoter. According to all aspects of the invention, including the method above and including the plants, methods and uses as described below, the term “regulatory sequence” is used interchangeably herein with “promoter” and all terms are to be taken in a broad context to refer to regulatory nucleic acid sequences capable of effecting expression of the sequences to which they are ligated. The term “regulatory sequence” also encompasses a synthetic fusion molecule or derivative that confers, activates or enhances expression of a nucleic acid molecule in a cell, tissue or organ.

The term “promoter” typically refers to a nucleic acid control sequence located upstream from the transcriptional start of a gene and which is involved in the binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences.

In a preferred embodiment, the promoter is a constitutive promoter, strong promoter or tissue-specific promoter.

A “constitutive promoter” refers to a promoter that is transcriptionally active during most, but not necessarily all, phases of growth and development and under most environmental conditions, in at least one cell, tissue or organ. Examples of constitutive promoters include the cauliflower mosaic virus promoter (CaMV35S or 19S), rice actin promoter, maize ubiquitin promoter, polyubiquitin (UBQ10) promoter, rubisco small subunit, maize or alfalfa H3 histone, OCS, SAD1 or 2, GOS2 or any promoter that gives enhanced expression.

A “strong promoter” refers to a promoter that leads to increased or overexpression of the target gene. Examples of strong promoters include, but are not limited to, CaMV-35S, CaMV-35Somega, Arabidopsis ubiquitin UBQ1, rice ubiquitin, actin, Maize alcohol dehydrogenase 1 promoter (Adh-1), AtPyk10, BdEF1α, FaRB7, HvIDS2, HvPht1.1, LjCCaMK, MtCCaMK, MtIPD3, MtPT1, MtPT2, OsAPX, OsCc1, OsCCaMK, OsCYCLOPS, OsPGD1, OsR1G1B, OsRCc3, OsRS1, OsRS2, OsSCP1, OsUBI3, SbCCaMK, SiCCaMK, TobRB7, ZmCCaMK, ZmEF1α, ZmPIP2.1, ZmRsyn7, ZmTUB1α, ZmTUB2α and ZmUBI.

Tissue specific promoters are transcriptional control elements that are only active in particular cells or tissues at specific times during plant development.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes are known to the skilled person and include for example beta-glucuronidase or beta-galactosidase.

In one embodiment, the nucleic acid construct further comprises a target sequence operably linked to a regulatory sequence that is specifically activated by the response regulator. In an alternative embodiment, the regulatory sequence is constitutively active and binding of RR represses the activity of the regulatory sequence. Preferably the regulatory sequence is a promoter, more preferably an inducible promoter. In a preferred embodiment, the promoter comprises a core promoter element (such that the promoter has little or no activity without adjacent or distal activation sequences) and a cis-regulatory element (CRE) (non-variant or variant) recognised by CcaR. In one example, the core promoter element may comprise or consist of a sequence as defined in SEQ ID NO: 41 or a variant thereof and the CRE may comprise or consist of a sequence as defined in SEQ ID NO: 40 or a variant thereof. In a further preferred embodiment, the promoter comprises or consists of the nucleic acid sequence as defined in SEQ ID NO: 17 or a functional variant thereof. In one embodiment, the target sequence may be expressed using a promoter that drives overexpression. Overexpression according to the invention means that the target gene is expressed at a level that is higher than the expression of the endogenous target gene whose expression is driven by its endogenous counterpart.

As used herein a “target sequence” may refer to any nucleic acid sequence or gene that could possibly be and/or would be of value to control the transcription level of.

The construct may further comprise a second terminator sequence to define the end of the target sequence operon. A terminator sequence is defined above. Preferably the terminator sequence comprises or consists of SEQ ID NO: 43 or a variant thereof.

As described in detail below, in use when the (LRHK) is exposed to an activating wavelength of light it phosphorylates the RR, which then binds to its cognate promoter (the regulatory sequence that is specifically recognized by the RR) resulting in transcription of the target sequence.

In another aspect of the invention, there is provided a vector or expression vector comprising the nucleic acid construct described herein. In one embodiment, the vector backbone is pEAQ.

In another aspect of the invention there is provided a host cell comprising the nucleic acid construct or the vector. The host cell may be a prokaryotic or eukaryotic cell. Preferably the cell is a mammalian, bacterial or plant cell. Most preferably the cell is a plant cell.

In another aspect of the invention there is provided a transgenic organism where the transgenic organism expresses the nucleic acid construct or vector. Again, the organism is any prokaryote or eukaryote, but in a preferred embodiment, the organism is a plant.

In one embodiment, the progeny organism is transiently transformed with the nucleic acid construct or vector. In another embodiment, the progeny organism is stably transformed with the nucleic acid construct described herein and comprises the exogenous polynucleotide which is heritably maintained in at least one cell of the organism. The method may include steps to verify that the construct is stably integrated. Where the organism is a plant, the method may also comprise the additional step of collecting seeds from the selected progeny plant.

In a further aspect of the invention there is provided a method of producing a transgenic organism as described herein. In a different aspect there is provided a method of producing an organism that is capable of light-regulated expression of a target sequence. In either aspect the method comprises at least the following steps:
a. selecting a part of the organism;
b. transfecting at least one cell of the part of the organism of part (a) with the nucleic acid construct or the vector; and
c. regenerating at least one organism derived from the transfected cell or cells.

Transformation or transfection methods for generating a transgenic organism of the invention are known in the art. Thus, according to the various aspects of the invention, a nucleic acid construct as defined herein is introduced into an organism and expressed as a transgene. The nucleic acid construct is introduced into said organism through a process called transformation. The term “transfection”, “introduction” or “transformation” as referred to herein encompasses the transfer of an exogenous polynucleotide into a host cell, irrespective of the method used for transfer. Such terms can also be used interchangeably in the present context. Where the organism is a plant, tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

Transformation of plants is now a routine technique in many species. Advantageously, any of several transformation methods may be used to introduce the gene of interest into a suitable ancestor cell. The methods described for the transformation of an organism's cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker or expression of a constitutively expressed reporter gene, as described above. Following DNA transfer and regeneration, putatively transformed plants may also be evaluated, for instance using Southern blot analysis, for the presence of the gene of interest, copy number and/or genomic organisation. Alternatively or additionally, expression levels of the newly introduced DNA may be monitored using Northern and/or Western blot analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further aspect of the invention, there is provided a plant obtained or obtainable by the methods described herein.

In another aspect of the invention there is provided a method of modulating expression of a target gene in an organism, the method comprising introducing and expressing at least one nucleic acid construct or vector as described herein in an organism, and applying at least one (activating and/or repressing) wavelength of light, wherein preferably the wavelength of light modulates expression of the target gene, as described herein. In one embodiment, the wavelength of light activates or represses activation of a LRHK. As described above, preferably the wavelength of light activates the LRHK causing phosphorylation of RR which then binds to its cognate promoter to drive transcription of the target gene. As such, as used throughout an “activating” wavelength is one that activates LRHK, and preferably causes the expression or increases the expression of target gene (although in alternative embodiments an activating wavelength may decrease expression of a target gene). Similarly, as also used throughout, a “repressing” wavelength of light is one that represses or prevents activation of LRHK, and preferably decreases or prevents the expression of a target gene, although, again in alternative embodiments, the repressing wavelength may increase expression of a target gene.

Preferably the target gene is operably linked to a regulatory sequence that may be specifically activated by the response regulator, as described above. Even more preferably, the target gene is a transgene (either an exogenous or endogenous transgene) operably linked the regulatory sequence.

In one embodiment, the nucleic acid construct comprises a LRHK and a RR operably linked to at least one regulatory sequence, as described herein. Preferably, the construct also comprises a target gene operably linked to a regulatory sequence that may be specifically activated by the response regulator, as also described above.

In a further embodiment, the method may comprise introducing and expressing a first and second nucleic acid construct, wherein the first nucleic acid construct comprises a LRHK operably linked to a regulatory sequence and the second nucleic acid construct comprises a RR operably linked to a regulatory sequence. In a further preferred embodiment, the method may further comprise introducing a third nucleic acid construct, wherein the third nucleic acid construct comprises a target gene operably linked to a regulatory sequence that may be specifically activated by the response regulator. Alternatively, the target gene and regulatory sequence may be present on the first or second nucleic acid construct.

As used herein “modulating” may encompass an increase or decrease in expression of a target gene, preferably compared to the level of expression in a control organism. In particular, expression of a target gene may be increased by applying a wavelength of light, preferably a first activating or repressing wavelength of light. Expression of the target gene can then be decreased (or further increased) by applying a second wavelength of light that is different from the first wavelength of light and is applied after the first wavelength of light. This effect can again be reversed by subsequently applying an activating wavelength of light and so on. The result is an “on/off” system to control expression of a target gene. However, the present invention is also capable of more subtlety than a simple “on/off” switch for target gene expression. We have found that different wavelengths of light can stimulate or repress target gene expression to different levels.

Accordingly, in a further embodiment, the activating light wavelength can be a maximal activating wavelength or an intermediate activating wavelength. In such an example, the maximal activating wavelength results in the highest level of target gene expression—i.e. a level of target gene expression that is higher than the intermediate activating wavelength. Similarly, the intermediate activating wavelength results in expression of the target gene but to a level that is lower than that obtained by applying a maximal activating wavelength. By comparison, the repressing wavelength of light results in no or minimal expression of the target gene.

In one embodiment, the level of target gene expression may be relative to a control organism, such as a plant, wherein the control plant does not express the transgene—for example, the plant does not express a nucleic acid construct, as described herein.

In an alternative embodiment, that may be particularly useful for defining a maximal or intermediate wavelength of light, the level of target gene expression may be relative to the level of gene expression in an organism where the light applied is white light or dark light (as defined below).

In a preferred embodiment of the methods described herein the organism is grown or cultured in light and/or darkness (darkness as used in this context refers to growth in the absence of light). In other words, the organism may be cultured in normal day and/or night conditions (normal day and/or night conditions for that organism or any experimentally set conditions). Where the organism is a plant, this may mean that the plant is exposed to a suitable day/night cycle. As such, expression of a target gene can be modulated (i.e. increased or decreased as defined herein) by the application of a (activating or repressing) wavelength of light in additional to normal light/dark conditions—this may lead to enriched white light for example (e.g. white light enriched with red or blue light). Accordingly, in a further embodiment, the increase or decrease in the level of target gene expression following application of an activating or repressing wavelength may be relative to the level of gene expression when the organism is cultured or grown in light or darkness (without application of a activating or repressing wavelength).

Accordingly, in a preferred embodiment, the method comprises applying enriched light, preferably enriched white light. In other words, the method comprises growing or culturing the organism in enriched light, preferably enriched white light.

As used here “white light” may refer to all visible light (for example, light between the wavelengths of 390 nm to 700 nm) or a combination of red, blue and green light as described below.

As used here “dark light” may refer to non-visible light. For example, dark light may refer to light in the infra-red portion (and beyond) of the spectrum (for example, above 700 nm, more preferably above 750 nm, and even more preferably between 710 and 850 nm) or light in the ultra-violet portion (and beyond) of the spectrum (for example, 390 nm, more preferably between 10 and 400 nm).

As used here, “enriched light”, preferably enriched white light may comprise a proportion of activating or repressing wavelength of light, wherein said activating or repressing wavelength of light may be as defined below, and wherein the proportion of the activating or repressing wavelength of light is at least 5%, 10%, 20%, 25%, 30%, 35%, 40%, 45%, 50%, 55%, 60%, 65%, 70%, 75%, 80%, 85%, 90% or 95% of the total light.

Accordingly, modulating target gene expression encompasses both turning on, and optionally turning off expression of a target gene, as well as modulating the level of increase or decrease of target gene expression. As explained above, this latter feature allows the system to exhibit a first level of target gene expression during normal-light dark cycles and a second, different level of target gene expression (that is either higher or lower than the first) following application of a specific light spectra (such as red, blue or green) that is not found in a normal horticultural environment. As such, the invention allows for the very precise control of levels of target gene expression. Moreover, as the invention depends on the application of light to modulate gene expression, expression of a target gene can also be controlled (i.e. modulated) spatially (e.g. by directing the light source at a specific location on the organism) and temporally (e.g. by applying an activating or repressing wavelength at any point during the growth or life cycle of an organism).

As used throughout “increase”, “higher” or “activate” (such terms may be used interchangeably) may mean an increase in target gene expression of at least 5%, 10%, 20%, 25%, 30%, 35%, 40%, 45%, 50%, 55%, 60%, 65%, 70%, 75%, 80%, 85%, 90% or 95% or more compared to a control as described above. Similarly, as also used throughout, “further increasing” the expression of a target gene in response to the application of a second wavelength of light may mean an increase in target gene expression of at least 5%, 10%, 20%, 25%, 30%, 35%, 40%, 45%, 50%, 55%, 60%, 65%, 70%, 75%, 80%, 85%, 90% or 95% or more compared to the level of gene expression following application of the first wavelength of light.

As also used throughout, “decrease” or “repress” (such terms may also be used interchangeably) may mean an decrease in target gene expression of at least 5%, 10%, 20%, 25%, 30%, 35%, 40%, 45%, 50%, 55%, 60%, 65%, 70%, 75%, 80%, 85%, 90% or 95% or more compared to a control as described above. Alternatively, such a decrease may be relative to the level of gene expression following application of the first wavelength of light.

In one embodiment, the activating wavelength of light may fall within one of the following ranges 430-495 nm (blue light), 495 to 570 nm (green light), 600 to 750 nm (red light). Alternatively the wavelength may be described as dark light (as described above) or white light (as described above). In another embodiment, the activating wavelength of light may comprise white light, as described above, supplemented or enriched with a specific wavelength of light, for example, blue, green or red light. This latter option may be particularly valuable where the organism is a plant, and wherein the plant requires white light for growth, but can tolerate an additional specific light wavelength, such as blue or red light with minimal physiological effects.

In a further embodiment, the maximally activating wavelength of light preferably falls within one of the following ranges range of 600 to 750 nm (red light). In an alternative embodiment, the intermediate activating wavelength preferably falls within the range 390 nm to 700 nm (white light) or 495 to 570 nm (green light).

In an alternative embodiment, the repressing wavelength of light may fall within one of the following ranges, 430-495 nm (blue light), 495 to 570 nm (green light) and 600 to 750 nm (red light). Alternatively the light may be white light, as defined above or dark light. In a preferred embodiment, the repressing wavelength of light falls within the range 430-495 nm (blue light). In another embodiment, the repressing wavelength of light may comprise white light, as described above, supplemented or enriched with a specific wavelength of light, for example, blue, green or red light.

In one embodiment, the activating or repressing wavelength of light is applied for sufficient time to modulate target gene expression as described above. Depending on the system and organism, the length of time could be seconds, minutes, hours or days. In one example, the light may be applied for at least 6 hours, more preferably at least 12 hours and even more preferably at least 18 hours.

It would be clear to the skilled person that other wavelengths of light, both in the visible and non-visible spectrum, and/or falling within the ranges described above, would be possible. The above ranges are intended as examples only.

In one embodiment, the light is applied using a light source having a desired wavelength as described above. Suitable light sources would be known to the skilled person, but may be one or more of a suitable LED, laser, white light source and the like.

In one example, the organism is cultured or grown for at least 1 hour, preferably at least 2, 6, 12 or 24 hours, or 2, or 7 days before an activating and/or repressing wavelength of light is applied.

In one embodiment, the activating and/or repressing wavelength of light is preferably applied to an outer or external surface of the organism. Where the organism is a plant, this surface is preferably at least one leaf and/or at least one root and/or at least one shoot or stem.

In a further aspect of the invention, there is provided a method of modulating any biochemical pathway or response or biological process in a target organism, the method comprising introducing and expressing at least one nucleic acid construct or vector as described herein, and applying a (activating or repressing) wavelength of light, as described above. In one embodiment, the biochemical pathway is a developmental pathway or physiological response. Where the organism is a plant, the method may be used, for example, to modulate the concentration of phytohormones to modulate developmental traits such as organ size and plant architecture, to modulate flowering (i.e. prevent or induce flowering, including for purposes of synchronization), modulate germination (for example, prevent or induce germination, including for purposes of synchronization), modulate senescence (for example to prevent senescence in food products for increased shelf-life), modulate a stress response (for example, induce a drought stress response or produce drought stress tolerance) or modulate plant immunity (e.g. increase or decrease immunity to a plant pathogen or parasite). Alternatively, the method may be used to control expression or production of a natural or synthetic metabolite such as a pharmaceutical.

In a further aspect of the invention, there is provided the use of the nucleic acid or vector as described herein to modulate expression of a target gene.

In another aspect of the invention, there is provided a photoreceptor molecule, wherein the photoreceptor comprises a phytochrome or phytochrome-related photoreceptor protein and a chromophore. In one embodiment, the phytochrome-related photoreceptor is CcaS, as described herein. In one example, the chromophore is a tetrapyrrole. In one embodiment, the tetrapyrrole is selected from PCB (phycocyanobilin), PφB (phytochromobilin), phycoviolobilin or phycoerythrin and BV (biliverdin). Similarly, there is also provided the use of a photoreceptor molecule as described herein to modulate any biochemical pathway or response or biological process in a target organism.

In a further embodiment, the nucleic acid constructs described above may further comprise at least one biosynthetic enzyme necessary to produce a chromophore, as described above, preferably from heme. In one example, the biosynthetic enzyme may be heme oxygenase and/or oxidoreductase, such as heme oxygenase 1 (ho1) and phycocyanobilin:ferredoxin (pcyA).

In a further aspect of the invention, there is provided a nucleic acid construct comprising a target sequence operably linked to a regulatory sequence, wherein the regulatory sequence is specifically activated by the response regulator. In one embodiment, the regulatory sequence comprises or consists of a nucleic acid sequence as defined in SEQ ID NO: 17 or a functional variant thereof. A functional variant is defined above.

In a final aspect of the invention, there is provided a nucleic acid molecule comprising

a. a nucleic acid sequence encoding a polypeptide as defined in any of SEQ ID NOs 1, 3, 5, 7, 9, 11, 13 and 15;

b. a nucleic acid sequence as defined in any of SEQ ID NOs 2, 4, 6, 8, 10, 12, 14, 16, 17, 47, 48, 49 or 50 or the complementary sequence thereof;

c. a nucleic acid with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the nucleic acid sequence of (a); or (b)

d. a nucleic acid sequence that is capable of hybridising under stringent conditions as defined herein to the nucleic acid sequence of any of (a) to (c).

The term “organism” as used herein refers to any prokaryotic or eukaryotic organism. Some examples of eukaryotes include a human, a non-human primate/mammal, a livestock animal (e.g. cattle, horse, pig, sheep, goat, chicken, camel, donkey, cat, and dog), a mammalian model organism (mouse, rat, hamster, guinea pig, rabbit or other rodents), an amphibian (e.g., Xenopus), fish, insect (e.g. Drosophila), a nematode (e.g., C. elegans), a plant, an algae, a fungus. Examples of prokaryotes include bacteria (e.g. cyanobacteria) and archaea.

The term “plant” as used herein may refer to any plant. For example, the plant may be a monocot or dicot. Preferably, the plant is a crop plant. By crop plant is meant any plant which is grown on a commercial scale for human or animal consumption or use. In a preferred embodiment, the plant is a cereal. In another embodiment the plant is Arabidopsis or Medicago truncatula. In another example, the plant may be N. benthamiana.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein. The term “plant” also encompasses plant cells, suspension cultures, callus tissue, embryos, meristematic regions, gametophytes, sporophytes, pollen and microspores, again wherein each of the aforementioned comprises the nucleic acid construct.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. Another product that may be derived from the harvestable parts of the plant of the invention is biodiesel. The invention also relates to food products and food supplements comprising the plant of the invention or parts thereof. In one embodiment, the food products may be animal feed. In another aspect of the invention, there is provided a product derived from a plant as described herein or from a part thereof.

In a most preferred embodiment, the plant part or harvestable product is a seed or grain. Therefore, in a further aspect of the invention, there is provided a seed produced from a transgenic or genetically altered plant as described herein.

In an alternative embodiment, the plant part is pollen, a propagule or progeny of the genetically altered plant described herein. Accordingly, in a further aspect of the invention there is provided pollen, a propagule or progeny produced from a transgenic or genetically altered plant as described herein.

A control organism, such as a plant as used herein according to all of the aspects of the invention is an organism that has not been modified according to the methods of the invention.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting example.

### Example 1

### The CcaS-CcaR System

The CcaS-CcaR system is a green/red photoswitchable two-component system derived from Synechocystis PCC6803 and consists of a light-responsive histidine kinase (LRHK), CcaS, and its cognate response regulator (RR), CcaR. CcaS is a membrane-associated cyanobacteriochrome which covalently binds a linear tetrapyrrole molecule, phycocyanobilin (PCB), to a conserved cysteine residue in its GAF domain. This allows for reversible photoactivation of CcaS with maximal activation in response to green light (˜535 nm) and maximal repression by red light (˜672 nm). Activating light wavelengths trigger CcaS to phosphorylate and activate CcaR, which then binds a cognate DNA recognition element, the cis-regulatory element (CRE), and promotes transcription of target gene(s) in cis.

Characterization of the Chromophore Dependency of the CcaS-CcaR System by Heterologous Expression in E. coli

In plants, the native chromophore for CcaS, PCB, is not produced, but the near identical chromophore, phytochromobilin (PΦB) is. We therefore set out to test if the CcaS-CcaR system would photoswitch in E. coli with PΦB.

The CcaS-CcaR system, in E. coli, is designed as a two-vector system. From one vector, CcaS is synthesized along with the two proteins, HO1 and PCYA, which produce the chromophore PCB from heme. From the second vector, CcaR is produced. The second vector also holds a sfgfp gene under the control of the P_{cpcg2-172} promoter. To produce PΦB, instead PCB, we replaced the pcyA gene with the gene encoding the PΦB synthase from Arabidopsis, lacking a transit peptide (mHY2), as described by Mukougawa et al. (2006)⁴. We also characterize the photoswitching of the system in the presence of the precursor molecule for PCB and PΦB, biliverdin (BV), and in the absence of any chromophore (Ø). In order to test this, we introduced stop mutations in pcyA and ho1, respectively.

Photoswitching Assay in E. coli. In order to examine the behaviour of the CcaS-CcaR system and its variants in E. coli, cells expressing the systems are cultured in defined light regimes and then tested for GFP fluorescence in a fluorimeter. GFP fluorescence serves as a reporter of photoactivation of CcaS and successful signal transduction through CcaR. An example of data from such an experiment is seen below (Error! Reference source not found.).

With PCB the CcaS-CcaR system is activated by a red-green-blue light mixture simulating white light (RGB-white), blue light, and green light and shows low activity in red light and in darkness (Error! Reference source not found.). With PΦB, the system appears to be constitutively active under all tested light conditions. Only subtle changes in activity are observed in response to the different light regimes. With BV, the system is inactivated by RGB-white, and blue light treatment. Low activity is observed under green and red light conditions and in darkness. Without the chromophore, the system is inactivated by RGB-white, and blue light treatment and the system only show very low activity under green and red light conditions and in darkness (Error! Reference source not found. 3).

Repurposing the CcaS-CcaR System for in Planta Function by Engineering in E. coli
a. We made several modifications to the CcaS-CcaR system for the purpose of creating a system that would function in plants. We tested some of these modifications in E. coli to confirm that the photoswitching function was not compromised. We also tested certain modifications in planta (described below).
b. Modifications to CcaS
   a. Improved the photoswitching of CcaS with PΦB
   b. Released CcaS from the cell membrane by removing its membrane anchor via a N-terminal deletion of 66 bases.
   c. N-terminal nuclear localization signal (NLS) added to CcaS
   d. Confirmed that peptide tails added by ribosomal skipping sequences were tolerated by CcaSs
      Improving Photoswitching of CcaS with PΦB

We first set out to adapt CcaS for improved photoswitching with PΦB by site-directed mutagenesis of residues in the chromophore binding pocket. By comparing sequences for proteins that utilize either phycoviolobilin (PVB), PCB, PΦB or BV as chromophores, including four cyanobacteriochromes (TePixJ, FdRcaE, SyCcaS and SyCph1), two bacteriophytochromes (PsBphP and DrBphP) and two plant phytochromes (AtPhyA and AtPhyB), we identified candidate amino acid residues that could be mutated in order to improve CcaS photoswitching with PΦB. The following 8 single amino acid residue mutations were created by site-directed mutagenesis of CcaS; L80M, I84F, A92V, I104Y, V113D, F114I, L142H and F149M. The A92V mutation improved CcaS photoswitching with POB but also altered the photochemical properties of the protein with respect to blue light and red light (Error! Reference source not found. 4). CcaS with the A92V mutation is from heron referred to as CcaS (A92V). Rather than being activated by blue light and RGB-white light and repressed by red light, the CcaS(A92V) with PΦB system is repressed by blue light and RGB-white light and activated by red light. The low activity in RGB-white light might be a result of the blue light response being dominant.

### Removing the Transmembrane Domain of CcaS to Make it Soluble and Adding a N-Terminal Nuclear Localization Signal

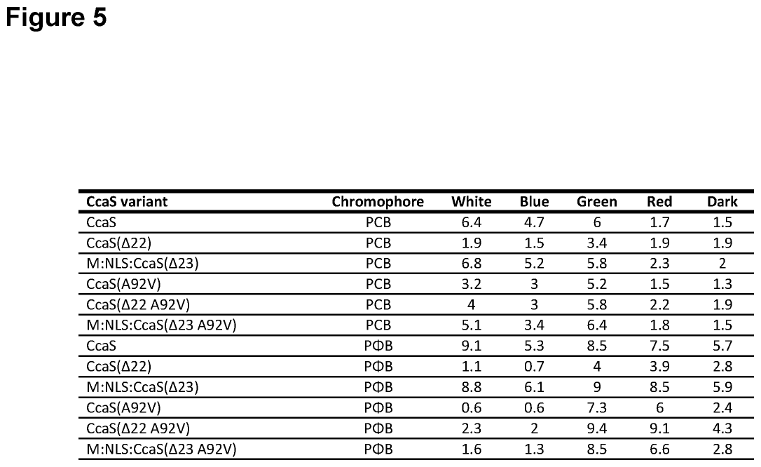

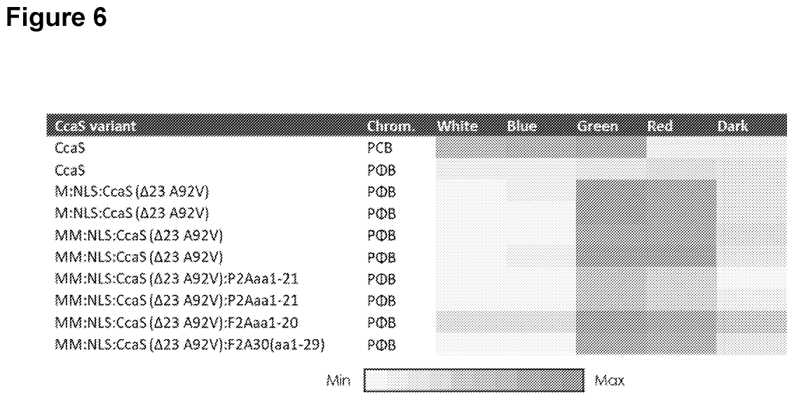

In order to release CcaS(A92V) from the cell membrane, bioinformatics software (Phobius and TMHMM-2.0) was used to predict the transmembrane domain (TMD). Phobius predicted the TMD to be encoded by bases 16-69 or 16-87 and TMHMM-2.0 predicted 13-69. A truncation was made, removing bases 4-69 in ccaS (corresponds to a G2_H23del in CcaS, referred to as Δ22). Δ22 was not well tolerated by CcaS. However, when removing bases 1-69 in ccaS (Corresponds to an M1_H23del in CcaS, referred to as Δ23) and replacing them with an NLS sequence, the photoswitching properties were restored (FIG. 5).

### Testing the Effects of 2A Peptide Tails on CcaS Functionality

Ribosomal skipping is a technology used to express multiple proteins from a single mRNA in eukaryotes and can therefore be used to minimize the size of an expression vector, because fewer promoter and terminator sequences are required. We wished to explore if this technology was compatible with our system. During translation, a 2A sequence will cause translation to stop, release the nascent peptide chain and reinitiate translation to produce a second peptide chain. During this process, a peptide tail encoding the majority of the 2A ribosomal skipping sequence, is added to the C-terminus of the upstream protein while a single proline is added to the N-terminus of the downstream protein. In order to test whether the addition of 2A peptide tails could affect CcaS function, we tested CcaS with three peptide tails, corresponding to the 2A sequences P2A, F2A and F2A₃₀, in E. coli photoswitching assays (Table 4). As 2A sequences are not functional in E. coli, the sequences encoding the 2A tails were added to the 3′ end of tested CcaS variant (MM:NLS:CcaS (Δ23 A92V)). The F2A tail was not well tolerated, but both the P2A and the F2A30 sequence were tolerated well (Error! Reference source not found. 6).

### Repurposing the CcaS-CcasaR System for in Planta Function by Engineering in Tobacco

For the system to function in planta, we had to make a plant expression vector and several further modifications to the system.
Further modifications to CcaS
   ccaS was codon optimized for expression in Arabidopsis.
Further modifications to CcaR
   C-terminal NLS signal added to CcaR
   VP64 eukaryotic transactivation domain added to CcaR
   ccaR was codon optimized for expression in Arabidopsis.
Constructed a synthetic cognate promoter for CcaR or ‘upstream activation sequence’ (UAS) consisting of three copies of a CcaR recognition element fused to a minimal CaMV 35S promoter sequence.
Add a GFP variant (NLS:Venus) as a fluorescence output reporter for light induced gene expression for the new system.
Add a GFP homolog (NLS:TagRFP) as a normalization control for expression of the system in plants.
F2A₃₀: Add ribosomal skipping sequences (e.g. F2A₃₀) between ccaS and tagrfp and between tagrfp and ccaR in order to express all three system components from the same promoter-terminator cassette.

### Design of the Plant Expression Vector

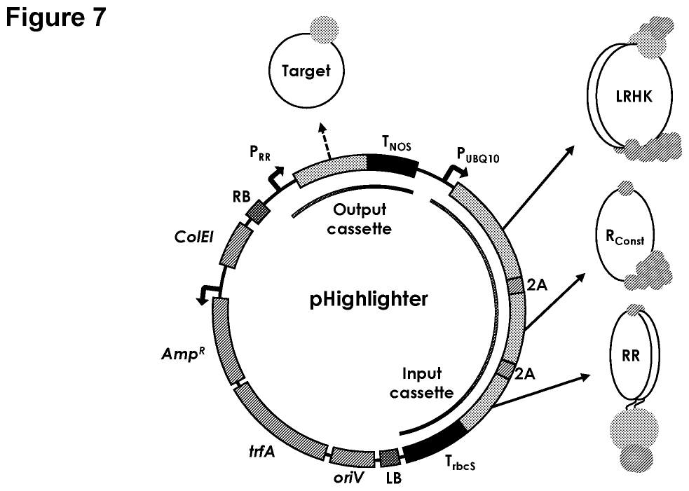

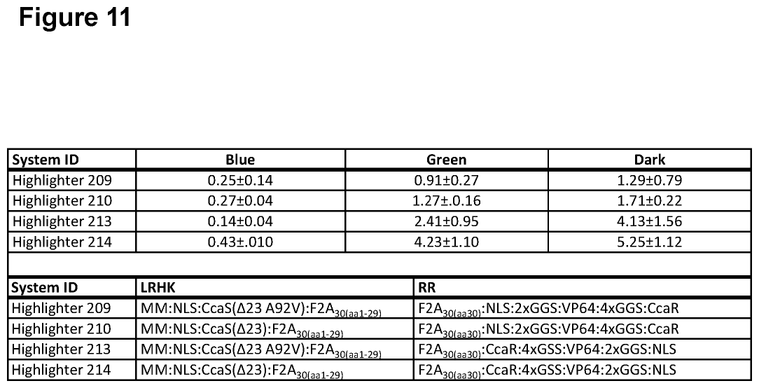

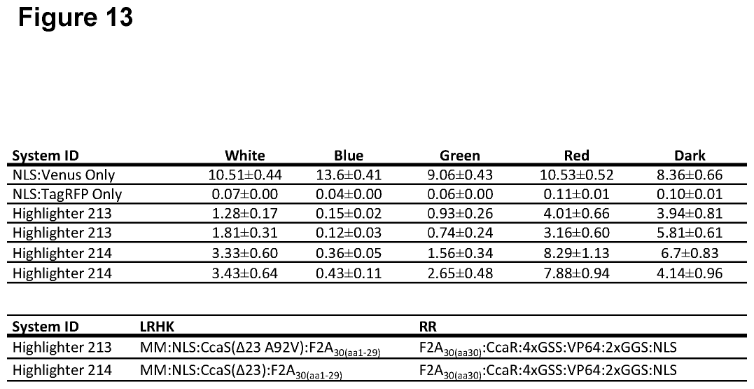

To express and test variants of the Highlighter system in planta we designed plant expression vectors with an input cassette and an output cassette. In principle, the input cassette expresses the proteins required for the Highlighter system to control expression of a target gene (Target) in planta via the output cassette. The input cassette was designed for constitutive expression of three proteins: a light-responsive histidine kinase (a CcaS variant), a reporter gene (TagRFP) and a repose regulator (a CcaR variant). The output cassette was designed with a synthetic cognate promoter (P_{RR}) that the response regulator can bind to and induce target gene expression in planta (FIG. 7).

### The Vector Backbone Used to Create Our Plant Expression Vector

The vector backbone used to build our plant expression vector, was obtained from collaborators at the DynaMo Center (University of Copenhagen, Associate Professor Meike Burow). The vector is based on pEAQ-HT but the region between the RB and LB has been replaced with a cassette containing P_{UBQ10}, a USER cassette and T_{rbcS}.

### Designing the Output Cassette: A Light-Controlled Gene Expression Cassette

The output cassette for the Highlighter system was designed as a gateway cassette (to allow for easy exchange of the expressed gene), with the sequence of the cognate promoter for the RR upstream of the cassette and a T_{NOS} sequence downstream. For our initial test, we decided to use NLS:Venus (NLS:edAFPt9) as the reporter to evaluate the light-induced gene expression.

### Designing a Synthetic Plant Promoter and Cognate Transcription Activator

A synthetic plant promoter and transcription activator was designed for the Highlighter system, based on the idea behind the estrogen inducible XVE system⁵. The XVE system is composed of a chimeric transcription activator, XVE (a fusion of the DNA-binding domain of the bacterial repressor LexA (X), the acidic transactivating domain of VP16 (V) and the regulatory region of the human estrogen receptor (E)), and its cognate promoter, which consists of eight copies of the LexA operator fused upstream of the −46 35S minimal promoter. In the presence of estrogen, XVE binds its cognate promoter and the downstream gene is transcribed.

Our synthetic promoter design consists of three copies of the ccaR CRE fused upstream of the −51 35S minimal promoter (FIG. 8). Inspired by the work of Qilai Huang et al.⁶, we mimicked their construct 191 so that the ccaR CREs were spaced evenly around the DNA helix, offset at 120° angles. This design was chosen as it effectively recruited transcription machinery components to the TATA box in eukaryotic HEK293T cells to form the transcription initiation complex.

### Designing the Input Cassette: An Expression Cassette for the LRHK and the RR

To keep the size of the expression vector to a minimum and to attempt to balance expression of the LRHK and RR, both LRHK and RR variants, along with an expression reporter (TagRFP), were expressed from a single cassette controlled by P_{UBQ10} and T_{rbcS}. To allow the three proteins to be expressed as individual proteins from one mRNA, F2A₃₀ ribosomal skipping sequence were included between ccaS and tagrfp and between tagrfp and ccaR. Because TagRFP will be constitutively expressed from the input cassette, we can quantify the induction of a fluorescent Target (e.g. NLS:Venus) ratiometrically by dividing the YFP signal by the RFP signal. The TagRFP also serves as a reporter for cells expressing the Highlighter system.

### Testing the Efficiency of Ribosomal Skipping of 2A Sequences in Planta (Transient Expression in Tobacco)

We tested the efficiency of ribosomal skipping of ‘2A-type’ sequences in planta by transient expression in N. benthamiana (Tobacco). To evaluate the skipping efficiency of the p2a, f2a and f2a₃₀ sequences, tagrfp was connected to the 3′ end of the LRHK gene, encoding MM:NLS:CcaS(Δ23 A92V), via the three different 2A sequences and expressed from the P_{UBQ}-T_{rbcS} cassette. With perfect skipping, the TagRFP fluorescence should not be limited to the nucleus. With failed skipping, TagRFP would be fused with MM:NLS:CcaS(Δ23 A92V) and localized to the nucleus. As theoretical controls for perfect ribosomal skipping and complete failure of skipping, TagRFP and NLS:TagRFP was expressed from the P_{UBQ}-T_{rbcS} cassette. All three 2A sequences worked with high efficiency in planta (FIG. 9). The F2A₃₀ sequence was selected for further experiments.

### Testing the Highlighter System in Planta

### Photoswitching of the Highlighter System(s) in Response to Green Light, Blue Light and Darkness

The highlighter system was tested by transient transfection of Tobacco leaves. Agrobacterium tumefaciens (Agrobacterium), transformed with variants of the highlighter system, were used to infiltrate Tobacco leaves. The leaves were left to express the highlighter system for ˜2 days in the greenhouse before they received light treatments (blue light, green light or darkness) for minimum 18 hours (FIG. 10). For the light treatment the leaves were cut of the plant and kept in a humid environment inside plastic containers.

Light-controlled induction of YFP expression was evaluated by confocal imaging by analyzing and dividing the mean YFP fluorescence intensity by the mean RFP fluorescence intensity in the plant cell nuclei. As the YFP expression is inducible and the TagRFP expression is constitutive, a low ratio between the two signals can be interpreted as low target gene expression and a high ratio can be interpreted as a high target gene expression.

Four variants of the highlighter system were tested; Highlighter 209, Highlighter 210, Highlighter 213 and Highlighter 214 (Error! Reference source not found.). These systems test the importance of the A92V mutation (systems 209 and 213 have the A92V mutation, whereas 210 and 214 do not) and if it is better to add the NLS and VP64 domain to the N- or the C-terminus of CcaR (systems 209 and 210 are N-terminal fusions and 213 and 214 are C-terminal fusions).

The results revealed that for all constructs, blue light treatment reduced target gene expression compared to the green light treatment and the dark treatment. The largest fold-change in expression between light treatments were observed for Highlighter 213 and 214, where the VP64 domain and NLS are fused to the C-terminus of CcaR (Error! Reference source not found. 11).

### Second Test—RGB-White, Blue, Green, Red and Darkness

Next we evaluated the Highlighter systems 213 and 214 under more light regimes, this time including red light and RGB-white light. During expression of the system, while the leaves were still attached to the plant, the plants were grown in continuous blue light (FIG. 12).

In this experiment we include a NLS:Venus only control and a NLS:TagRFP only control. These two controls approximate the maximum (NLS:Venus only) and minimum ratios (NLS:TagRFP only) that can be achieved using our imaging system under the current experimental conditions and analysis methods. The systems, Highlighter 213 and Highlighter 214, were tested in duplicates.

In general, the systems are inactive under blue light conditions, intermediately active under green light and RGB-white light conditions and fully active under red light conditions and in the dark. The Highlighter system having the A92V mutation, Highlighter 213, exhibits broadly lower expression of the NLS:Venus target in the various light treatment regimes along with higher fold-change in expression between light treatments.

### Potential Applications for the Highlighter System

There is great demand for a chemical free, minimally invasive system for controlling target gene expression in plants. Such a tool would be of great value to both fundamental laboratory research as well as horticultural systems. With the highlighter system we have accomplished this and demonstrated its effectiveness in directing target gene expression in the plant host N. benthamiana. We will now continue to demonstrate its function in other model systems, including Arabidopsis thaliana and Medicago truncatula.

In plants, the availability of optogenetics tools are presently limited and Highlighter represents a major improvement over current technologies (e.g. cell-type specific promoters or chemical induction systems). Combined with laser-based light sources that offer high spatial- and temporal-resolution, the Highlighter system will enable research biologists to direct gene expression with unprecedented precision. Furthermore, light can be employed as a benign and low-cost regulator of gene expression, making it ideal for directing developmental and physiological changes in crop plants, compared to plant growth regulatory chemicals.

### Applications for the Highlighter System in Fundamental Research

Plant hosts, and potentially other eukaryotic hosts, expressing Highlighter can be reversibly directed to lower expression levels of a target gene using blue light treatment. This feature will allow biologists to examine the developmental and physiological responses of the organism to perturbation of nearly any biological process at the cell, tissue, organ, and organismal levels. Immediate interests include directing changes in the concentration of phytohormones. Examples below (Table 1).

TABLE 1
Precision genetics with the Highlighter system: Interrogating
consequences of spatiotemporal genetic perturbation.

| | | Basal | |
|---|---|---|---|
| Genetic background | Highlighter Target | expression | Blue light regime |
| Hormone | Biosynthetic gene | Elevated | Spatiotemporal |
| biosynthetic mutant | complement | hormone | depletion |
| Hormone | Catabolic gene | Depleted | Spatiotemporal |
| catabolic mutant | complement | hormone | elevation |

### Applications for the Highlighter System in Horticulture

Plant hosts expressing Highlighter can be directed to undergo key developmental transitions or physiological state changes through application of light treatments. The developed technology holds the potential to permit specific interventions for improved agronomic outcomes. Immediate interests include directing the timing of germination, flowering, senescence, drought tolerance, immune activation and synthetic metabolite production (i.e. use as ‘metabolic valve’). Examples below (Table 2).

TABLE 2
Precision horticulture with Highlighter: direct crop development
and physiology to suit agricultural/agropharmaceutical needs

| Genetic | | Blue light | Red light |
|---|---|---|---|
| background | Highlighter Target | regime | or basal |
| Flowering mutant | Floral regulator | Non-flowering | Synchronous |
| | complement | | flowering |
| Germination | Germination | Non- | Synchronous |
| mutant | regulator comple- | germinating | germination |
| | ment | | |
| Abscisic acid | Catabolic mutant | Induced | Low drought |
| (ABA) catabolic | complement | drought | tolerance/ |
| mutant | | tolerance | rapid growth |
| Salicylic acid | Biosynthetic | Reduced | Induction of |
| (SA) biosyn- | mutant comple- | biotroph | biotroph |
| thetic mutant | ment | immunity | immunity |
| Synthetic metab- | Synthetic | No | Synchronous |
| olite (e.g. phar- | metabolite | production of | production |
| maceutical) line | regulator com- | pharmaceutical | of pharmaceu- |
| lacking regulator | plement | | tical |

### Example 2

### Highlighter Response to Mixed Light Environments

Horticultural environments are typically mixed light environments, rather than monochromatic light. The responsiveness of the Highlighter system was therefore evaluated under light regimes where white light was enriched in either red (activating wavelengths) or blue light (inactivating wavelengths). Monochromatic red and blue light were used as control conditions to establish the maximum response for the system. In mixed light environments, a switch from white light with modest enrichment in red light to modest enrichment in blue light is sufficient to convert the Highlighter system 213 (tested in quadruplicate) from activation to inactivation of gene expression (FIG. 14).

### Creating Spectral Variants of the LRHK for Multichromatic Control of Gene Regulation

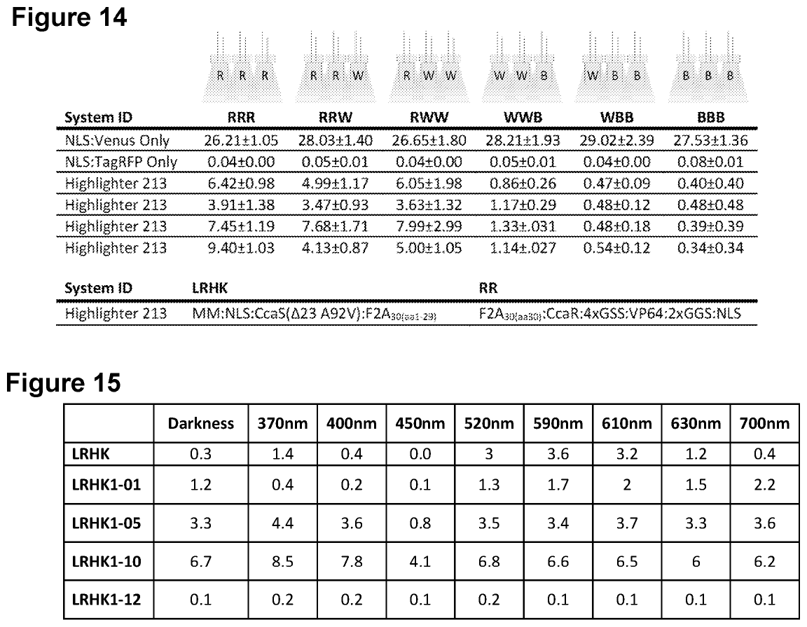

Advanced control of gene regulatory networks can be achieved by developing multichromatic optogenetic systems. We therefore tested if the LRHK we developed could be adapted to respond alternative light stimuli. A segment of the GAF domain in the LRHK (from the extreme N-terminal part of β1 sheet (DRV motif) to the C-terminal part of β6 sheet (WGL motif) was replaced by the corresponding segment of the following GAF domains; AnPixJg2, slr1393g2, NpR1597g4 and UirSg. The resulting LRHKs are referred to as LRHK1-01, LRHK1-05, LRHK1-10 and LRHK1-12, respectively. Gene induction (i.e. sfGFP fluorescence) downstream of the synthetic LRHKs were evaluated in response to darkness, ultraviolet light (370 nm and 400 nm), blue light (450 nm), green light (520 nm), yellow light (590 nm), orange light (610 nm), red light (630 nm), and far red light (700 nm) (FIG. 15).

The original LRHK is inactive in most light regimes, but strongly induces sfGFP expression in the green (520 nm), yellow (590 nm) and orange (610 nm) light regimes. In contrast, the LRHK1-01 induced sfGFP expression in all light regimes, except for the ultraviolet (370 nm and 400 nm) and blue (450 nm) light regimes. LRHK1-05 induced sfGFP expression in all light regimes, with the exception of blue light specifically. LRHK1-10 strongly induced sfGFP expression in all tested light regimes but still displays somewhat reduced induction of sfGFP expression in response to blue light (450 nm). LRHK1-12 is constitutively inactive in all light regimes. The results clearly demonstrate that the LRHK developed for the Highlighter system can be adapted to display new light responsive properties.

### Control of Gene Expression in Stably Transformed Arabidopsis in a Light Dependent Manner Using the Highlighter System

To demonstrate that the Highlighter system is able to control gene expression levels in stably transformed plants we attempted to complement the semi-dwarf phenotype of an Arabidopsis thaliana ga3ox1-3, ga3ox2-1 double mutant line that also expresses a nuclear localized GIBBERELLIN PERCEPTION SENSOR 1 (nGPS1) construct (ga3ox1-3, ga3ox2-1, nGPS1, Rizza 2017). Because the ga3ox2-1 mutant does not have a visible growth phenotype (Mitchum 2006), we hypothesized that AtGA3OX1 expression controlled by the Highlighter system could be used to complement the semi-dwarf phenotype in a light-dependent manner. A semi-dwarf phenotype of the ga3ox1-3, ga3ox2-1, nGPS1 line was clearly visible when grown in continuous blue-enriched white light and in continuous red-enriched white light. For the ga3ox1-3, ga3ox2-1, nGPS1 line transformed with the Highlighter system controlling AtGA3OX1 expression, the semi-dwarf phenotype is only observed when grown in ‘inactivating’ blue-enriched white light, whereas an undwarfed phenotype was observed in the same line grown in ‘activating’ red-enriched white light (FIG. 16). These results correspond well with the results observed in the transient tobacco experiments driving NLS:Venus expression under control of the Highlighter system.

### REFERENCES

1. Hirose, Y., Narikawa, R., Katayama, M. & Ikeuchi, M. Cyanobacteriochrome CcaS regulates phycoerythrin accumulation in Nostoc punctiforme, a group II chromatic adapter. Proc. Natl. Acad. Sci. 107, 8854-8859 (2010).
2. Schmidl, S. R., Sheth, R. U., Wu, A. & Tabor, J. J. Refactoring and optimization of light-switchable Escherichia coli two-component systems. ACS Synth. Biol. 3, 820-831 (2014).
3. Tabor, J. J., Levskaya, A. & Voigt, C. A. Multichromatic control of gene expression in Escherichia coli. J. Mol. Biol. 405, 315-324 (2011).
4. Mukougawa, K., Kanamoto, H., Kobayashi, T., Yokota, A. & Kohchi, T. Metabolic engineering to produce phytochromes with phytochromobilin, phycocyanobilin, or phycoerythrobilin chromophore in Escherichia coli. FEBS Lett. 580, 1333-1338 (2006).
5. Zuo, J., Niu, Q.-W. & Chua, N.-H. An estrogen-based transactivator XVE mediates highly inducible gene expression in transgenic plants. Plant J. 24, 265-273 (2000).
6. Huang, Q. et al. Distance and helical phase dependence of synergistic transcription activation in cis-regulatory module. PLoS One 7, 1-10 (2012).
7. Ochoa-Fernandez, R., Samodelov, S. L., Brandl, S. M., Wehinger, E., Muller, K., Weber, W., Zurbriggen, M. D., Optogenetics in Plants: Red/Far-Red Light Control of Gene Expression. Methods in Molecular Biology. 1408, 125-139 (2016).
8. Abe, K., Miyake, K., Nakamura, M., Kojima, K., Ferri, S., Ikebukuro, K., Sode, K. Engineering of a green-light inducible gene expression system in Synechocystis sp. PCC6803. Microbial Biotechnology. 7 (2) 177-183. (2013).
9. Hunter, P. Shining a light on optogenetics. EMBO Reports 17(5), 634-637 (2016).
10. Mitchum, M. G., Yamaguchi, S., Hanada, A., Kuwahara, A., Yoshioka, Y., Kato, T., Tabata, S., Kamiya, Y. & Sun, T.-P. Distinct and overlapping roles of two gibberellin 3-oxidases in Arabidopsis development. Plant J. 45(5), 804-818 (2006).
11. Rizza, A., Walia, A., Lanquar, V., Frommer, W. B. & Jones, A. M. In vivo gibberellin gradients visualized in rapidly elongating tissues. Nat Plants. 3(10), 803-813 (2017)

SEQUENCE LISTING
CcaS variants
SEQ ID NO: 1 CcaS (A92V); amino acid sequence
MGKFLIPIEFVFLAIAMTCYLWHRQNQERRRIEISIKQQTQRERF
INQITQHIRQSLNLETVLNTTVAEVKTLLQVDRVLIYRIWQDGTG
SVITESVNANYPSILGRTFSDEVFPVEYHQAYTKGKVRAINDIDQ
DDIEICLADFVKQFGVKSKLVVPILQHNRASSLDNESEFPYLWGL
LITHQCAFTRPWQPWEVELMKQLANQVAIAIQQSELYEQLQQLNK
DLENRVEKRTQQLAATNQSLRMEISERQKTEAALRHTNHTLQSLI
AASPRGIFTLNLADQIQIWNPTAERIFGWTETEIIAHPELLTSNI
LLEDYQQFKQKVLSGMVSPSLELKCQKKDGSWIEIVLSAAPLLDS
EENIAGLVAVVADITEQKRQAEQIRLLQSVVVNTNDAVVITEAEP
IDDPGPRILYVNEAFTKITGYTAEEMLGKTPRVLQGPKTSRTELD
RVRQAISQWQSVTVEVINYRKDGSEFWVEFSLVPVANKTGFYTHW
IAVQRDVTERRRTEEVRLALEREKELSRLKTRFFSMASHEFRTPL
STALAAAQLLENSEVAWLDPDKRSRNLHRIQNSVKNMVQLLDDIL
IINRAEAGKLEFNPNWLDLKLLFQQFIEEIQLSVSDQYYFDFICS
AQDTKALVDERLVRSILSNLLSNAIKYSPGGGQIKIALSLDSEQI
IFEVTDQGIGISPEDQKQIFEPFHRGKNVRNITGTGLGLMVAKKC
VDLHSGSILLKSAVDQGTTVTICLKRYNHLPRA
SEQ ID NO: 2 CcaS (A92V); nucleic acid
sequence
ATGGGCAAATTTCTAATTCCAATCGAATTTGTTTTTCTGGCGATC
GCCATGACCTGTTATTTATGGCACAGACAAAACCAAGAACGCCGC
AGGATTGAAATTAGCATCAAGCAACAAACCCAACGGGAACGATTT
ATTAACCAAATTACCCAACATATCCGCCAATCTTTAAACTTGGAA
ACGGTTTTAAATACCACCGTCGCTGAAGTTAAAACCCTGTTGCAA
GTTGATCGAGTTCTAATTTATCGCATTTGGCAAGATGGCACGGGC
AGCGTCATTACGGAATCGGTGAATGCCAATTATCCTAGTATTTTA
GGGCGGACCTTTTCCGATGAAGTTTTTCCCGTTGAATACCATCAA
GCCTACACCAAAGGTAAAGTACGGGCCATTAATGACATTGACCAG
GATGACATAGAGATTTGCCTAGCTGATTTCGTCAAACAATTTGGC
GTGAAATCAAAATTAGTAGTGCCCATTCTTCAACATAATCGTGCT
TCTTCCCTAGATAATGAATCAGAATTTCCCTATCTTTGGGGGCTG
TTAATTACCCATCAATGTGCTTTTACCCGGCCATGGCAACCGTGG
GAAGTGGAGTTAATGAAACAGCTAGCCAATCAGGTCGCGATCGCC
ATCCAACAATCGGAATTATATGAGCAATTACAGCAACTCAATAAA
GATTTGGAAAACCGAGTCGAAAAACGCACCCAGCAACTTGCCGCC
ACCAATCAATCCCTAAGAATGGAAATCAGTGAGCGACAAAAAACG
GAAGCCGCTCTCCGCCACACTAACCATACTCTGCAATCCCTGATT
GCGGCCTCCCCCAGGGGTATTTTTACCCTTAATTTAGCAGACCAA
ATTCAGATTTGGAATCCTACAGCAGAACGTATTTTTGGTTGGACA
GAAACAGAAATTATTGCCCATCCAGAATTATTAACATCCAACATT
TTGCTGGAAGATTATCAGCAATTTAAACAGAAAGTTTTATCAGGC
ATGGTTTCCCCTAGCCTAGAATTAAAATGTCAAAAAAAAGATGGT
AGTTGGATTGAAATTGTCCTTTCCGCTGCTCCCCTATTGGATAGT
GAAGAAAATATTGCCGGATTGGTGGCGGTTGTCGCCGATATTACC
GAGCAAAAGCGGCAGGCAGAACAAATTCGTTTGCTACAATCCGTT
GTGGTTAATACTAATGATGCGGTGGTGATTACGGAAGCGGAGCCC
ATTGATGATCCCGGGCCGAGAATTCTCTATGTCAATGAAGCATTT
ACTAAAATCACCGGTTATACTGCTGAAGAAATGCTAGGCAAAACC
CCCCGAGTTTTACAGGGACCAAAAACTAGTCGCACTGAATTAGAT
AGGGTGCGGCAAGCCATTAGTCAATGGCAATCAGTTACCGTTGAA
GTGATTAATTATCGTAAGGATGGCAGTGAGTTTTGGGTGGAATTT
AGTCTGGTGCCCGTTGCCAATAAAACAGGTTTTTACACCCATTGG
ATTGCTGTGCAAAGGGATGTCACTGAGCGCCGACGCACGGAGGAA
GTCCGCCTAGCTTTAGAACGGGAAAAAGAATTAAGCCGCCTAAAA
ACTCGTTTTTTCTCCATGGCTTCCCATGAATTTCGTACTCCCCTC
AGTACGGCCTTAGCTGCTGCCCAATTACTGGAAAATTCTGAAGTG
GCCTGGCTTGATCCCGATAAGCGTAGCCGGAACTTACACCGTATT
CAAAATTCCGTGAAAAATATGGTACAGCTCCTGGATGATATTTTA
ATCATTAACCGTGCCGAAGCGGGCAAATTGGAATTTAATCCTAAT
TGGTTAGATTTGAAATTATTGTTCCAGCAATTTATCGAAGAAATT
CAATTAAGTGTCAGTGACCAATATTATTTTGACTTTATTTGTAGC
GCTCAAGATACGAAGGCATTGGTGGATGAAAGGTTAGTGCGGTCT
ATTTTATCTAATCTGTTATCTAATGCGATTAAATACTCTCCCGGG
GGAGGGCAGATTAAAATTGCCCTAAGCCTAGATTCGGAACAGATT
ATTTTTGAAGTCACCGACCAGGGCATTGGCATTTCGCCAGAGGAC
CAAAAGCAAATTTTTGAACCCTTTCATCGGGGCAAAAATGTCAGA
AATATTACGGGAACAGGACTCGGTTTAATGGTTGCCAAGAAATGT
GTTGACTTACACAGTGGCAGTATCTTGCTAAAAAGTGCAGTTGAC
CAGGGAACAACAGTTACTATCTGTTTAAAACGCTATAACCATTTG
CCTCGAGCTTAG
SEQ ID NO: 3: M:NLS: CcaS (Δ23); amino acid
sequence
MLQPKKKRKVGGRQNQERRRIEISIKQQTQRERFINQITQHIRQS
LNLETVLNTTVAEVKTLLQVDRVLIYRIWQDGTGSAITESVNANY
PSILGRTFSDEVFPVEYHQAYTKGKVRAINDIDQDDIEICLADFV
KQFGVKSKLVVPILQHNRASSLDNESEFPYLWGLLITHQCAFTRP
WQPWEVELMKQLANQVAIAIQQSELYEQLQQLNKDLENRVEKRTQ
QLAATNQSLRMEISERQKTEAALRHTNHTLQSLIAASPRGIFTLN
LADQIQIWNPTAERIFGWTETEIIAHPELLTSNILLEDYQQFKQK
VLSGMVSPSLELKCQKKDGSWIEIVLSAAPLLDSEENIAGLVAVV
ADITEQKRQAEQIRLLQSVVVNTNDAWITEAEPIDDPGPRILYVN
EAFTKITGYTAEEMLGKTPRVLQGPKTSRTELDRVRQAISQWQSV
TVEVINYRKDGSEFWVEFSLVPVANKTGFYTHWIAVQRDVTERRR
TEEVRLALEREKELSRLKTRFFSMASHEFRTPLSTALAAAQLLEN
SEVAWLDPDKRSRNLHRIQNSVKNMVQLLDDILIINRAEAGKLEF
NPNWLDLKLLFQQFIEEIQLSVSDQYYFDFICSAQDTKALVDERL
VRSILSNLLSNAIKYSPGGGQIKIALSLDSEQIIFEVTDQGIGIS
PEDQKQIFEPFHRGKNVRNITGTGLGLMVAKKCVDLHSGSILLKS
AVDQGTTVTICLKRYNHLPRA
SEQ ID NO: 4 M:NLS:CcaS (Δ23); nucleic acid
sequence
ATGTTACAACCAAAGAAGAAAAGGAAGGTGGGTGGAAGACAAAAC
CAAGAACGCCGCAGGATTGAAATTAGCATCAAGCAACAAACCCAA
CGGGAACGATTTATTAACCAAATTACCCAACATATCCGCCAATCT
TTAAACTTGGAAACGGTTTTAAATACCACCGTCGCTGAAGTTAAA
ACCCTGTTGCAAGTTGATCGAGTTCTAATTTATCGCATTTGGCAA
GATGGCACGGGCAGCGCCATTACGGAATCGGTGAATGCCAATTAT
CCTAGTATTTTAGGGCGGACCTTTTCCGATGAAGTTTTTCCCGTT
GAATACCATCAAGCCTACACCAAAGGTAAAGTACGGGCCATTAAT
GACATTGACCAGGATGACATAGAGATTTGCCTAGCTGATTTCGTC
AAACAATTTGGCGTGAAATCAAAATTAGTAGTGCCCATTCTTCAA
CATAATCGTGCTTCTTCCCTAGATAATGAATCAGAATTTCCCTAT
CTTTGGGGGCTGTTAATTACCCATCAATGTGCTTTTACCCGGCCA
TGGCAACCGTGGGAAGTGGAGTTAATGAAACAGCTAGCCAATCAG
GTCGCGATCGCCATCCAACAATCGGAATTATATGAGCAATTACAG
CAACTCAATAAAGATTTGGAAAACCGAGTCGAAAAACGCACCCAG
CAACTTGCCGCCACCAATCAATCCCTAAGAATGGAAATCAGTGAG
CGACAAAAAACGGAAGCCGCTCTCCGCCACACTAACCATACTCTG
CAATCCCTGATTGCGGCCTCCCCCAGGGGTATTTTTACCCTTAAT
TTAGCAGACCAAATTCAGATTTGGAATCCTACAGCAGAACGTATT
TTTGGTTGGACAGAAACAGAAATTATTGCCCATCCAGAATTATTA
ACATCCAACATTTTGCTGGAAGATTATCAGCAATTTAAACAGAAA
GTTTTATCAGGCATGGTTTCCCCTAGCCTAGAATTAAAATGTCAA
AAAAAAGATGGTAGTTGGATTGAAATTGTCCTTTCCGCTGCTCCC
CTATTGGATAGTGAAGAAAATATTGCCGGATTGGTGGCGGTTGTC
GCCGATATTACCGAGCAAAAGCGGCAGGCAGAACAAATTCGTTTG
CTACAATCCGTTGTGGTTAATACTAATGATGCGGTGGTGATTACG
GAAGCGGAGCCCATTGATGATCCCGGGCCGAGAATTCTCTATGTC
AATGAAGCATTTACTAAAATCACCGGTTATACTGCTGAAGAAATG
CTAGGCAAAACCCCCCGAGTTTTACAGGGACCAAAAACTAGTCGC
ACTGAATTAGATAGGGTGCGGCAAGCCATTAGTCAATGGCAATCA
GTTACCGTTGAAGTGATTAATTATCGTAAGGATGGCAGTGAGTTT
TGGGTGGAATTTAGTCTGGTGCCCGTTGCCAATAAAACAGGTTTT
TACACCCATTGGATTGCTGTGCAAAGGGATGTCACTGAGCGCCGA
CGCACGGAGGAAGTCCGCCTAGCTTTAGAACGGGAAAAAGAATTA
AGCCGCCTAAAAACTCGTTTTTTCTCCATGGCTTCCCATGAATTT
CGTACTCCCCTCAGTACGGCCTTAGCTGCTGCCCAATTACTGGAA
AATTCTGAAGTGGCCTGGCTTGATCCCGATAAGCGTAGCCGGAAC
TTACACCGTATTCAAAATTCCGTGAAAAATATGGTACAGCTCCTG
GATGATATTTTAATCATTAACCGTGCCGAAGCGGGCAAATTGGAA
TTTAATCCTAATTGGTTAGATTTGAAATTATTGTTCCAGCAATTT
ATCGAAGAAATTCAATTAAGTGTCAGTGACCAATATTATTTTGAC
TTTATTTGTAGCGCTCAAGATACGAAGGCATTGGTGGATGAAAGG
TTAGTGCGGTCTATTTTATCTAATCTGTTATCTAATGCGATTAAA
TACTCTCCCGGGGGAGGGCAGATTAAAATTGCCCTAAGCCTAGAT
TCGGAACAGATTATTTTTGAAGTCACCGACCAGGGCATTGGCATT
TCGCCAGAGGACCAAAAGCAAATTTTTGAACCCTTTCATCGGGGC
AAAAATGTCAGAAATATTACGGGAACAGGACTCGGTTTAATGGTT
GCCAAGAAATGTGTTGACTTACACAGTGGCAGTATCTTGCTAAAA
AGTGCAGTTGACCAGGGAACAACAGTTACTATCTGTTTAAAACGC
TATAACCATTTGCCTCGAGCTTAG
SEQ ID NO: 5: CcaS (Δ 22 A92V); amino acid
sequence
MRQNQERRRIEISIKQQTQRERFINQITQHIRQSLNLETVLNTTV
AEVKTLLQVDRVLIYRIWQDGTGSVITESVNANYPSILGRTFSDE
VFPVEYHQAYTKGKVRAINDIDQDDIEICLADFVKQFGVKSKLVV
PILQHNRASSLDNESEFPYLWGLLITHQCAFTRPWQPWEVELMKQ
LANQVAIAIQQSELYEQLQQLNKDLENRVEKRTQQLAATNQSLRM
EISERQKTEAALRHTNHTLQSLIAASPRGIFTLNLADQIQIWNPT
AERIFGWTETEIIAHPELLTSNILLEDYQQFKQKVLSGMVSPSLE
LKCQKKDGSWIEIVLSAAPLLDSEENIAGLVAVVADITEQKRQAE
QIRLLQSWVNTNDAVVITEAEPIDDPGPRILYVNEAFTKITGYTA
EEMLGKTPRVLQGPKTSRTELDRVRQAISQWQSVTVEVINYRKDG
SEFVWEFSLVPVANKTGFYTHWIAVQRDVTERRRTEEVRLALERE
KELSRLKTRFFSMASHEFRTPLSTALAAAQLLENSEVAWLDPDKR
SRNLHRIQNSVKNMVQLLDDILIINRAEAGKLEFNPNWLDLKLLF
QQFIEEIQLSVSDQYYFDFICSAQDTKALVDERLVRSILSNLLSN
AIKYSPGGGQIKIALSLDSEQIIFEVTDQGIGISPEDQKQIFEPF
HRGKNVRNITGTGLGLMVAKKCVDLHSGSILLKSAVDQGTTVTIC
LKRYNHLPRA
SEQ ID NO: 6: CcaS (Δ 22 A92V); nucleic acid
sequence
ATGAGACAAAACCAAGAACGCCGCAGGATTGAAATTAGCATCAAG
CAACAAACCCAACGGGAACGATTTATTAACCAAATTACCCAACAT
ATCCGCCAATCTTTAAACTTGGAAACGGTTTTAAATACCACCGTC
GCTGAAGTTAAAACCCTGTTGCAAGTTGATCGAGTTCTAATTTAT
CGCATTTGGCAAGATGGCACGGGCAGCGTCATTACGGAATCGGTG
AATGCCAATTATCCTAGTATTTTAGGGCGGACCTTTTCCGATGAA
GTTTTTCCCGTTGAATACCATCAAGCCTACACCAAAGGTAAAGTA
CGGGCCATTAATGACATTGACCAGGATGACATAGAGATTTGCCTA
GCTGATTTCGTCAAACAATTTGGCGTGAAATCAAAATTAGTAGTG
CCCATTCTTCAACATAATCGTGCTTCTTCCCTAGATAATGAATCA
GAATTTCCCTATCTTTGGGGGCTGTTAATTACCCATCAATGTGCT
TTTACCCGGCCATGGCAACCGTGGGAAGTGGAGTTAATGAAACAG
CTAGCCAATCAGGTCGCGATCGCCATCCAACAATCGGAATTATAT
GAGCAATTACAGCAACTCAATAAAGATTTGGAAAACCGAGTCGAA
AAACGCACCCAGCAACTTGCCGCCACCAATCAATCCCTAAGAATG
GAAATCAGTGAGCGACAAAAAACGGAAGCCGCTCTCCGCCACACT
AACCATACTCTGCAATCCCTGATTGCGGCCTCCCCCAGGGGTATT
TTTACCCTTAATTTAGCAGACCAAATTCAGATTTGGAATCCTACA
GCAGAACGTATTTTTGGTTGGACAGAAACAGAAATTATTGCCCAT
CCAGAATTATTAACATCCAACATTTTGCTGGAAGATTATCAGCAA
TTTAAACAGAAAGTTTTATCAGGCATGGTTTCCCCTAGCCTAGAA
TTAAAATGTCAAAAAAAAGATGGTAGTTGGATTGAAATTGTCCTT
TCCGCTGCTCCCCTATTGGATAGTGAAGAAAATATTGCCGGATTG
GTGGCGGTTGTCGCCGATATTACCGAGCAAAAGCGGCAGGCAGAA
CAAATTCGTTTGCTACAATCCGTTGTGGTTAATACTAATGATGCG
GTGGTGATTACGGAAGCGGAGCCCATTGATGATCCCGGGCCGAGA
ATTCTCTATGTCAATGAAGCATTTACTAAAATCACCGGTTATACT
GCTGAAGAAATGCTAGGCAAAACCCCCCGAGTTTTACAGGGACCA
AAAACTAGTCGCACTGAATTAGATAGGGTGCGGCAAGCCATTAGT
CAATGGCAATCAGTTACCGTTGAAGTGATTAATTATCGTAAGGAT
GGCAGTGAGTTTTGGGTGGAATTTAGTCTGGTGCCCGTTGCCAAT
AAAACAGGTTTTTACACCCATTGGATTGCTGTGCAAAGGGATGTC
ACTGAGCGCCGACGCACGGAGGAAGTCCGCCTAGCTTTAGAACGG
GAAAAAGAATTAAGCCGCCTAAAAACTCGTTTTTTCTCCATGGCT
TCCCATGAATTTCGTACTCCCCTCAGTACGGCCTTAGCTGCTGCC
CAATTACTGGAAAATTCTGAAGTGGCCTGGCTTGATCCCGATAAG
CGTAGCCGGAACTTACACCGTATTCAAAATTCCGTGAAAAATATG
GTACAGCTCCTGGATGATATTTTAATCATTAACCGTGCCGAAGCG
GGCAAATTGGAATTTAATCCTAATTGGTTAGATTTGAAATTATTG
TTCCAGCAATTTATCGAAGAAATTCAATTAAGTGTCAGTGACCAA
TATTATTTTGACTTTATTTGTAGCGCTCAAGATACGAAGGCATTG
GTGGATGAAAGGTTAGTGCGGTCTATTTTATCTAATCTGTTATCT
AATGCGATTAAATACTCTCCCGGGGGAGGGCAGATTAAAATTGCC
CTAAGCCTAGATTCGGAACAGATTATTTTTGAAGTCACCGACCAG
GGCATTGGCATTTCGCCAGAGGACCAAAAGCAAATTTTTGAACCC
TTTCATCGGGGCAAAAATGTCAGAAATATTACGGGAACAGGACTC
GGTTTAATGGTTGCCAAGAAATGTGTTGACTTACACAGTGGCAGT
ATCTTGCTAAAAAGTGCAGTTGACCAGGGAACAACAGTTACTATC
TGTTTAAAACGCTATAACCATTTGCCTCGAGCTTAG
SEQ ID NO: 7 M:NLS: CcaS (Δ23 A92V); amino
acid sequence
MLQPKKKRKVGGRQNQERRRIEISIKQQTQRERFINQITQHIRQSL
NLETVLNTTVAEVKTLLQVDRVLIYRIWQDGTGSVITESVNANYPS
ILGRTFSDEVFPVEYHQAYTKGKVRAINDIDQDDIEICLADFVKQF
GVKSKLVVPILQHNRASSLDNESEFPYLWGLLITHQCAFTRPWQPW
EVELMKQIANQVAIAIQQSELYEQLQQLNKDLENRVEKRTQQLAAT
NQSLRMEISERQKTEAALRHTNHTLQSLIAASPRGIFTLNLADQIQ
IWNPTAERIFGWTETEIIAHPELLTSNILLEDYQQFKQKVLSGMVS
PSLELKCQKKDGSWIEIVLSAAPLLDSEENIAGLVAVVADITEQKR
QAEQIRLLQSVVVNTNDAVVITEAEPIDDPGPRILYVNEAFTKITG
YTAEEMLGKTPRVLQGPKTSRTELDRVRQAISQWQSVTVEVINYRK
DGSEFWVEFSLVPVANKTGFYTHWIAVQRDVTERRRTEEVRLALER
EKELSRLKTRFFSMASHEFRTPLSTALAAAQLLENSEVAWLDPDKR
SRNLHRIQNSVKNMVQLLDDILIINRAEAGKLEFNPNWLDLKLLFQ
QFIEEIQLSVSDQYYFDFICSAQDTKALVDERLVRSILSNLLSNAI
KYSPGGGQIKIALSLDSEQIIFEVTDQGIGISPEDQKQIFEPFHRG
KNVRNITGTGLGLMVAKKCVDLHSGSILLKSAVDQGTTVTICLKRY
NHLPRA
SEQ ID NO: 8 M:NLS: CcaS (Δ23 A92V); nucleic
acid sequence
ATGTTACAACCAAAGAAGAAAAGGAAGGTGGGTGGAAGACAAAACC
AAGAACGCCGCAGGATTGAAATTAGCATCAAGCAACAAACCCAACG
GGAACGATTTATTAACCAAATTACCCAACATATCCGCCAATCTTTA
AACTTGGAAACGGTTTTAAATACCACCGTCGCTGAAGTTAAAACCC
TGTTGCAAGTTGATCGAGTTCTAATTTATCGCATTTGGCAAGATGG
CACGGGCAGCGTCATTACGGAATCGGTGAATGCCAATTATCCTAGT
ATTTTAGGGCGGACCTTTTCCGATGAAGTTTTTCCCGTTGAATACC
ATCAAGCCTACACCAAAGGTAAAGTACGGGCCATTAATGACATTGA
CCAGGATGACATAGAGATTTGCCTAGCTGATTTCGTCAAACAATTT
GGCGTGAAATCAAAATTAGTAGTGCCCATTCTTCAACATAATCGTG
GCTTCTTCCCTAGATAATGAATCAGAATTTCCCTATCTTTGGGGCT
GTTAATTACCCATCAATGTGCTTTTACCCGGCCATGGCAACCGTGG
GAAGTGGAGTTAATGAAACAGCTAGCCAATCAGGTCGCGATCGCCA
TCCAACAATCGGAATTATATGAGCAATTACAGCAACTCAATAAAGA
TTTGGAAAACCGAGTCGAAAAACGCACCCAGCAACTTGCCGCCACC
AATCAATCCCTAAGAATGGAAATCAGTGAGCGACAAAAAACGGAAG
CCGCTCTCCGCCACACTAACCATACTCTGCAATCCCTGATTGCGGC
CTCCCCCAGGGGTATTTTTACCCTTAATTTAGCAGACCAAATTCAG
ATTTGGAATCCTACAGCAGAACGTATTTTTGGTTGGACAGAAACAG
AAATTATTGCCCATCCAGAATTATTAACATCCAACATTTTGCTGGA
AGATTATCAGCAATTTAAACAGAAAGTTTTATCAGGCATGGTTTCC
CCTAGCCTAGAATTAAAATGTCAAAAAAAAGATGGTAGTTGGATTG
AAATTGTCCTTTCCGCTGCTCCCCTATTGGATAGTGAAGAAAATAT
TGCCGGATTGGTGGCGGTTGTCGCCGATATTACCGAGCAAAAGCGG
CAGGCAGAACAAATTCGTTTGCTACAATCCGTTGTGGTTAATACTA
ATGATGCGGTGGTGATTACGGAAGCGGAGCCCATTGATGATCCCGG
GCCGAGAATTCTCTATGTCAATGAAGCATTTACTAAAATCACCGGT
TATACTGCTGAAGAAATGCTAGGCAAAACCCCCCGAGTTTTACAGG
GACCAAAAACTAGTCGCACTGAATTAGATAGGGTGCGGCAAGCCAT
TAGTCAATGGCAATCAGTTACCGTTGAAGTGATTAATTATCGTAAG
GATGGCAGTGAGTTTTGGGTGGAATTTAGTCTGGTGCCCGTTGCCA
ATAAAACAGGTTTTTACACCCATTGGATTGCTGTGCAAAGGGATGT
CACTGAGCGCCGACGCACGGAGGAAGTCCGCCTAGCTTTAGAACGG
GAAAAAGAATTAAGCCGCCTAAAAACTCGTTTTTTCTCCATGGCTT
CCCATGAATTTCGTACTCCCCTCAGTACGGCCTTAGCTGCTGCCCA
ATTACTGGAAAATTCTGAAGTGGCCTGGCTTGATCCCGATAAGCGT
AGCCGGAACTTACACCGTATTCAAAATTCCGTGAAAAATATGGTAC
AGCTCCTGGATGATATTTTAATCATTAACCGTGCCGAAGCGGGCAA
ATTGGAATTTAATCCTAATTGGTTAGATTTGAAATTATTGTTCCAG
CAATTTATCGAAGAAATTCAATTAAGTGTCAGTGACCAATATTATT
TTGACTTTATTTGTAGCGCTCAAGATACGAAGGCATTGGTGGATGA
AAGGTTAGTGCGGTCTATTTTATCTAATCTGTTATCTAATGCGATT
AAATACTCTCCCGGGGGAGGGCAGATTAAAATTGCCCTAAGCCTAG
ATTCGGAACAGATTATTTTTGAAGTCACCGACCAGGGCATTGGCAT
TTCGCCAGAGGACCAAAAGCAAATTTTTGAACCCTTTCATCGGGGC
AAAAATGTCAGAAATATTACGGGAACAGGACTCGGTTTAATGGTTG
CCAAGAAATGTGTTGACTTACACAGTGGCAGTATCTTGCTAAAAAG
TGCAGTTGACCAGGGAACAACAGTTACTATCTGTTTAAAACGCTAT
AACCATTTGCCTCGAGCTTAG
SEQ ID NO: 9 MM:NLS:CcaS(Δ23):F2A30(aa1-29)
amino acid sequence
MMLQPKKKRKVGGRQNQERRRIEISIKQQTQRERFINQITQHIRQS
LNLETVLNTTVAEVKTLLQVDRVLIYRIWQDGTGSAITESVNANYP
SILGRTFSDEVFPVEYHQAYTKGKVRAINDIDQDDIEICLADFVKQ
FGVKSKLVVPILQHNRASSLDNESEFPYLWGLLITHQCAFTRPWQP
WEVELMKQLANQVAIAIQQSELYEQLQQLNKDLENRVEKRTQQLAA
TNQSLRMEISERQKTEAALRHTNHTLQSLIAASPRGIFTLNLADQI
QIWNPTAERIFGWTETEIIAHPELLTSNILLEDYQQFKQKVLSGMV
SPSLELKCQKKDGSWIEIVLSAAPLLDSEENIAGLVAVVADITEQK
RQAEQIRLLQSVVVNTNDAVVITEAEPIDDPGPRILYVNEAFTKIT
GYTAEEMLGKTPRVLQGPKTSRTELDRVRQAISQWQSVTVEVINYR
KDGSEFWVEFSLVPVANKTGFYTHWIAVQRDVTERRRTEEVRLALE
REKELSRLKTRFFSMASHEFRTPLSTALAAAQLLENSEVAWLDPDK
RSRNLHRIQNSVKNMVQLLDDILIINRAEAGKLEFNPNWLDLKLLF
QQFIEEIQLSVSDQYYFDFICSAQDTKALVDERLVRSILSNLLSNA
IKYSPGGGQIKIALSLDSEQIIFEVTDQGIGISPEDQKQIFEPFHR
GKNVRNITGTGLGLMVAKKCVDLHSGSILLKSAVDQGTTVTICLKR
YNHLPRA
SEQ ID NO: 10 MM:NLS: CcaS(Δ23):F2A30
(aa1-29) nucleic acid sequence
ATGATGTTACAACCAAAGAAGAAAAGGAAGGTGGGTGGAAGACAGA
ACCAAGAACGAAGAAGAATAGAAATAAGTATCAAGCAGCAGACACA
ACGTGAGAGGTTTATCAACCAAATCACACAGCATATCAGACAATCT
CTTAATTTGGAGACTGTTTTGAACACTACAGTTGCTGAAGTTAAGA
CACTTTTGCAGGTTGATAGAGTTCTTATCTATAGAATCTGGCAAGA
TGGTACAGGATCTGCTATCACTGAGTCTGTTAATGCTAACTACCCT
TCTATTTTGGGTAGAACTTTTTCTGATGAGGTTTTCCCAGTTGAAT
ATCATCAAGCTTACACAAAGGGAAAAGTTAGAGCTATTAATGATAT
CGATCAGGATGATATCGAAATCTGTCTTGCTGATTTCGTTAAACAA
TTCGGTGTTAAGTCTAAACTTGTTGTTCCTATCTTGCAGCATAATA
GAGCTTCTTCTTTGGATAACGAATCTGAGTTTCCATATCTTTGGGG
ACTTTTGATTACACATCAGTGTGCTTTCACTAGACCTTGGCAACCT
TGGGAAGTTGAGCTTATGAAGCAGTTGGCTAACCAAGTTGCTATTG
CTATCCAACAGTCTGAGTTGTACGAACAACTTCAACAGTTGAATAA
GGATCTTGAGAACAGAGTTGAAAAAAGAACACAACAGTTGGCTGCT
ACTAATCAGTCTCTTAGGATGGAAATCTCTGAAAGACAAAAGACTG
AGGCTGCTTTGAGACATACTAACCATACACTTCAGTCTTTGATTGC
TGCTTCTCCTAGAGGTATCTTTACTCTTAATTTGGCTGATCAAATT
CAGATCTGGAACCCAACAGCTGAGCGAATCTTCGGATGGACTGAAA
CAGAGATTATCGCTCATCCTGAGCTTTTGACATCTAACATCCTTTT
GGAAGATTACCAACAGTTTAAGCAAAAGGTTCTTTCTGGTATGGTT
TCTCCATCTCTTGAGTTGAAGTGTCAGAAGAAAGATGGATCTTGGA
TTGAAATCGTTTTGTCTGCTGCTCCTCTTTTGGATTCTGAAGAGAA
CATTGCTGGTCTTGTTGCTGTTGTTGCTGATATCACTGAGCAAAAA
AGACAGGCTGAACAAATCAGACTTTTGCAATCTGTTGTTGTTAACA
CAAACGATGCTGTTGTTATTACTGAAGCTGAACCAATCGATGATCC
TGGACCAAGAATCCTTTATGTTAATGAGGCTTTCACTAAGATCACA
GGATACACTGCTGAAGAGATGTTGGGAAAGACTCCTAGAGTTCTTC
AAGGACCAAAAACTTCAAGAACTGAGTTGGATAGAGTTAGACAGGC
TATCTCTCAATGGCAGTCTGTTACAGTTGAAGTTATTAATTACAGA
AAGGATGGTTCTGAGTTTTGGGTTGAATTTTCTCTTGTTCCTGTTG
CTAACAAAACAGGATTTTACACTCATTGGATTGCTGTTCAAAGAGA
TGTTACAGAGAGAAGAAGAACTGAAGAGGTTAGACTTGCTTTGGAA
AGAGAGAAGGAACTTTCAAGATTGAAGACTAGATTTTTCTCTATGG
CTTCTCATGAGTTTAGAACACCACTTTCTACTGCTTTGGCTGCTGC
TCAACTTCTTGAAAATTCTGAAGTTGCTTGGCTTGATCCTGATAAG
AGATCAAGAAACCTTCATAGAATCCAAAATTCTGTTAAAAACATGG
TTCAACTTTTGGATGATATCTTGATTATCAACAGAGCTGAGGCTGG
AAAGCTTGAGTTTAATCCAAACTGGCTTGATTTGAAGCTTTTGTTC
CAACAGTTCATTGAAGAGATCCAGCTTTCTGTTTCTGATCAATACT
ACTTCGATTTCATCTGTTCTGCTCAAGATACTAAGGCTCTTGTTGA
TGAAAGATTGGTTAGATCTATCCTTTCTAATCTTTTGTCTAACGCT
ATCAAGTACTCTCCTGGAGGTGGACAGATTAAAATCGCTCTTTCTT
TGGATTCTGAGCAGATTATCTTCGAAGTTACAGATCAAGGTATTGG
AATCTCTCCTGAGGATCAAAAGCAGATCTTTGAACCATTCCATAGA
GGAAAGAATGTTAGAAACATTACTGGTACAGGACTTGGTTTGATGG
TTGCTAAGAAATGTGTTGATCTTCATTCTGGATCTATCCTTTTGAA
GTCTGCTGTGGATCAAGGAACAACTGTGACCATCTGTCTCAAAAGG
TACAACCATCTCCCAAGGGCT
SEQ ID NO: 11 MM:NLS:CcaS (Δ23 A92V):F2A30
(aa1-29) amino acid sequence
MMLQPKKKRKVGGRQNQERRRIEISIKQQTQRERFINQITQHIRQS
LNLETVLNTTVAEVKTLLQVDRVLIYRIWQDGTGSVITESVNANYP
SILGRTFSDEVFPVEYHQAYTKGKVRAINDIDQDDIEICLADFVKQ
FGVKSKLVVPILQHNRASSLDNESEFPYLWGLLITHQCAFTRPWQP
WEVELMKQLANQVAIAIQQSELYEQLQQLNKDLENRVEKRTQQLAA
TNQSLRMEISERQKTEAALRHTNHTLQSLIAASPRGIFTLNLADQI
QIWNPTAERIFGWTETEIIAHPELLTSNILLEDYQQFKQKVLSGMV
SPSLELKCQKKDGSWIEIVLSAAPLLDSEENIAGLVAVVADITEQK
RQAEQIRLLQSVVVNTNDAVVITEAEPIDDPGPRILYVNEAFTKIT
GYTAEEMLGKTPRVLQGPKTSRTELDRVRQAISQWQSVTVEVINYR
KDGSEFWVEFSLVPVANKTGFYTHWIAVQRDVTERRRTEEVRLALE
REKELSRLKTRFFSMASHEFRTPLSTALAAAQLLENSEVAWLDPDK
RSRNLHRIQNSVKNMVQLLDDILIINRAEAGKLEFNPNWLDLKLLF
QQFIEEIQLSVSDQYYFDFICSAQDTKALVDERLVRSILSNLLSNA
IKYSPGGGQIKIALSLDSEQIIFEVTDQGIGISPEDQKQIFEPFHR
GKNVRNITGTGLGLMVAKKCVDLHSGSILLKSAVDQGTTVTICLKR
YNHLPRA
SEQ ID NO: 12 MM:NLS:CcaS(Δ23 A92V):F2A30
(aa1-29) nucleic acid sequence
ATGATGTTACAACCAAAGAAGAAAAGGAAGGTGGGTGGAAGACAGA
ACCAAGAACGAAGAAGAATAGAAATAAGTATCAAGCAGCAGACACA
ACGTGAGAGGTTTATCAACCAAATCACACAGCATATCAGACAATCT
CTTAATTTGGAGACTGTTTTGAACACTACAGTTGCTGAAGTTAAGA
CACTTTTGCAGGTTGATAGAGTTCTTATCTATAGAATCTGGCAAGA
TGGTACAGGATCTGTTATCACTGAGTCTGTTAATGCTAACTACCCT
TCTATTTTGGGTAGAACTTTTTCTGATGAGGTTTTCCCAGTTGAAT
ATCATCAAGCTTACACAAAGGGAAAAGTTAGAGCTATTAATGATAT
CGATCAGGATGATATCGAAATCTGTCTTGCTGATTTCGTTAAACAA
TTCGGTGTTAAGTCTAAACTTGTTGTTCCTATCTTGCAGCATAATA
GAGCTTCTTCTTTGGATAACGAATCTGAGTTTCCATATCTTTGGGG
ACTTTTGATTACACATCAGTGTGCTTTCACTAGACCTTGGCAACCT
TGGGAAGTTGAGCTTATGAAGCAGTTGGCTAACCAAGTTGCTATTG
CTATCCAACAGTCTGAGTTGTACGAACAACTTCAACAGTTGAATAA
GGATCTTGAGAACAGAGTTGAAAAAAGAACACAACAGTTGGCTGCT
ACTAATCAGTCTCTTAGGATGGAAATCTCTGAAAGACAAAAGACTG
AGGCTGCTTTGAGACATACTAACCATACACTTCAGTCTTTGATTGC
TGCTTCTCCTAGAGGTATCTTTACTCTTAATTTGGCTGATCAAATT
CAGATCTGGAACCCAACAGCTGAGCGAATCTTCGGATGGACTGAAA
CAGAGATTATCGCTCATCCTGAGCTTTTGACATCTAACATCCTTTT
GGAAGATTACCAACAGTTTAAGCAAAAGGTTCTTTCTGGTATGGTT
TCTCCATCTCTTGAGTTGAAGTGTCAGAAGAAAGATGGATCTTGGA
TTGAAATCGTTTTGTCTGCTGCTCCTCTTTTGGATTCTGAAGAGAA
CATTGCTGGTCTTGTTGCTGTTGTTGCTGATATCACTGAGCAAAAA
AGACAGGCTGAACAAATCAGACTTTTGCAATCTGTTGTTGTTAACA
CAAACGATGCTGTTGTTATTACTGAAGCTGAACCAATCGATGATCC
TGGACCAAGAATCCTTTATGTTAATGAGGCTTTCACTAAGATCACA
GGATACACTGCTGAAGAGATGTTGGGAAAGACTCCTAGAGTTCTTC
AAGGACCAAAAACTTCAAGAACTGAGTTGGATAGAGTTAGACAGGC
TATCTCTCAATGGCAGTCTGTTACAGTTGAAGTTATTAATTACAGA
AAGGATGGTTCTGAGTTTTGGGTTGAATTTTCTCTTGTTCCTGTTG
CTAACAAAACAGGATTTTACACTCATTGGATTGCTGTTCAAAGAGA
TGTTACAGAGAGAAGAAGAACTGAAGAGGTTAGACTTGCTTTGGAA
AGAGAGAAGGAACTTTCAAGATTGAAGACTAGATTTTTCTCTATGG
CTTCTCATGAGTTTAGAACACCACTTTCTACTGCTTTGGCTGCTGC
TCAACTTCTTGAAAATTCTGAAGTTGCTTGGCTTGATCCTGATAAG
AGATCAAGAAACCTTCATAGAATCCAAAATTCTGTTAAAAACATGG
TTCAACTTTTGGATGATATCTTGATTATCAACAGAGCTGAGGCTGG
AAAGCTTGAGTTTAATCCAAACTGGCTTGATTTGAAGCTTTTGTTC
CAACAGTTCATTGAAGAGATCCAGCTTTCTGTTTCTGATCAATACT
ACTTCGATTTCATCTGTTCTGCTCAAGATACTAAGGCTCTTGTTGA
TGAAAGATTGGTTAGATCTATCCTTTCTAATCTTTTGTCTAACGCT
ATCAAGTACTCTCCTGGAGGTGGACAGATTAAAATCGCTCTTTCTT
TGGATTCTGAGCAGATTATCTTCGAAGTTACAGATCAAGGTATTGG
AATCTCTCCTGAGGATCAAAAGCAGATCTTTGAACCATTCCATAGA
GGAAAGAATGTTAGAAACATTACTGGTACAGGACTTGGTTTGATGG
TTGCTAAGAAATGTGTTGATCTTCATTCTGGATCTATCCTTTTGAA
GTCTGCTGTGGATCAAGGAACAACTGTGACCATCTGTCTCAAAAGG
TACAACCATCTCCCAAGGGCT
CcaR variants
SEQ ID NO: 13: F2A30(aa30):NLS:2xGGS:VP64:
4xGGS:Cca Ramino acid
PGSLQPKKKRKVGGGGSGGSDALDDFDLDMLGSDALDDFDLDMLGS
DALDDFDLDMLGSDALDDFDLDMLGGSGGSGGSGGSMRILLVEDDL
PLAETLAEALSDQLYTVDIATDASLAWDYASRLEYDLVILDVMLPE
LDGITLCQKWRSHSYLMPILMMTARDTINDKITGLDAGADDYVVKP
VDLGELFARVRALLRRGCATCQPVLEWGPIRLDPSTYEVSYDNEVL
SLTRKEYSILELLLRNGRRVLSRSMIIDSIWKLESPPEEDTVKVHV
RSLRQKLKSAGLSADAIETVHGIGYRLANLTEKSLCQGKN
SEQ ID NO: 14: F2A30(aa30):NLS:2xGGS:VP64:
4xGGS:CcaR nucleic acid
CCAGGTTCACTCCAGCCTAAGAAGAAGAGAAAGGTTGGAGGTGGTG
GCTCCGGAGGCTCTGATGCCCTCGACGATTTCGACCTCGATATGCT
CGGTTCTGATGCTCTCGATGACTTTGACCTTGACATGCTTGGATCA
GACGCTTTGGACGACTTCGACTTGGACATGTTGGGATCTGATGCAC
TTGATGATTTTGACCTTGATATGCTTGGTGGTTCAGGAGGGTCTGG
TGGATCAGGAGGATCTATGAGAATACTCCTCGTGGAAGATGATTTG
CCATTAGCAGAAACCCTCGCAGAAGCTTTGTCTGATCAACTTTACA
CTGTTGATATTGCTACAGATGCTTCTTTGGCTTGGGATTATGCTTC
TAGACTTGAATACGATTTGGTTATTCTTGATGTTATGTTGCCTGAG
CTTGATGGAATTACTCTTTGTCAGAAGTGGAGATCTCATTCTTATT
TGATGCCAATCCTTATGATGACTGCTAGAGATACAATTAATGATAA
GATCACAGGACTTGATGCTGGTGCTGATGATTACGTTGTTAAACCT
GTTGATTTGGGTGAACTTTTTGCTAGAGTTAGAGCTCTTTTGAGAA
GAGGATGTGCTACTTGTCAACCAGTTTTGGAGTGGGGTCCTATTAG
ACTTGATCCATCTACTTATGAAGTTTCTTACGATAATGAGGTTTTG
TCTCTTACAAGAAAGGAATACTCTATCTTGGAGCTTTTGCTTAGAA
ACGGAAGAAGAGTTCTTTCTAGATCTATGATCATCGATTCTATCTG
GAAGTTGGAGTCTCCTCCAGAAGAGGATACAGTTAAAGTTCATGTT
AGATCTTTGAGACAAAAGCTTAAGTCTGCTGGACTTTCTGCTGATG
CTATTGAAACTGTTCATGGAATCGGTTACAGATTGGCTAATCTTAC
AGAGAAGTCTTTGTGTCAGGGAAAGAAT
SEQ ID NO: 15: F2A30(aa30):CcaR:4xGSS:VP64:
2xGGS:NLS amino acid
PMRILLVEDDLPLAETLAEALSDQLYTVDIATDASLAWDYASRLEY
DLVILDVMLPELDGITLCQKWRSHSYLMPILMMTARDTINDKITGL
DAGADDYVVKPVDLGELFARVRALLRRGCATCQPVLEWGPIRLDPS
DTYEVSYDNEVLSLTRKEYSILELLLRNGRRVLSRSMIISIWKLES
PPEEDTVKVHVRSLRQKLKSAGLSADAIETVHGIGYRLANLTEKSL
NCQGKGGSGGSGGSGGSDALDDFDLDMLGSDALDDFDLDMLGSDAL
DDFDLDMLGSDALDDFDLDMLGGSGGSLQPKKKRKVGG
SEQ ID NO: 16: F2A30(aa30):CcaR:4xGSS:VP64:
2xGGS:NLS nucleic acid
CCAATGAGAATACTCCTCGTGGAAGATGATTTGCCATTAGCAGAAA
CCCTCGCAGAAGCTTTGTCTGATCAACTTTACACTGTTGATATTGC
TACAGATGCTTCTTTGGCTTGGGATTATGCTTCTAGACTTGAATAC
GATTTGGTTATTCTTGATGTTATGTTGCCTGAGCTTGATGGAATTA
CTCTTTGTCAGAAGTGGAGATCTCATTCTTATTTGATGCCAATCCT
TATGATGACTGCTAGAGATACAATTAATGATAAGATCACAGGACTT
GATGCTGGTGCTGATGATTACGTTGTTAAACCTGTTGATTTGGGTG
AACTTTTTGCTAGAGTTAGAGCTCTTTTGAGAAGAGGATGTGCTAC
TTGTCAACCAGTTTTGGAGTGGGGTCCTATTAGACTTGATCCATCT
TCACTTATGAAGTTTCTTACGATAATGAGGTTTTGTCTTACAAGAA
AGGAATACTCTATCTTGGAGCTTTTGCTTAGAAACGGAAGAAGAGT
TCTTTCTAGATCTATGATCATCGATTCTATCTGGAAGTTGGAGTCT
CCTCCAGAAGAGGATACAGTTAAAGTTCATGTTAGATCTTTGAGAC
AAAAGCTTAAGTCTGCTGGACTTTCTGCTGATGCTATTGAAACTGT
TCATGGAATCGGTTACAGATTGGCTAATCTTACAGAGAAGTCTTTG
TGTCAGGGAAAGAATGGAGGCTCCGGTGGGTCAGGTGGTTCTGGAG
GCTCGGATGCCCTCGACGATTTCGACCTCGATATGCTCGGTTCTGA
TGCTCTCGATGACTTTGACCTTGACATGCTTGGATCAGACGCTTTG
GACGACTTCGACTTGGACATGTTGGGATCTGATGCACTTGATGATT
TTGACCTTGATATGCTTGGCGGTTCCGGTGGATCACTCCAGCCTAA
GAAGAAGAGAAAGGTTGGAGGT
Synthetic plant promoter and cognate
transcription activator SEQ ID NO: 17:
CTTTCCGATTTCTTTACGATTTCCGCTTTCCGATTTCTTTACGATT
TGGCTTTCCGATTTCTTTACGATTTATCCTTCGCAAGACCCTTCCT
CTATATAAGGAAGTTCATTTCATTTGGAGAGGA
SEQ ID NO: 40; ccaR CRE motif
CTTTCCGATTTCTTTACGATTT
SEQ ID NO: 41; P35Smin(-51)
CTTCGCAAGACCCTTCCTCTATATAAGGAAGTTCATTTCATTTGGA
GAGGA)
SEQ ID NO: 42: Terminator sequence (Trbcs)
AGCTTTCGTTCGTATCATCGGTTTCGACAACGTTCGTCAAGTTCAA
TGCATCAGTTTCATTGCGCACACACCAGAATCCTACTGAGTTtGAG
TATTATGGCATTGGGAAAacTGTTTTTCTTGTACCATTTGTTGTGC
TTGTAATTTACTGTGTTTTTTATTCGGTTTTCGCTATCGAACTGTG
AAATGGAAATGGATGGAGAAGAGTTAATGAATGATATGGTCCTTTT
GTTCATTCTCAAATTAATATTATTTGTTTTTTCTCTTATTTGTTGT
GTGTTGAATTTGAAAtTATAAGAGATATGCAAACATTTTGTTTTGA
GTAAAAATGTGTCAAATCGTGGCCTCTAATGACCGAAGTTAATATG
AGGAGTAAAACACTTGTAGTTGTACCATTATGCTTATTCACTAGGC
AACAAATATATTTTCAGACCTAGAAAAGCTGCAAATGTTACTGAAT
ACAAGTATGTCCTCTTGTGTTTTAGACATTTATGAACTTTCCTTTA
TGTAATTTTCCAGAATCCTTGTCAGATTCTAATCATTGCTTTATAA
TTATAGTTATACTCATGGATTTGTAGTTGAGTATGAAAATATTTTT
TAATGCATTTTATGACTTGCCAATTGATTGACAACATGCATCAaTC
G
SEQ ID NO: 43: Terminator sequence (NOS
terminator):
TAGAGTAGATGCCGACCGAACAAGAGCTGATTTCGAGAACGCCTCA
GCCAGCAACTCGCGCGAGCCTAGCAAGGCAAATGCGAGAGAACGGC
CTTACGCTTGGTGGCACAGTTCTCGTCCACAGTTCGCTAAGCTCGC
TCGGCTGGGTCGCGGGAGGGCCGGTCGCAGTGATTCAGGAATTAAT
TCCCTAGAGTCAAGCAGATCGTTCAAACATTTGGCAATAAAGTTTC
TTAAGATTGAATCCTGTTGCCGGTCTTGCGATGATTATCATATAAT
TTCTGTTGAATTACGTTAAGCATGTAATAATTAACATGTAATGCAT
GACGTTATTTATGAGATGGGTTTTTATGATTAGAGTCCCGCAATTA
TACATTTAATACGCGATAGAAAACAAAATATAGCGCGCAAACTAGG
ATAAATTATCGCGCGCGGTGTCATCTATGTTACTAGATCGACCGGC
ATGCAAGCTGAT
SEQ ID NO: 44 UBQ10 promoter
ACCCGACGAGtCAGTAATAAACGGCGTCAAAGTGGTTGCAGCCGGC
ACACACGAGTCGTGTTTATCAACTCAAAGCACAAATACTTTTCCTC
AACCTAAAAATAAGGCAATTAGCCAAAAACAACTTTGCGTGTAAAC
AACGCTCAATACACGTGTCATTTTATTATTAGCTATTGCTTCACCG
CCTTAGCTTTCTCGTGACCTAGTCGTCCTCGTCTTTTCTTCTTCTT
CTTCTATAAAACAATACCCAAAGAGCTCTTCTTCTTCACAATTCAG
ATTTCAATTTCTCAAAATCTTAAAAACTTTCTCTCAATTCTCTCTA
CCGTGATCAAGGTAAATTTCTGTGTTCCTTATTCTCTCAAAATCTT
CGATTTTGTTTTCGTTCGATCCCAATTTCGTATATGTTCTTTGGTT
TAGATTCTGTTAATCTTAGATCGAAGACGATTTTCTGGGTTTGATC
GTTAGATATCATCTTAATTCTCGATTAGGGTTTCATAGATATCATC
CGATTTGTTCAAATAATTTGAGTTTTGTCGAATAATTACTCTTCGA
TTTGTGATTTCTATCTAGATCTGGTGTTAGTTTCTAGTTTGTGCGA
TCGAATTTGTAGATTAATCTGAGTTTTTCTGATTAACAGCTCGAGT
GCGGGATC
SEQ ID NO: 47 LRHK1-01 nucleic acid sequence
ATGATGTTACAACCAAAGAAGAAAAGGAAGGTGGGTGGAAGACAAA
ACCAAGAACGCCGCAGGATTGAAATTAGCATCAAGCAACAAACCCA
ACGGGAACGATTTATTAACCAAATTACCCAACATATCCGCCAATCT
TTAAACTTGGAAACGGTTTTAAATACCACCGTCGCTGAAGTTAAAA
CCCTGTTGCAAGTTGATCGAGTTGCCGTGTACCGTTTTAACCCGGA
TTGGAGCGGCGAGTTTGTGGCCGAAAGCGTGGGTAGCGGTTGGGTG
AAACTGGTGGGCCCGGATATCAAAACCGTGTGGGAAGACACACATC
TGCAAGAAACCCAAGGTGGTCGCTATCGCCATCAAGAAAGCTTCGT
GGTGAACGACATTTATGAGGCCGGCCATTTCAGCTGCCATCTGGAG
ATTTTAGAACAGTTTGAAATTAAAGCCTACATTATCGTGCCGGTTT
TTGCCGCCGAAAAACTGTGGGGTTTACTGGCCGCCTATCAGAACAG
TGGTACCCGCGAATGGGTGGAATGGGAAAGCAGCTTTCTGACCCAA
GTTGGTCTGCAGTTCGGCATCGCCATCCAACAATCGGAATTATATG
AGCAATTACAGCAACTCAATAAAGATTTGGAAAACCGAGTCGAAAA
ACGCACCCAGCAACTTGCCGCCACCAATCAATCCCTAAGAATGGAA
ATCAGTGAGCGACAAAAAACGGAAGCCGCTCTCCGCCACACTAACC
ATACTCTGCAATCCCTGATTGCGGCCTCCCCCAGGGGTATTTTTAC
CCTTAATTTAGCAGACCAAATTCAGATTTGGAATCCTACAGCAGAA
CGTATTTTTGGTTGGACAGAAACAGAAATTATTGCCCATCCAGAAT
TATTAACATCCAACATTTTGCTGGAAGATTATCAGCAATTTAAACA
GAAAGTTTTATCAGGCATGGTTTCCCCTAGCCTAGAATTAAAATGT
CAAAAAAAAGATGGTAGTTGGATTGAAATTGTCCTTTCCGCTGCTC
CCCTATTGGATAGTGAAGAAAATATTGCCGGATTGGTGGCGGTTGT
CGCCGATATTACCGAGCAAAAGCGGCAGGCAGAACAAATTCGTTTG
CTACAATCCGTTGTGGTTAATACTAATGATGCGGTGGTGATTACGG
AAGCGGAGCCCATTGATGATCCCGGGCCGAGAATTCTCTATGTCAA
TGAAGCATTTACTAAAATCACCGGTTATACTGCTGAAGAAATGCTA
GGCAAAACCCCCCGAGTTTTACAGGGACCAAAAACTAGTCGCACTG
AATTAGATAGGGTGCGGCAAGCCATTAGTCAATGGCAATCAGTTAC
CGTTGAAGTGATTAATTATCGTAAGGATGGCAGTGAGTTTTGGGTG
GAATTTAGTCTGGTGCCCGTTGCCAATAAAACAGGTTTTTACACCC
ATTGGATTGCTGTGCAAAGGGATGTCACTGAGCGCCGACGCACGGA
GGAAGTCCGCCTAGCTTTAGAACGGGAAAAAGAATTAAGCCGCCTA
AAAACTCGTTTTTTCTCCATGGCTTCCCATGAATTTCGTACTCCCC
TCAGTACGGCCTTAGCTGCTGCCCAATTACTGGAAAATTCTGAAGT
GGCCTGGCTTGATCCCGATAAGCGTAGCCGGAACTTACACCGTATT
CAAAATTCCGTGAAAAATATGGTACAGCTCCTGGATGATATTTTAA
TCATTAACCGTGCCGAAGCGGGCAAATTGGAATTTAATCCTAATTG
GTTAGATTTGAAATTATTGTTCCAGCAATTTATCGAAGAAATTCAA
TTAAGTGTCAGTGACCAATATTATTTTGACTTTATTTGTAGCGCTC
AAGATACGAAGGCATTGGTGGATGAAAGGTTAGTGCGGTCTATTTT
ATCTAATCTGTTATCTAATGCGATTAAATACTCTCCCGGGGGAGGG
CAGATTAAAATTGCCCTAAGCCTAGATTCGGAACAGATTATTTTTG
AAGTCACCGACCAGGGCATTGGCATTTCGCCAGAGGACCAAAAGCA
AATTTTTGAACCCTTTCATCGGGGCAAAAATGTCAGAAATATTACG
GGAACAGGACTCGGTTTAATGGTTGCCAAGAAATGTGTTGACTTAC
ACAGTGGCAGTATCTTGCTAAAAAGTGCAGTTGACCAGGGAACAAC
AGTTACTATCTGTTTAAAACGCTATAACCATTTGCCTCGAGCTCAC
AAACAGAAAATTGTGGCACCGGTGAAGCAGACTCTCAACTTTGACT
TGCTAAAGTTAGCTGGTGATGTTGAATCTAATCCTGGA
SEQ ID NO: 48 LRHK1-05 nucleic acid sequence
ATGATGTTACAACCAAAGAAGAAAAGGAAGGTGGGTGGAAGACAAA
ACCAAGAACGCCGCAGGATTGAAATTAGCATCAAGCAACAAACCCA
ACGGGAACGATTTATTAACCAAATTACCCAACATATCCGCCAATCT
TTAAACTTGGAAACGGTTTTAAATACCACCGTCGCTGAAGTTAAAA
CCCTGTTGCAAGTTGATCGAGTTCTGGTGTATCGCTTTAACCCGGA
TTGGAGCGGCGAGTTTATCCATGAAAGCGTGGCCCAGATGTGGGAA
CCGCTGAAGGATCTGCAGAACAACTTTCCGCTGTGGCAAGATACCT
ATTTACAAGAAAATGAGGGTGGCCGCTACCGCAATCATGAAAGTCT
GGCCGTGGGCGATGTGGAAACCGCCGGTTTCACCGATTGCCATTTA
GATAATCTGCGTCGCTTCGAAATTCGCGCCTTTCTGACCGTGCCGG
TTTTTGTTGGTGAACAGCTGTGGGGTCTGCTGGGCGCCTATCAGAA
TGGTGCACCGCGCCATTGGCAAGCTCGCGAAATTCATCTGCTGCAC
CAGATCGCCAACCAGCTGGGTATCGCCATCCAACAATCGGAATTAT
ATGAGCAATTACAGCAACTCAATAAAGATTTGGAAAACCGAGTCGA
AAAACGCACCCAGCAACTTGCCGCCACCAATCAATCCCTAAGAATG
GAAATCAGTGAGCGACAAAAAACGGAAGCCGCTCTCCGCCACACTA
ACCATACTCTGCAATCCCTGATTGCGGCCTCCCCCAGGGGTATTTT
TACCCTTAATTTAGCAGACCAAATTCAGATTTGGAATCCTACAGCA
GAACGTATTTTTGGTTGGACAGAAACAGAAATTATTGCCCATCCAG
AATTATTAACATCCAACATTTTGCTGGAAGATTATCAGCAATTTAA
ACAGAAAGTTTTATCAGGCATGGTTTCCCCTAGCCTAGAATTAAAA
TGTCAAAAAAAAGATGGTAGTTGGATTGAAATTGTCCTTTCCGCTG
CTCCCCTATTGGATAGTGAAGAAAATATTGCCGGATTGGTGGCGGT
TGTCGCCGATATTACCGAGCAAAAGCGGCAGGCAGAACAAATTCGT
TTGCTACAATCCGTTGTGGTTAATACTAATGATGCGGTGGTGATTA
CGGAAGCGGAGCCCATTGATGATCCCGGGCCGAGAATTCTCTATGT
CAATGAAGCATTTACTAAAATCACCGGTTATACTGCTGAAGAAATG
CTAGGCAAAACCCCCCGAGTTTTACAGGGACCAAAAACTAGTCGCA
CTGAATTAGATAGGGTGCGGCAAGCCATTAGTCAATGGCAATCAGT
TACCGTTGAAGTGATTAATTATCGTAAGGATGGCAGTGAGTTTTGG
GTGGAATTTAGTCTGGTGCCCGTTGCCAATAAAACAGGTTTTTACA
CCCATTGGATTGCTGTGCAAAGGGATGTCACTGAGCGCCGACGCAC
GGAGGAAGTCCGCCTAGCTTTAGAACGGGAAAAAGAATTAAGCCGC
CTAAAAACTCGTTTTTTCTCCATGGCTTCCCATGAATTTCGTACTC
CCCTCAGTACGGCCTTAGCTGCTGCCCAATTACTGGAAAATTCTGA
AGTGGCCTGGCTTGATCCCGATAAGCGTAGCCGGAACTTACACCGT
ATTCAAAATTCCGTGAAAAATATGGTACAGCTCCTGGATGATATTT
TAATCATTAACCGTGCCGAAGCGGGCAAATTGGAATTTAATCCTAA
TTGGTTAGATTTGAAATTATTGTTCCAGCAATTTATCGAAGAAATT
CAATTAAGTGTCAGTGACCAATATTATTTTGACTTTATTTGTAGCG
CTCAAGATACGAAGGCATTGGTGGATGAAAGGTTAGTGCGGTCTAT
TTTATCTAATCTGTTATCTAATGCGATTAAATACTCTCCCGGGGGA
GGGCAGATTAAAATTGCCCTAAGCCTAGATTCGGAACAGATTATTT
TTGAAGTCACCGACCAGGGCATTGGCATTTCGCCAGAGGACCAAAA
GCAAATTTTTGAACCCTTTCATCGGGGCAAAAATGTCAGAAATATT
ACGGGAACAGGACTCGGTTTAATGGTTGCCAAGAAATGTGTTGACT
TACACAGTGGCAGTATCTTGCTAAAAAGTGCAGTTGACCAGGGAAC
AACAGTTACTATCTGTTTAAAACGCTATAACCATTTGCCTCGAGCT
CACAAACAGAAAATTGTGGCACCGGTGAAGCAGACTCTCAACTTTG
ACTTGCTAAAGTTAGCTGGTGATGTTGAATCTAATCCTGGA
SEQ ID NO: 49 LRHK1-10 nucleic acid sequence
ATGATGTTACAACCAAAGAAGAAAAGGAAGGTGGGTGGAAGACAAA
ACCAAGAACGCCGCAGGATTGAAATTAGCATCAAGCAACAAACCCA
ACGGGAACGATTTATTAACCAAATTACCCAACATATCCGCCAATCT
TTAAACTTGGAAACGGTTTTAAATACCACCGTCGCTGAAGTTAAAA
CCCTGTTGCAAGTTGATCGAGTTACCATTTATCGTTTTCGCGCCGA
TTGGAGCGGTGAATTTGTGGCCGAATCTTTAGCCCAAGGTTGGACA
CCGGTGCGTGAAATTGTGCCGGTGGTTGCCGATGACTATCTGCAAG
AAACCCAAGGTCGCAACTTTGCCAATGGCAAAAGCATCGTGATTAA
AGATATTTACAGCGCCAACTACAGCATCTGCCACATTGCACTGCTG
GAACTGATGCAAGCTCGCGCCTATATGATCGTGCCGATCTTCCAAG
GTGAAAAGCTGTGGGGTCTGCTGGCCGCCTATCAGAACATCAAGCC
TCGCGATTGGCAAGAAGATGAGGTGGATCTGGTGATGCAGATCGGT
ACCCAGCTGGGCATCGCCATCCAACAATCGGAATTATATGAGCAAT
TACAGCAACTCAATAAAGATTTGGAAAACCGAGTCGAAAAACGCAC
CCAGCAACTTGCCGCCACCAATCAATCCCTAAGAATGGAAATCAGT
GAGCGACAAAAAACGGAAGCCGCTCTCCGCCACACTAACCATACTC
TGCAATCCCTGATTGCGGCCTCCCCCAGGGGTATTTTTACCCTTAA
TTTAGCAGACCAAATTCAGATTTGGAATCCTACAGCAGAACGTATT
TTTGGTTGGACAGAAACAGAAATTATTGCCCATCCAGAATTATTAA
CATCCAACATTTTGCTGGAAGATTATCAGCAATTTAAACAGAAAGT
TTTATCAGGCATGGTTTCCCCTAGCCTAGAATTAAAATGTCAAAAA
AAAGATGGTAGTTGGATTGAAATTGTCCTTTCCGCTGCTCCCCTAT
TGGATAGTGAAGAAAATATTGCCGGATTGGTGGCGGTTGTCGCCGA
TATTACCGAGCAAAAGCGGCAGGCAGAACAAATTCGTTTGCTACAA
TCCGTTGTGGTTAATACTAATGATGCGGTGGTGATTACGGAAGCGG
AGCCCATTGATGATCCCGGGCCGAGAATTCTCTATGTCAATGAAGC
ATTTACTAAAATCACCGGTTATACTGCTGAAGAAATGCTAGGCAAA
ACCCCCCGAGTTTTACAGGGACCAAAAACTAGTCGCACTGAATTAG
ATAGGGTGCGGCAAGCCATTAGTCAATGGCAATCAGTTACCGTTGA
AGTGATTAATTATCGTAAGGATGGCAGTGAGTTTTGGGTGGAATTT
AGTCTGGTGCCCGTTGCCAATAAAACAGGTTTTTACACCCATTGGA
TTGCTGTGCAAAGGGATGTCACTGAGCGCCGACGCACGGAGGAAGT
CCGCCTAGCTTTAGAACGGGAAAAAGAATTAAGCCGCCTAAAAACT
CGTTTTTTCTCCATGGCTTCCCATGAATTTCGTACTCCCCTCAGTA
CGGCCTTAGCTGCTGCCCAATTACTGGAAAATTCTGAAGTGGCCTG
GCTTGATCCCGATAAGCGTAGCCGGAACTTACACCGTATTCAAAAT
TCCGTGAAAAATATGGTACAGCTCCTGGATGATATTTTAATCATTA
ACCGTGCCGAAGCGGGCAAATTGGAATTTAATCCTAATTGGTTAGA
TTTGAAATTATTGTTCCAGCAATTTATCGAAGAAATTCAATTAAGT
GTCAGTGACCAATATTATTTTGACTTTATTTGTAGCGCTCAAGATA
CGAAGGCATTGGTGGATGAAAGGTTAGTGCGGTCTATTTTATCTAA
TCTGTTATCTAATGCGATTAAATACTCTCCCGGGGGAGGGCAGATT
AAAATTGCCCTAAGCCTAGATTCGGAACAGATTATTTTTGAAGTCA
CCGACCAGGGCATTGGCATTTCGCCAGAGGACCAAAAGCAAATTTT
TGAACCCTTTCATCGGGGCAAAAATGTCAGAAATATTACGGGAACA
GGACTCGGTTTAATGGTTGCCAAGAAATGTGTTGACTTACACAGTG
GCAGTATCTTGCTAAAAAGTGCAGTTGACCAGGGAACAACAGTTAC
TATCTGTTTAAAACGCTATAACCATTTGCCTCGAGCTCACAAACAG
AAAATTGTGGCACCGGTGAAGCAGACTCTCAACTTTGACTTGCTAA
AGTTAGCTGGTGATGTTGAATCTAATCCTGGA
SEQ ID NO: 50 LRHK1-12 nucleic acid sequence
ATGATGTTACAACCAAAGAAGAAAAGGAAGGTGGGTGGAAGACAAA
ACCAAGAACGCCGCAGGATTGAAATTAGCATCAAGCAACAAACCCA
ACGGGAACGATTTATTAACCAAATTACCCAACATATCCGCCAATCT
TTAAACTTGGAAACGGTTTTAAATACCACCGTCGCTGAAGTTAAAA
CCCTGTTGCAAGTTGATCGAGTTGTTATTTTTCAGTTTTCACCCGA
CTCTGACTTTTCCGTTGGTAATATTGTGGCAGAGTCGGTATTGGCT
CCATTTAAGCCAATCATTAATAGTGCAATTGAAGAAACTTGTTTTA
GTAATAACTATGCCCAAAGGTATCAGCAGGGCAGAATTCAGGTCAT
TGAGGATATTCACCAGTCCCATCTTAGGCAATGCCACATTGACTTT
CTTGCCAGGCTACAGGTCAGGGCAAACCTAGTGCTACCACTAATTA
ATGATGCCATTTTGTGGGGCTTATTGTGTATTCATCAATGTGACAG
TTCTAGAGTTTGGGAACAAACAGAAATTGATCTGCTCAAGCAGATC
ACTAATCAGTTTGAAATCGCCATCCAACAATCGGAATTATATGAGC
AATTACAGCAACTCAATAAAGATTTGGAAAACCGAGTCGAAAAACG
CACCCAGCAACTTGCCGCCACCAATCAATCCCTAAGAATGGAAATC
AGTGAGCGACAAAAAACGGAAGCCGCTCTCCGCCACACTAACCATA
CTCTGCAATCCCTGATTGCGGCCTCCCCCAGGGGTATTTTTACCCT
TAATTTAGCAGACCAAATTCAGATTTGGAATCCTACAGCAGAACGT
ATTTTTGGTTGGACAGAAACAGAAATTATTGCCCATCCAGAATTAT
TAACATCCAACATTTTGCTGGAAGATTATCAGCAATTTAAACAGAA
AGTTTTATCAGGCATGGTTTCCCCTAGCCTAGAATTAAAATGTCAA
AAAAAAGATGGTAGTTGGATTGAAATTGTCCTTTCCGCTGCTCCCC
TATTGGATAGTGAAGAAAATATTGCCGGATTGGTGGCGGTTGTCGC
CGATATTACCGAGCAAAAGCGGCAGGCAGAACAAATTCGTTTGCTA
CAATCCGTTGTGGTTAATACTAATGATGCGGTGGTGATTACGGAAG
CGGAGCCCATTGATGATCCCGGGCCGAGAATTCTCTATGTCAATGA
AGCATTTACTAAAATCACCGGTTATACTGCTGAAGAAATGCTAGGC
AAAACCCCCCGAGTTTTACAGGGACCAAAAACTAGTCGCACTGAAT
TAGATAGGGTGCGGCAAGCCATTAGTCAATGGCAATCAGTTACCGT
TGAAGTGATTAATTATCGTAAGGATGGCAGTGAGTTTTGGGTGGAA
TTTAGTCTGGTGCCCGTTGCCAATAAAACAGGTTTTTACACCCATT
GGATTGCTGTGCAAAGGGATGTCACTGAGCGCCGACGCACGGAGGA
AGTCCGCCTAGCTTTAGAACGGGAAAAAGAATTAAGCCGCCTAAAA
ACTCGTTTTTTCTCCATGGCTTCCCATGAATTTCGTACTCCCCTCA
GTACGGCCTTAGCTGCTGCCCAATTACTGGAAAATTCTGAAGTGGC
CTGGCTTGATCCCGATAAGCGTAGCCGGAACTTACACCGTATTCAA
AATTCCGTGAAAAATATGGTACAGCTCCTGGATGATATTTTAATCA
TTAACCGTGCCGAAGCGGGCAAATTGGAATTTAATCCTAATTGGTT
AGATTTGAAATTATTGTTCCAGCAATTTATCGAAGAAATTCAATTA
AGTGTCAGTGACCAATATTATTTTGACTTTATTTGTAGCGCTCAAG
ATACGAAGGCATTGGTGGATGAAAGGTTAGTGCGGTCTATTTTATC
TAATCTGTTATCTAATGCGATTAAATACTCTCCCGGGGGAGGGCAG
ATTAAAATTGCCCTAAGCCTAGATTCGGAACAGATTATTTTTGAAG
TCACCGACCAGGGCATTGGCATTTCGCCAGAGGACCAAAAGCAAAT
TTTTGAACCCTTTCATCGGGGCAAAAATGTCAGAAATATTACGGGA
ACAGGACTCGGTTTAATGGTTGCCAAGAAATGTGTTGACTTACACA
GTGGCAGTATCTTGCTAAAAAGTGCAGTTGACCAGGGAACAACAGT
TACTATCTGTTTAAAACGCTATAACCATTTGCCTCGAGCTCACAAA
CAGAAAATTGTGGCACCGGTGAAGCAGACTCTCAACTTTGACTTGC
TAAAGTTAGCTGGTGATGTTGAATCTAATCCTGGA

1. A nucleic acid construct comprising a nucleic acid encoding a light-responsive histidine kinase and/or a nucleic acid encoding a response regulator, wherein the nucleic acid encodes a light-responsive histidine kinase as defined in any one of SEQ ID NOs: 1, 3, 5, 7, 9 or 11 or a functional variant thereof and wherein the response regulator encodes a response regulator as defined in SEQ ID NO 13 or 15 or a functional variant thereof.

2. The nucleic acid construct of claim 1, wherein the nucleic acid encoding a light-responsive histidine kinase comprises or consists of SEQ ID NO 2, 4, 6, 8, 10 or 12 or a functional variant thereof or comprises or consists of SEQ ID NO: 47, 48, 49 or 50 or a functional variant thereof and wherein the nucleic acid encoding a response regulator comprises or consists of SEQ ID NO: 14 or 16 or a functional variant thereof.

3. (canceled)

4. The nucleic acid construct of claim 1, wherein the construct comprises at least one regulatory sequence operably linked to at least one of the light-responsive histidine kinase and the response regulator, wherein the regulatory sequence is a constitutive promoter.

5-11. (canceled)

12. The nucleic acid construct of claim 1, wherein the construct further comprises a target sequence operably linked to a regulatory sequence that is specifically activated by the response regulator.

13. The nucleic acid construct of claim 12, wherein the regulatory sequence comprises a nucleic acid sequence as defined in SEQ ID NO: 17 or a functional variant thereof.

14-15. (canceled)

16. A host cell comprising the nucleic acid construct of claim 1.

17. The host cell of claim 16, wherein the cell is a eukaryotic or prokaryotic cell.

18. (canceled)

19. A transgenic organism expressing the nucleic acid construct of claim 1.

20. (canceled)

21. A method of producing a transgenic organism as defined in claim 19, the method comprising:
a. selecting a part of the organism;
b. transfecting at least one cell of the part of the organism of part (a) with the nucleic acid construct of claim 1; and
c. regenerating at least one organism derived from the transfected cell or cells.

22-23. (canceled)

24. A method of modulating expression of a target gene in an organism the method comprising introducing and expressing a nucleic acid construct as defined in claim 1 in said organism and applying at least one wavelength of light, wherein preferably said wavelength of light activates or represses activation of a LRHK.

25. A method of modulating any biochemical response in an organism, the method comprising introducing and expressing at least one nucleic acid construct as defined in claim 1 in said organism and applying at least wavelength of light, wherein preferably said wavelength of light activates or represses activation of a LRHK.

26. (canceled)

27. The method of claim 24 wherein expression of a target gene can be increased or decreased by applying at least one first wavelength of light.

28. The method of claim 27, wherein expression of a target gene can be decreased or increased or further decreased or increased by applying at least one second wavelength of light, wherein the first wavelength of light is different from the second wavelength of light.

29. The method of claim 24, wherein the wavelength of light may have one of the following ranges, 430 to 495 nm (blue light), 495 to 570 nm (green light), 600 to 750 nm (red light), white light or white light enriched with at least one of red, blue or green light or wherein the wavelength of light is dark light (no visible light).

30. (canceled)

31. The method of claim 27, wherein the first wavelength of light that increases expression of the target gene is preferably green, white or red light or is white light enriched with red light and wherein the first wavelength of light that decreases expression of the target gene is preferably blue light or is white light enriched with blue light.

32. (canceled)

33. The method of claim 28, wherein the second wavelength of light that further increases expression of a target gene is red light, and wherein the second wavelength of light that decreases expression of a target gene is blue light.

34-35. (canceled)

36. A photoreceptor molecule comprising a phytochrome and a chromophore, wherein the phytochrome comprises an amino acid sequence as defined in any of SEQ ID NOs 1, 3, 5, 7, 9 and 11.

37. The photoreceptor molecule of claim 36, wherein the chromophore is selected from PCB (phycocyanobilin), PφB (phytochromobilin) and BV (biliverdin).

38-40. (canceled)

41. A nucleic acid construct comprising a target sequence operably linked to a regulatory sequence, wherein the regulatory sequence is a regulatory sequence that is specifically activated by the response regulator, wherein the regulatory sequence comprises a nucleic acid sequence as defined in SEQ ID NO: 17 or a functional variant thereof.

42. (canceled)

43. A nucleic acid comprising:
a. a nucleic acid sequence encoding a polypeptide as defined in any of SEQ ID NOs 1, 3, 5, 7, 9, 11, 13 and 15;
b. a nucleic acid sequence as defined in any of SEQ ID NOs 2, 4, 6, 8, 10, 12, 14, 16, 17, 47, 48, 49 or 50 or the complementary sequence thereof;
c. a nucleic acid with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the nucleic acid sequence of (a) or (b); or
d. a nucleic acid sequence that is capable of hybridising under stringent conditions as defined herein to the nucleic acid sequence of any of (a) to (c).


SEQUENCE LISTING (50 sequences)

SEQ ID NO: 1 - protein, 753 aa, Artificial Sequence
  CcaS (A92V)
MGKFLIPIEFVFLAIAMTCYLWHRQNQERRRIEISIKQQTQRERFINQITQHIRQSLNLETVLNTTVAEVKTLLQVDRVLIYRIWQDGTGSVITESVNANYPSILGRTFSDEVFPVEYHQAYTKGKVRAINDIDQDDIEICLADFVKQFGVKSKLVVPILQHNRASSLDNESEFPYLWGLLITHQCAFTRPWQPWEVELMKQLANQVAIAIQQSELYEQLQQLNKDLENRVEKRTQQLAATNQSLRMEISERQKTEAALRHTNHTLQSLIAASPRGIFTLNLADQIQIWNPTAERIFGWTETEIIAHPELLTSNILLEDYQQFKQKVLSGMVSPSLELKCQKKDGSWIEIVLSAAPLLDSEENIAGLVAVVADITEQKRQAEQIRLLQSVVVNTNDAVVITEAEPIDDPGPRILYVNEAFTKITGYTAEEMLGKTPRVLQGPKTSRTELDRVRQAISQWQSVTVEVINYRKDGSEFWVEFSLVPVANKTGFYTHWIAVQRDVTERRRTEEVRLALEREKELSRLKTRFFSMASHEFRTPLSTALAAAQLLENSEVAWLDPDKRSRNLHRIQNSVKNMVQLLDDILIINRAEAGKLEFNPNWLDLKLLFQQFIEEIQLSVSDQYYFDFICSAQDTKALVDERLVRSILSNLLSNAIKYSPGGGQIKIALSLDSEQIIFEVTDQGIGISPEDQKQIFEPFHRGKNVRNITGTGLGLMVAKKCVDLHSGSILLKSAVDQGTTVTICLKRYNHLPRA

SEQ ID NO: 2 - DNA, 2262 bp, Artificial Sequence
  CcaS (A92V)
atgggcaaatttctaattccaatcgaatttgtttttctggcgatcgccatgacctgttatttatggcacagacaaaaccaagaacgccgcaggattgaaattagcatcaagcaacaaacccaacgggaacgatttattaaccaaattacccaacatatccgccaatctttaaacttggaaacggttttaaataccaccgtcgctgaagttaaaaccctgttgcaagttgatcgagttctaatttatcgcatttggcaagatggcacgggcagcgtcattacggaatcggtgaatgccaattatcctagtattttagggcggaccttttccgatgaagtttttcccgttgaataccatcaagcctacaccaaaggtaaagtacgggccattaatgacattgaccaggatgacatagagatttgcctagctgatttcgtcaaacaatttggcgtgaaatcaaaattagtagtgcccattcttcaacataatcgtgcttcttccctagataatgaatcagaatttccctatctttgggggctgttaattacccatcaatgtgcttttacccggccatggcaaccgtgggaagtggagttaatgaaacagctagccaatcaggtcgcgatcgccatccaacaatcggaattatatgagcaattacagcaactcaataaagatttggaaaaccgagtcgaaaaacgcacccagcaacttgccgccaccaatcaatccctaagaatggaaatcagtgagcgacaaaaaacggaagccgctctccgccacactaaccatactctgcaatccctgattgcggcctcccccaggggtatttttacccttaatttagcagaccaaattcagatttggaatcctacagcagaacgtatttttggttggacagaaacagaaattattgcccatccagaattattaacatccaacattttgctggaagattatcagcaatttaaacagaaagttttatcaggcatggtttcccctagcctagaattaaaatgtcaaaaaaaagatggtagttggattgaaattgtcctttccgctgctcccctattggatagtgaagaaaatattgccggattggtggcggttgtcgccgatattaccgagcaaaagcggcaggcagaacaaattcgtttgctacaatccgttgtggttaatactaatgatgcggtggtgattacggaagcggagcccattgatgatcccgggccgagaattctctatgtcaatgaagcatttactaaaatcaccggttatactgctgaagaaatgctaggcaaaaccccccgagttttacagggaccaaaaactagtcgcactgaattagatagggtgcggcaagccattagtcaatggcaatcagttaccgttgaagtgattaattatcgtaaggatggcagtgagttttgggtggaatttagtctggtgcccgttgccaataaaacaggtttttacacccattggattgctgtgcaaagggatgtcactgagcgccgacgcacggaggaagtccgcctagctttagaacgggaaaaagaattaagccgcctaaaaactcgttttttctccatggcttcccatgaatttcgtactcccctcagtacggccttagctgctgcccaattactggaaaattctgaagtggcctggcttgatcccgataagcgtagccggaacttacaccgtattcaaaattccgtgaaaaatatggtacagctcctggatgatattttaatcattaaccgtgccgaagcgggcaaattggaatttaatcctaattggttagatttgaaattattgttccagcaatttatcgaagaaattcaattaagtgtcagtgaccaatattattttgactttatttgtagcgctcaagatacgaaggcattggtggatgaaaggttagtgcggtctattttatctaatctgttatctaatgcgattaaatactctcccgggggagggcagattaaaattgccctaagcctagattcggaacagattatttttgaagtcaccgaccagggcattggcatttcgccagaggaccaaaagcaaatttttgaaccctttcatcggggcaaaaatgtcagaaatattacgggaacaggactcggtttaatggttgccaagaaatgtgttgacttacacagtggcagtatcttgctaaaaagtgcagttgaccagggaacaacagttactatctgtttaaaacgctataaccatttgcctcgagcttag

SEQ ID NO: 3 - protein, 742 aa, Artificial Sequence
  CcaS (23)
MLQPKKKRKVGGRQNQERRRIEISIKQQTQRERFINQITQHIRQSLNLETVLNTTVAEVKTLLQVDRVLIYRIWQDGTGSAITESVNANYPSILGRTFSDEVFPVEYHQAYTKGKVRAINDIDQDDIEICLADFVKQFGVKSKLVVPILQHNRASSLDNESEFPYLWGLLITHQCAFTRPWQPWEVELMKQLANQVAIAIQQSELYEQLQQLNKDLENRVEKRTQQLAATNQSLRMEISERQKTEAALRHTNHTLQSLIAASPRGIFTLNLADQIQIWNPTAERIFGWTETEIIAHPELLTSNILLEDYQQFKQKVLSGMVSPSLELKCQKKDGSWIEIVLSAAPLLDSEENIAGLVAVVADITEQKRQAEQIRLLQSVVVNTNDAVVITEAEPIDDPGPRILYVNEAFTKITGYTAEEMLGKTPRVLQGPKTSRTELDRVRQAISQWQSVTVEVINYRKDGSEFWVEFSLVPVANKTGFYTHWIAVQRDVTERRRTEEVRLALEREKELSRLKTRFFSMASHEFRTPLSTALAAAQLLENSEVAWLDPDKRSRNLHRIQNSVKNMVQLLDDILIINRAEAGKLEFNPNWLDLKLLFQQFIEEIQLSVSDQYYFDFICSAQDTKALVDERLVRSILSNLLSNAIKYSPGGGQIKIALSLDSEQIIFEVTDQGIGISPEDQKQIFEPFHRGKNVRNITGTGLGLMVAKKCVDLHSGSILLKSAVDQGTTVTICLKRYNHLPRA

SEQ ID NO: 4 - DNA, 2229 bp, Artificial Sequence
  MNLS CcaS (23)
atgttacaaccaaagaagaaaaggaaggtgggtggaagacaaaaccaagaacgccgcaggattgaaattagcatcaagcaacaaacccaacgggaacgatttattaaccaaattacccaacatatccgccaatctttaaacttggaaacggttttaaataccaccgtcgctgaagttaaaaccctgttgcaagttgatcgagttctaatttatcgcatttggcaagatggcacgggcagcgccattacggaatcggtgaatgccaattatcctagtattttagggcggaccttttccgatgaagtttttcccgttgaataccatcaagcctacaccaaaggtaaagtacgggccattaatgacattgaccaggatgacatagagatttgcctagctgatttcgtcaaacaatttggcgtgaaatcaaaattagtagtgcccattcttcaacataatcgtgcttcttccctagataatgaatcagaatttccctatctttgggggctgttaattacccatcaatgtgcttttacccggccatggcaaccgtgggaagtggagttaatgaaacagctagccaatcaggtcgcgatcgccatccaacaatcggaattatatgagcaattacagcaactcaataaagatttggaaaaccgagtcgaaaaacgcacccagcaacttgccgccaccaatcaatccctaagaatggaaatcagtgagcgacaaaaaacggaagccgctctccgccacactaaccatactctgcaatccctgattgcggcctcccccaggggtatttttacccttaatttagcagaccaaattcagatttggaatcctacagcagaacgtatttttggttggacagaaacagaaattattgcccatccagaattattaacatccaacattttgctggaagattatcagcaatttaaacagaaagttttatcaggcatggtttcccctagcctagaattaaaatgtcaaaaaaaagatggtagttggattgaaattgtcctttccgctgctcccctattggatagtgaagaaaatattgccggattggtggcggttgtcgccgatattaccgagcaaaagcggcaggcagaacaaattcgtttgctacaatccgttgtggttaatactaatgatgcggtggtgattacggaagcggagcccattgatgatcccgggccgagaattctctatgtcaatgaagcatttactaaaatcaccggttatactgctgaagaaatgctaggcaaaaccccccgagttttacagggaccaaaaactagtcgcactgaattagatagggtgcggcaagccattagtcaatggcaatcagttaccgttgaagtgattaattatcgtaaggatggcagtgagttttgggtggaatttagtctggtgcccgttgccaataaaacaggtttttacacccattggattgctgtgcaaagggatgtcactgagcgccgacgcacggaggaagtccgcctagctttagaacgggaaaaagaattaagccgcctaaaaactcgttttttctccatggcttcccatgaatttcgtactcccctcagtacggccttagctgctgcccaattactggaaaattctgaagtggcctggcttgatcccgataagcgtagccggaacttacaccgtattcaaaattccgtgaaaaatatggtacagctcctggatgatattttaatcattaaccgtgccgaagcgggcaaattggaatttaatcctaattggttagatttgaaattattgttccagcaatttatcgaagaaattcaattaagtgtcagtgaccaatattattttgactttatttgtagcgctcaagatacgaaggcattggtggatgaaaggttagtgcggtctattttatctaatctgttatctaatgcgattaaatactctcccgggggagggcagattaaaattgccctaagcctagattcggaacagattatttttgaagtcaccgaccagggcattggcatttcgccagaggaccaaaagcaaatttttgaaccctttcatcggggcaaaaatgtcagaaatattacgggaacaggactcggtttaatggttgccaagaaatgtgttgacttacacagtggcagtatcttgctaaaaagtgcagttgaccagggaacaacagttactatctgtttaaaacgctataaccatttgcctcgagcttag

SEQ ID NO: 5 - protein, 731 aa, Artificial Sequence
  CcaS (22 A92V)
MRQNQERRRIEISIKQQTQRERFINQITQHIRQSLNLETVLNTTVAEVKTLLQVDRVLIYRIWQDGTGSVITESVNANYPSILGRTFSDEVFPVEYHQAYTKGKVRAINDIDQDDIEICLADFVKQFGVKSKLVVPILQHNRASSLDNESEFPYLWGLLITHQCAFTRPWQPWEVELMKQLANQVAIAIQQSELYEQLQQLNKDLENRVEKRTQQLAATNQSLRMEISERQKTEAALRHTNHTLQSLIAASPRGIFTLNLADQIQIWNPTAERIFGWTETEIIAHPELLTSNILLEDYQQFKQKVLSGMVSPSLELKCQKKDGSWIEIVLSAAPLLDSEENIAGLVAVVADITEQKRQAEQIRLLQSVVVNTNDAVVITEAEPIDDPGPRILYVNEAFTKITGYTAEEMLGKTPRVLQGPKTSRTELDRVRQAISQWQSVTVEVINYRKDGSEFWVEFSLVPVANKTGFYTHWIAVQRDVTERRRTEEVRLALEREKELSRLKTRFFSMASHEFRTPLSTALAAAQLLENSEVAWLDPDKRSRNLHRIQNSVKNMVQLLDDILIINRAEAGKLEFNPNWLDLKLLFQQFIEEIQLSVSDQYYFDFICSAQDTKALVDERLVRSILSNLLSNAIKYSPGGGQIKIALSLDSEQIIFEVTDQGIGISPEDQKQIFEPFHRGKNVRNITGTGLGLMVAKKCVDLHSGSILLKSAVDQGTTVTICLKRYNHLPRA

SEQ ID NO: 6 - DNA, 2196 bp, Artificial Sequence
  CcaS (22 A92V)
atgagacaaaaccaagaacgccgcaggattgaaattagcatcaagcaacaaacccaacgggaacgatttattaaccaaattacccaacatatccgccaatctttaaacttggaaacggttttaaataccaccgtcgctgaagttaaaaccctgttgcaagttgatcgagttctaatttatcgcatttggcaagatggcacgggcagcgtcattacggaatcggtgaatgccaattatcctagtattttagggcggaccttttccgatgaagtttttcccgttgaataccatcaagcctacaccaaaggtaaagtacgggccattaatgacattgaccaggatgacatagagatttgcctagctgatttcgtcaaacaatttggcgtgaaatcaaaattagtagtgcccattcttcaacataatcgtgcttcttccctagataatgaatcagaatttccctatctttgggggctgttaattacccatcaatgtgcttttacccggccatggcaaccgtgggaagtggagttaatgaaacagctagccaatcaggtcgcgatcgccatccaacaatcggaattatatgagcaattacagcaactcaataaagatttggaaaaccgagtcgaaaaacgcacccagcaacttgccgccaccaatcaatccctaagaatggaaatcagtgagcgacaaaaaacggaagccgctctccgccacactaaccatactctgcaatccctgattgcggcctcccccaggggtatttttacccttaatttagcagaccaaattcagatttggaatcctacagcagaacgtatttttggttggacagaaacagaaattattgcccatccagaattattaacatccaacattttgctggaagattatcagcaatttaaacagaaagttttatcaggcatggtttcccctagcctagaattaaaatgtcaaaaaaaagatggtagttggattgaaattgtcctttccgctgctcccctattggatagtgaagaaaatattgccggattggtggcggttgtcgccgatattaccgagcaaaagcggcaggcagaacaaattcgtttgctacaatccgttgtggttaatactaatgatgcggtggtgattacggaagcggagcccattgatgatcccgggccgagaattctctatgtcaatgaagcatttactaaaatcaccggttatactgctgaagaaatgctaggcaaaaccccccgagttttacagggaccaaaaactagtcgcactgaattagatagggtgcggcaagccattagtcaatggcaatcagttaccgttgaagtgattaattatcgtaaggatggcagtgagttttgggtggaatttagtctggtgcccgttgccaataaaacaggtttttacacccattggattgctgtgcaaagggatgtcactgagcgccgacgcacggaggaagtccgcctagctttagaacgggaaaaagaattaagccgcctaaaaactcgttttttctccatggcttcccatgaatttcgtactcccctcagtacggccttagctgctgcccaattactggaaaattctgaagtggcctggcttgatcccgataagcgtagccggaacttacaccgtattcaaaattccgtgaaaaatatggtacagctcctggatgatattttaatcattaaccgtgccgaagcgggcaaattggaatttaatcctaattggttagatttgaaattattgttccagcaatttatcgaagaaattcaattaagtgtcagtgaccaatattattttgactttatttgtagcgctcaagatacgaaggcattggtggatgaaaggttagtgcggtctattttatctaatctgttatctaatgcgattaaatactctcccgggggagggcagattaaaattgccctaagcctagattcggaacagattatttttgaagtcaccgaccagggcattggcatttcgccagaggaccaaaagcaaatttttgaaccctttcatcggggcaaaaatgtcagaaatattacgggaacaggactcggtttaatggttgccaagaaatgtgttgacttacacagtggcagtatcttgctaaaaagtgcagttgaccagggaacaacagttactatctgtttaaaacgctataaccatttgcctcgagcttag

SEQ ID NO: 7 - protein, 742 aa, Artificial Sequence
  MNLS CcaS (23 A92V)
MLQPKKKRKVGGRQNQERRRIEISIKQQTQRERFINQITQHIRQSLNLETVLNTTVAEVKTLLQVDRVLIYRIWQDGTGSVITESVNANYPSILGRTFSDEVFPVEYHQAYTKGKVRAINDIDQDDIEICLADFVKQFGVKSKLVVPILQHNRASSLDNESEFPYLWGLLITHQCAFTRPWQPWEVELMKQLANQVAIAIQQSELYEQLQQLNKDLENRVEKRTQQLAATNQSLRMEISERQKTEAALRHTNHTLQSLIAASPRGIFTLNLADQIQIWNPTAERIFGWTETEIIAHPELLTSNILLEDYQQFKQKVLSGMVSPSLELKCQKKDGSWIEIVLSAAPLLDSEENIAGLVAVVADITEQKRQAEQIRLLQSVVVNTNDAVVITEAEPIDDPGPRILYVNEAFTKITGYTAEEMLGKTPRVLQGPKTSRTELDRVRQAISQWQSVTVEVINYRKDGSEFWVEFSLVPVANKTGFYTHWIAVQRDVTERRRTEEVRLALEREKELSRLKTRFFSMASHEFRTPLSTALAAAQLLENSEVAWLDPDKRSRNLHRIQNSVKNMVQLLDDILIINRAEAGKLEFNPNWLDLKLLFQQFIEEIQLSVSDQYYFDFICSAQDTKALVDERLVRSILSNLLSNAIKYSPGGGQIKIALSLDSEQIIFEVTDQGIGISPEDQKQIFEPFHRGKNVRNITGTGLGLMVAKKCVDLHSGSILLKSAVDQGTTVTICLKRYNHLPRA

SEQ ID NO: 8 - DNA, 2229 bp, Artificial Sequence
  MNLS CcaS (23 A92V)
atgttacaaccaaagaagaaaaggaaggtgggtggaagacaaaaccaagaacgccgcaggattgaaattagcatcaagcaacaaacccaacgggaacgatttattaaccaaattacccaacatatccgccaatctttaaacttggaaacggttttaaataccaccgtcgctgaagttaaaaccctgttgcaagttgatcgagttctaatttatcgcatttggcaagatggcacgggcagcgtcattacggaatcggtgaatgccaattatcctagtattttagggcggaccttttccgatgaagtttttcccgttgaataccatcaagcctacaccaaaggtaaagtacgggccattaatgacattgaccaggatgacatagagatttgcctagctgatttcgtcaaacaatttggcgtgaaatcaaaattagtagtgcccattcttcaacataatcgtgcttcttccctagataatgaatcagaatttccctatctttgggggctgttaattacccatcaatgtgcttttacccggccatggcaaccgtgggaagtggagttaatgaaacagctagccaatcaggtcgcgatcgccatccaacaatcggaattatatgagcaattacagcaactcaataaagatttggaaaaccgagtcgaaaaacgcacccagcaacttgccgccaccaatcaatccctaagaatggaaatcagtgagcgacaaaaaacggaagccgctctccgccacactaaccatactctgcaatccctgattgcggcctcccccaggggtatttttacccttaatttagcagaccaaattcagatttggaatcctacagcagaacgtatttttggttggacagaaacagaaattattgcccatccagaattattaacatccaacattttgctggaagattatcagcaatttaaacagaaagttttatcaggcatggtttcccctagcctagaattaaaatgtcaaaaaaaagatggtagttggattgaaattgtcctttccgctgctcccctattggatagtgaagaaaatattgccggattggtggcggttgtcgccgatattaccgagcaaaagcggcaggcagaacaaattcgtttgctacaatccgttgtggttaatactaatgatgcggtggtgattacggaagcggagcccattgatgatcccgggccgagaattctctatgtcaatgaagcatttactaaaatcaccggttatactgctgaagaaatgctaggcaaaaccccccgagttttacagggaccaaaaactagtcgcactgaattagatagggtgcggcaagccattagtcaatggcaatcagttaccgttgaagtgattaattatcgtaaggatggcagtgagttttgggtggaatttagtctggtgcccgttgccaataaaacaggtttttacacccattggattgctgtgcaaagggatgtcactgagcgccgacgcacggaggaagtccgcctagctttagaacgggaaaaagaattaagccgcctaaaaactcgttttttctccatggcttcccatgaatttcgtactcccctcagtacggccttagctgctgcccaattactggaaaattctgaagtggcctggcttgatcccgataagcgtagccggaacttacaccgtattcaaaattccgtgaaaaatatggtacagctcctggatgatattttaatcattaaccgtgccgaagcgggcaaattggaatttaatcctaattggttagatttgaaattattgttccagcaatttatcgaagaaattcaattaagtgtcagtgaccaatattattttgactttatttgtagcgctcaagatacgaaggcattggtggatgaaaggttagtgcggtctattttatctaatctgttatctaatgcgattaaatactctcccgggggagggcagattaaaattgccctaagcctagattcggaacagattatttttgaagtcaccgaccagggcattggcatttcgccagaggaccaaaagcaaatttttgaaccctttcatcggggcaaaaatgtcagaaatattacgggaacaggactcggtttaatggttgccaagaaatgtgttgacttacacagtggcagtatcttgctaaaaagtgcagttgaccagggaacaacagttactatctgtttaaaacgctataaccatttgcctcgagcttag

SEQ ID NO: 9 - protein, 743 aa, Artificial Sequence
  MMNLS CcaS (23) F2A30 (aa1-29)
MMLQPKKKRKVGGRQNQERRRIEISIKQQTQRERFINQITQHIRQSLNLETVLNTTVAEVKTLLQVDRVLIYRIWQDGTGSAITESVNANYPSILGRTFSDEVFPVEYHQAYTKGKVRAINDIDQDDIEICLADFVKQFGVKSKLVVPILQHNRASSLDNESEFPYLWGLLITHQCAFTRPWQPWEVELMKQLANQVAIAIQQSELYEQLQQLNKDLENRVEKRTQQLAATNQSLRMEISERQKTEAALRHTNHTLQSLIAASPRGIFTLNLADQIQIWNPTAERIFGWTETEIIAHPELLTSNILLEDYQQFKQKVLSGMVSPSLELKCQKKDGSWIEIVLSAAPLLDSEENIAGLVAVVADITEQKRQAEQIRLLQSVVVNTNDAVVITEAEPIDDPGPRILYVNEAFTKITGYTAEEMLGKTPRVLQGPKTSRTELDRVRQAISQWQSVTVEVINYRKDGSEFWVEFSLVPVANKTGFYTHWIAVQRDVTERRRTEEVRLALEREKELSRLKTRFFSMASHEFRTPLSTALAAAQLLENSEVAWLDPDKRSRNLHRIQNSVKNMVQLLDDILIINRAEAGKLEFNPNWLDLKLLFQQFIEEIQLSVSDQYYFDFICSAQDTKALVDERLVRSILSNLLSNAIKYSPGGGQIKIALSLDSEQIIFEVTDQGIGISPEDQKQIFEPFHRGKNVRNITGTGLGLMVAKKCVDLHSGSILLKSAVDQGTTVTICLKRYNHLPRA

SEQ ID NO: 10 - DNA, 2229 bp, Artificial Sequence
  MMNLS CcaS (23) F2A30 (aa1-29)
atgatgttacaaccaaagaagaaaaggaaggtgggtggaagacagaaccaagaacgaagaagaatagaaataagtatcaagcagcagacacaacgtgagaggtttatcaaccaaatcacacagcatatcagacaatctcttaatttggagactgttttgaacactacagttgctgaagttaagacacttttgcaggttgatagagttcttatctatagaatctggcaagatggtacaggatctgctatcactgagtctgttaatgctaactacccttctattttgggtagaactttttctgatgaggttttcccagttgaatatcatcaagcttacacaaagggaaaagttagagctattaatgatatcgatcaggatgatatcgaaatctgtcttgctgatttcgttaaacaattcggtgttaagtctaaacttgttgttcctatcttgcagcataatagagcttcttctttggataacgaatctgagtttccatatctttggggacttttgattacacatcagtgtgctttcactagaccttggcaaccttgggaagttgagcttatgaagcagttggctaaccaagttgctattgctatccaacagtctgagttgtacgaacaacttcaacagttgaataaggatcttgagaacagagttgaaaaaagaacacaacagttggctgctactaatcagtctcttaggatggaaatctctgaaagacaaaagactgaggctgctttgagacatactaaccatacacttcagtctttgattgctgcttctcctagaggtatctttactcttaatttggctgatcaaattcagatctggaacccaacagctgagcgaatcttcggatggactgaaacagagattatcgctcatcctgagcttttgacatctaacatccttttggaagattaccaacagtttaagcaaaaggttctttctggtatggtttctccatctcttgagttgaagtgtcagaagaaagatggatcttggattgaaatcgttttgtctgctgctcctcttttggattctgaagagaacattgctggtcttgttgctgttgttgctgatatcactgagcaaaaaagacaggctgaacaaatcagacttttgcaatctgttgttgttaacacaaacgatgctgttgttattactgaagctgaaccaatcgatgatcctggaccaagaatcctttatgttaatgaggctttcactaagatcacaggatacactgctgaagagatgttgggaaagactcctagagttcttcaaggaccaaaaacttcaagaactgagttggatagagttagacaggctatctctcaatggcagtctgttacagttgaagttattaattacagaaaggatggttctgagttttgggttgaattttctcttgttcctgttgctaacaaaacaggattttacactcattggattgctgttcaaagagatgttacagagagaagaagaactgaagaggttagacttgctttggaaagagagaaggaactttcaagattgaagactagatttttctctatggcttctcatgagtttagaacaccactttctactgctttggctgctgctcaacttcttgaaaattctgaagttgcttggcttgatcctgataagagatcaagaaaccttcatagaatccaaaattctgttaaaaacatggttcaacttttggatgatatcttgattatcaacagagctgaggctggaaagcttgagtttaatccaaactggcttgatttgaagcttttgttccaacagttcattgaagagatccagctttctgtttctgatcaatactacttcgatttcatctgttctgctcaagatactaaggctcttgttgatgaaagattggttagatctatcctttctaatcttttgtctaacgctatcaagtactctcctggaggtggacagattaaaatcgctctttctttggattctgagcagattatcttcgaagttacagatcaaggtattggaatctctcctgaggatcaaaagcagatctttgaaccattccatagaggaaagaatgttagaaacattactggtacaggacttggtttgatggttgctaagaaatgtgttgatcttcattctggatctatccttttgaagtctgctgtggatcaaggaacaactgtgaccatctgtctcaaaaggtacaaccatctcccaagggct

SEQ ID NO: 11 - protein, 743 aa, Artificial Sequence
  MMNLS CcaS (23 A92V) F2A30 (aa1-29)
MMLQPKKKRKVGGRQNQERRRIEISIKQQTQRERFINQITQHIRQSLNLETVLNTTVAEVKTLLQVDRVLIYRIWQDGTGSVITESVNANYPSILGRTFSDEVFPVEYHQAYTKGKVRAINDIDQDDIEICLADFVKQFGVKSKLVVPILQHNRASSLDNESEFPYLWGLLITHQCAFTRPWQPWEVELMKQLANQVAIAIQQSELYEQLQQLNKDLENRVEKRTQQLAATNQSLRMEISERQKTEAALRHTNHTLQSLIAASPRGIFTLNLADQIQIWNPTAERIFGWTETEIIAHPELLTSNILLEDYQQFKQKVLSGMVSPSLELKCQKKDGSWIEIVLSAAPLLDSEENIAGLVAVVADITEQKRQAEQIRLLQSVVVNTNDAVVITEAEPIDDPGPRILYVNEAFTKITGYTAEEMLGKTPRVLQGPKTSRTELDRVRQAISQWQSVTVEVINYRKDGSEFWVEFSLVPVANKTGFYTHWIAVQRDVTERRRTEEVRLALEREKELSRLKTRFFSMASHEFRTPLSTALAAAQLLENSEVAWLDPDKRSRNLHRIQNSVKNMVQLLDDILIINRAEAGKLEFNPNWLDLKLLFQQFIEEIQLSVSDQYYFDFICSAQDTKALVDERLVRSILSNLLSNAIKYSPGGGQIKIALSLDSEQIIFEVTDQGIGISPEDQKQIFEPFHRGKNVRNITGTGLGLMVAKKCVDLHSGSILLKSAVDQGTTVTICLKRYNHLPRA

SEQ ID NO: 12 - DNA, 2229 bp, Artificial Sequence
  MMNLS CcaS (23 A92V) F2A30 (aa1-29)
atgatgttacaaccaaagaagaaaaggaaggtgggtggaagacagaaccaagaacgaagaagaatagaaataagtatcaagcagcagacacaacgtgagaggtttatcaaccaaatcacacagcatatcagacaatctcttaatttggagactgttttgaacactacagttgctgaagttaagacacttttgcaggttgatagagttcttatctatagaatctggcaagatggtacaggatctgttatcactgagtctgttaatgctaactacccttctattttgggtagaactttttctgatgaggttttcccagttgaatatcatcaagcttacacaaagggaaaagttagagctattaatgatatcgatcaggatgatatcgaaatctgtcttgctgatttcgttaaacaattcggtgttaagtctaaacttgttgttcctatcttgcagcataatagagcttcttctttggataacgaatctgagtttccatatctttggggacttttgattacacatcagtgtgctttcactagaccttggcaaccttgggaagttgagcttatgaagcagttggctaaccaagttgctattgctatccaacagtctgagttgtacgaacaacttcaacagttgaataaggatcttgagaacagagttgaaaaaagaacacaacagttggctgctactaatcagtctcttaggatggaaatctctgaaagacaaaagactgaggctgctttgagacatactaaccatacacttcagtctttgattgctgcttctcctagaggtatctttactcttaatttggctgatcaaattcagatctggaacccaacagctgagcgaatcttcggatggactgaaacagagattatcgctcatcctgagcttttgacatctaacatccttttggaagattaccaacagtttaagcaaaaggttctttctggtatggtttctccatctcttgagttgaagtgtcagaagaaagatggatcttggattgaaatcgttttgtctgctgctcctcttttggattctgaagagaacattgctggtcttgttgctgttgttgctgatatcactgagcaaaaaagacaggctgaacaaatcagacttttgcaatctgttgttgttaacacaaacgatgctgttgttattactgaagctgaaccaatcgatgatcctggaccaagaatcctttatgttaatgaggctttcactaagatcacaggatacactgctgaagagatgttgggaaagactcctagagttcttcaaggaccaaaaacttcaagaactgagttggatagagttagacaggctatctctcaatggcagtctgttacagttgaagttattaattacagaaaggatggttctgagttttgggttgaattttctcttgttcctgttgctaacaaaacaggattttacactcattggattgctgttcaaagagatgttacagagagaagaagaactgaagaggttagacttgctttggaaagagagaaggaactttcaagattgaagactagatttttctctatggcttctcatgagtttagaacaccactttctactgctttggctgctgctcaacttcttgaaaattctgaagttgcttggcttgatcctgataagagatcaagaaaccttcatagaatccaaaattctgttaaaaacatggttcaacttttggatgatatcttgattatcaacagagctgaggctggaaagcttgagtttaatccaaactggcttgatttgaagcttttgttccaacagttcattgaagagatccagctttctgtttctgatcaatactacttcgatttcatctgttctgctcaagatactaaggctcttgttgatgaaagattggttagatctatcctttctaatcttttgtctaacgctatcaagtactctcctggaggtggacagattaaaatcgctctttctttggattctgagcagattatcttcgaagttacagatcaaggtattggaatctctcctgaggatcaaaagcagatctttgaaccattccatagaggaaagaatgttagaaacattactggtacaggacttggtttgatggttgctaagaaatgtgttgatcttcattctggatctatccttttgaagtctgctgtggatcaaggaacaactgtgaccatctgtctcaaaaggtacaaccatctcccaagggct

SEQ ID NO: 13 - protein, 316 aa, Artificial Sequence
  F2A30(aa30)NLS2xGGSVP644xGGSCcaR
PGSLQPKKKRKVGGGGSGGSDALDDFDLDMLGSDALDDFDLDMLGSDALDDFDLDMLGSDALDDFDLDMLGGSGGSGGSGGSMRILLVEDDLPLAETLAEALSDQLYTVDIATDASLAWDYASRLEYDLVILDVMLPELDGITLCQKWRSHSYLMPILMMTARDTINDKITGLDAGADDYVVKPVDLGELFARVRALLRRGCATCQPVLEWGPIRLDPSTYEVSYDNEVLSLTRKEYSILELLLRNGRRVLSRSMIIDSIWKLESPPEEDTVKVHVRSLRQKLKSAGLSADAIETVHGIGYRLANLTEKSLCQGKN

SEQ ID NO: 14 - DNA, 948 bp, Artificial Sequence
  F2A30(aa30)NLS2xGGSVP644xGGSCcaR
ccaggttcactccagcctaagaagaagagaaaggttggaggtggtggctccggaggctctgatgccctcgacgatttcgacctcgatatgctcggttctgatgctctcgatgactttgaccttgacatgcttggatcagacgctttggacgacttcgacttggacatgttgggatctgatgcacttgatgattttgaccttgatatgcttggtggttcaggagggtctggtggatcaggaggatctatgagaatactcctcgtggaagatgatttgccattagcagaaaccctcgcagaagctttgtctgatcaactttacactgttgatattgctacagatgcttctttggcttgggattatgcttctagacttgaatacgatttggttattcttgatgttatgttgcctgagcttgatggaattactctttgtcagaagtggagatctcattcttatttgatgccaatccttatgatgactgctagagatacaattaatgataagatcacaggacttgatgctggtgctgatgattacgttgttaaacctgttgatttgggtgaactttttgctagagttagagctcttttgagaagaggatgtgctacttgtcaaccagttttggagtggggtcctattagacttgatccatctacttatgaagtttcttacgataatgaggttttgtctcttacaagaaaggaatactctatcttggagcttttgcttagaaacggaagaagagttctttctagatctatgatcatcgattctatctggaagttggagtctcctccagaagaggatacagttaaagttcatgttagatctttgagacaaaagcttaagtctgctggactttctgctgatgctattgaaactgttcatggaatcggttacagattggctaatcttacagagaagtctttgtgtcagggaaagaat

SEQ ID NO: 15 - protein, 314 aa, Artificial Sequence
  F2A30(aa30)CcaR4xGSSVP642xGGSNLS
PMRILLVEDDLPLAETLAEALSDQLYTVDIATDASLAWDYASRLEYDLVILDVMLPELDGITLCQKWRSHSYLMPILMMTARDTINDKITGLDAGADDYVVKPVDLGELFARVRALLRRGCATCQPVLEWGPIRLDPSTYEVSYDNEVLSLTRKEYSILELLLRNGRRVLSRSMIIDSIWKLESPPEEDTVKVHVRSLRQKLKSAGLSADAIETVHGIGYRLANLTEKSLCQGKNGGSGGSGGSGGSDALDDFDLDMLGSDALDDFDLDMLGSDALDDFDLDMLGSDALDDFDLDMLGGSGGSLQPKKKRKVGG

SEQ ID NO: 16 - DNA, 942 bp, Artificial Sequence
  F2A30(aa30)CcaR4xGSSVP642xGGSNLS
ccaatgagaatactcctcgtggaagatgatttgccattagcagaaaccctcgcagaagctttgtctgatcaactttacactgttgatattgctacagatgcttctttggcttgggattatgcttctagacttgaatacgatttggttattcttgatgttatgttgcctgagcttgatggaattactctttgtcagaagtggagatctcattcttatttgatgccaatccttatgatgactgctagagatacaattaatgataagatcacaggacttgatgctggtgctgatgattacgttgttaaacctgttgatttgggtgaactttttgctagagttagagctcttttgagaagaggatgtgctacttgtcaaccagttttggagtggggtcctattagacttgatccatctacttatgaagtttcttacgataatgaggttttgtctcttacaagaaaggaatactctatcttggagcttttgcttagaaacggaagaagagttctttctagatctatgatcatcgattctatctggaagttggagtctcctccagaagaggatacagttaaagttcatgttagatctttgagacaaaagcttaagtctgctggactttctgctgatgctattgaaactgttcatggaatcggttacagattggctaatcttacagagaagtctttgtgtcagggaaagaatggaggctccggtgggtcaggtggttctggaggctcggatgccctcgacgatttcgacctcgatatgctcggttctgatgctctcgatgactttgaccttgacatgcttggatcagacgctttggacgacttcgacttggacatgttgggatctgatgcacttgatgattttgaccttgatatgcttggcggttccggtggatcactccagcctaagaagaagagaaaggttggaggt

SEQ ID NO: 17 - DNA, 125 bp, Artificial Sequence
  promoter
ctttccgatttctttacgatttccgctttccgatttctttacgatttggctttccgatttctttacgatttatccttcgcaagacccttcctctatataaggaagttcatttcatttggagagga

SEQ ID NO: 18 - DNA, 477 bp, Artificial Sequence
  GAF domain
atcagacaatctcttaatttggagactgttttgaacactacagttgctgaagttaagacacttttgcaggttgatagagttcttatctatagaatctggcaagatggtacaggatctgttatcactgagtctgttaatgctaactacccttctattttgggtagaactttttctgatgaggttttcccagttgaatatcatcaagcttacacaaagggaaaagttagagctattaatgatatcgatcaggatgatatcgaaatctgtcttgctgatttcgttaaacaattcggtgttaagtctaaacttgttgttcctatcttgcagcataatagagcttcttctttggataacgaatctgagtttccatatctttggggacttttgattacacatcagtgtgctttcactagaccttggcaaccttgggaagttgagcttatgaagcagttggctaaccaagttgctattgctatc

SEQ ID NO: 19 - protein, 159 aa, Artificial Sequence
  GAF domain
IRQSLNLETVLNTTVAEVKTLLQVDRVLIYRIWQDGTGSVITESVNANYPSILGRTFSDEVFPVEYHQAYTKGKVRAINDIDQDDIEICLADFVKQFGVKSKLVVPILQHNRASSLDNESEFPYLWGLLITHQCAFTRPWQPWEVELMKQLANQVAIAI

SEQ ID NO: 20 - DNA, 222 bp, Artificial Sequence
  PAS domain 1
actaaccatacacttcagtctttgattgctgcttctcctagaggtatctttactcttaatttggctgatcaaattcagatctggaacccaacagctgagcgaatcttcggatggactgaaacagagattatcgctcatcctgagcttttgacatctaacatccttttggaagattaccaacagtttaagcaaaaggttctttctggtatggtttctccatct

SEQ ID NO: 21 - protein, 74 aa, Artificial Sequence
  PAS domain 1
TNHTLQSLIAASPRGIFTLNLADQIQIWNPTAERIFGWTETEIIAHPELLTSNILLEDYQQFKQKVLSGMVSPS

SEQ ID NO: 22 - DNA, 162 bp, Artificial Sequence
  PAS domain 2
atcgatgatcctggaccaagaatcctttatgttaatgaggctttcactaagatcacaggatacactgctgaagagatgttgggaaagactcctagagttcttcaaggaccaaaaacttcaagaactgagttggatagagttagacaggctatctctcaatgg

SEQ ID NO: 23 - protein, 54 aa, Artificial Sequence
  PAS domain 2
IDDPGPRILYVNEAFTKITGYTAEEMLGKTPRVLQGPKTSRTELDRVRQAISQW

SEQ ID NO: 24 - DNA, 654 bp, Artificial Sequence
  His-Kinase domain
atggcttctcatgagtttagaacaccactttctactgctttggctgctgctcaacttcttgaaaattctgaagttgcttggcttgatcctgataagagatcaagaaaccttcatagaatccaaaattctgttaaaaacatggttcaacttttggatgatatcttgattatcaacagagctgaggctggaaagcttgagtttaatccaaactggcttgatttgaagcttttgttccaacagttcattgaagagatccagctttctgtttctgatcaatactacttcgatttcatctgttctgctcaagatactaaggctcttgttgatgaaagattggttagatctatcctttctaatcttttgtctaacgctatcaagtactctcctggaggtggacagattaaaatcgctctttctttggattctgagcagattatcttcgaagttacagatcaaggtattggaatctctcctgaggatcaaaagcagatctttgaaccattccatagaggaaagaatgttagaaacattactggtacaggacttggtttgatggttgctaagaaatgtgttgatcttcattctggatctatccttttgaagtctgctgtggatcaaggaacaactgtgaccatctgtctcaaaaggtacaac

SEQ ID NO: 25 - protein, 218 aa, Artificial Sequence
  His-Kinase domain
MASHEFRTPLSTALAAAQLLENSEVAWLDPDKRSRNLHRIQNSVKNMVQLLDDILIINRAEAGKLEFNPNWLDLKLLFQQFIEEIQLSVSDQYYFDFICSAQDTKALVDERLVRSILSNLLSNAIKYSPGGGQIKIALSLDSEQIIFEVTDQGIGISPEDQKQIFEPFHRGKNVRNITGTGLGLMVAKKCVDLHSGSILLKSAVDQGTTVTICLKRYN

SEQ ID NO: 26 - DNA, 33 bp, Artificial Sequence
  NLS
ttacaaccaaagaagaaaaggaaggtgggtgga

SEQ ID NO: 27 - protein, 11 aa, Artificial Sequence
  NLS
LQPKKKRKVGG

SEQ ID NO: 28 - DNA, 345 bp, Artificial Sequence
  REC domain
agaatactcctcgtggaagatgatttgccattagcagaaaccctcgcagaagctttgtctgatcaactttacactgttgatattgctacagatgcttctttggcttgggattatgcttctagacttgaatacgatttggttattcttgatgttatgttgcctgagcttgatggaattactctttgtcagaagtggagatctcattcttatttgatgccaatccttatgatgactgctagagatacaattaatgataagatcacaggacttgatgctggtgctgatgattacgttgttaaacctgttgatttgggtgaactttttgctagagttagagctcttttg

SEQ ID NO: 29 - protein, 115 aa, Artificial Sequence
  REC domain
RILLVEDDLPLAETLAEALSDQLYTVDIATDASLAWDYASRLEYDLVILDVMLPELDGITLCQKWRSHSYLMPILMMTARDTINDKITGLDAGADDYVVKPVDLGELFARVRALL

SEQ ID NO: 30 - DNA, 300 bp, Artificial Sequence
  DNA binding domain
caaccagttttggagtggggtcctattagacttgatccatctacttatgaagtttcttacgataatgaggttttgtctcttacaagaaaggaatactctatcttggagcttttgcttagaaacggaagaagagttctttctagatctatgatcatcgattctatctggaagttggagtctcctccagaagaggatacagttaaagttcatgttagatctttgagacaaaagcttaagtctgctggactttctgctgatgctattgaaactgttcatggaatcggttacagattggctaat

SEQ ID NO: 31 - protein, 100 aa, Artificial Sequence
  DNA binding domain
QPVLEWGPIRLDPSTYEVSYDNEVLSLTRKEYSILELLLRNGRRVLSRSMIIDSIWKLESPPEEDTVKVHVRSLRQKLKSAGLSADAIETVHGIGYRLAN

SEQ ID NO: 32 - DNA, 33 bp, Artificial Sequence
  NLS
ctccagcctaagaagaagagaaaggttggaggt

SEQ ID NO: 33 - protein, 11 aa, Artificial Sequence
  NLS
LQPKKKRKVGG

SEQ ID NO: 34 - DNA, 150 bp, Artificial Sequence
  VP64 domain
gatgccctcgacgatttcgacctcgatatgctcggttctgatgctctcgatgactttgaccttgacatgcttggatcagacgctttggacgacttcgacttggacatgttgggatctgatgcacttgatgattttgaccttgatatgctt

SEQ ID NO: 35 - protein, 50 aa, Artificial Sequence
  VP64 domain
DALDDFDLDMLGSDALDDFDLDMLGSDALDDFDLDMLGSDALDDFDLDML

SEQ ID NO: 36 - DNA, 63 bp, Artificial Sequence
  F2A
ggacaacttctcaactttgacttgctaaagttagctggtgatgttgaatctaatcctggacca

SEQ ID NO: 37 - protein, 20 aa, Artificial Sequence
  F2Aaa1-20
GQLLNFDLLKLAGDVESNPG

SEQ ID NO: 38 - DNA, 90 bp, Artificial Sequence
  F2A30
cacaaacagaaaattgtggcaccggtgaagcagactctcaactttgacttgctaaagttagctggtgatgttgaatctaatcctggacca

SEQ ID NO: 39 - protein, 29 aa, Artificial Sequence
  F2Aaa1-20
HKQKIVAPVKQTLNFDLLKLAGDVESNPG

SEQ ID NO: 40 - DNA, 22 bp, Artificial Sequence
  ccaR CRE motif
ctttccgatttctttacgattt

SEQ ID NO: 41 - DNA, 51 bp, Artificial Sequence
  P35Smin(-51)
cttcgcaagacccttcctctatataaggaagttcatttcatttggagagga

SEQ ID NO: 42 - DNA, 645 bp, Artificial Sequence
  Terminator sequence (Trbcs)
agctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcg

SEQ ID NO: 43 - DNA, 472 bp, Artificial Sequence
  Terminator sequence (NOS terminator)
tagagtagatgccgaccgaacaagagctgatttcgagaacgcctcagccagcaactcgcgcgagcctagcaaggcaaatgcgagagaacggccttacgcttggtggcacagttctcgtccacagttcgctaagctcgctcggctgggtcgcgggagggccggtcgcagtgattcaggaattaattccctagagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaccggcatgcaagctgat

SEQ ID NO: 44 - DNA, 652 bp, Artificial Sequence
  UBQ10 promoter
acccgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtagattaatctgagtttttctgattaacagctcgagtgcgggatc

SEQ ID NO: 45 - DNA, 74 bp, Artificial Sequence
  PRR
caggctgcgcaactggctttccgatttctttacgatttccgctttccgatttctttacgatttggctttccgat

SEQ ID NO: 46 - DNA, 74 bp, Artificial Sequence
  PRR
ttctttacgatttatccttcgcaagacccttcctctatataaggaagttcatttcatttggagaggacacgctg

SEQ ID NO: 47 - DNA, 2292 bp, Artificial Sequence
  LRHK1-01
atgatgttacaaccaaagaagaaaaggaaggtgggtggaagacaaaaccaagaacgccgcaggattgaaattagcatcaagcaacaaacccaacgggaacgatttattaaccaaattacccaacatatccgccaatctttaaacttggaaacggttttaaataccaccgtcgctgaagttaaaaccctgttgcaagttgatcgagttgccgtgtaccgttttaacccggattggagcggcgagtttgtggccgaaagcgtgggtagcggttgggtgaaactggtgggcccggatatcaaaaccgtgtgggaagacacacatctgcaagaaacccaaggtggtcgctatcgccatcaagaaagcttcgtggtgaacgacatttatgaggccggccatttcagctgccatctggagattttagaacagtttgaaattaaagcctacattatcgtgccggtttttgccgccgaaaaactgtggggtttactggccgcctatcagaacagtggtacccgcgaatgggtggaatgggaaagcagctttctgacccaagttggtctgcagttcggcatcgccatccaacaatcggaattatatgagcaattacagcaactcaataaagatttggaaaaccgagtcgaaaaacgcacccagcaacttgccgccaccaatcaatccctaagaatggaaatcagtgagcgacaaaaaacggaagccgctctccgccacactaaccatactctgcaatccctgattgcggcctcccccaggggtatttttacccttaatttagcagaccaaattcagatttggaatcctacagcagaacgtatttttggttggacagaaacagaaattattgcccatccagaattattaacatccaacattttgctggaagattatcagcaatttaaacagaaagttttatcaggcatggtttcccctagcctagaattaaaatgtcaaaaaaaagatggtagttggattgaaattgtcctttccgctgctcccctattggatagtgaagaaaatattgccggattggtggcggttgtcgccgatattaccgagcaaaagcggcaggcagaacaaattcgtttgctacaatccgttgtggttaatactaatgatgcggtggtgattacggaagcggagcccattgatgatcccgggccgagaattctctatgtcaatgaagcatttactaaaatcaccggttatactgctgaagaaatgctaggcaaaaccccccgagttttacagggaccaaaaactagtcgcactgaattagatagggtgcggcaagccattagtcaatggcaatcagttaccgttgaagtgattaattatcgtaaggatggcagtgagttttgggtggaatttagtctggtgcccgttgccaataaaacaggtttttacacccattggattgctgtgcaaagggatgtcactgagcgccgacgcacggaggaagtccgcctagctttagaacgggaaaaagaattaagccgcctaaaaactcgttttttctccatggcttcccatgaatttcgtactcccctcagtacggccttagctgctgcccaattactggaaaattctgaagtggcctggcttgatcccgataagcgtagccggaacttacaccgtattcaaaattccgtgaaaaatatggtacagctcctggatgatattttaatcattaaccgtgccgaagcgggcaaattggaatttaatcctaattggttagatttgaaattattgttccagcaatttatcgaagaaattcaattaagtgtcagtgaccaatattattttgactttatttgtagcgctcaagatacgaaggcattggtggatgaaaggttagtgcggtctattttatctaatctgttatctaatgcgattaaatactctcccgggggagggcagattaaaattgccctaagcctagattcggaacagattatttttgaagtcaccgaccagggcattggcatttcgccagaggaccaaaagcaaatttttgaaccctttcatcggggcaaaaatgtcagaaatattacgggaacaggactcggtttaatggttgccaagaaatgtgttgacttacacagtggcagtatcttgctaaaaagtgcagttgaccagggaacaacagttactatctgtttaaaacgctataaccatttgcctcgagctcacaaacagaaaattgtggcaccggtgaagcagactctcaactttgacttgctaaagttagctggtgatgttgaatctaatcctgga

SEQ ID NO: 48 - DNA, 2295 bp, Artificial Sequence
  LRHK1-05
atgatgttacaaccaaagaagaaaaggaaggtgggtggaagacaaaaccaagaacgccgcaggattgaaattagcatcaagcaacaaacccaacgggaacgatttattaaccaaattacccaacatatccgccaatctttaaacttggaaacggttttaaataccaccgtcgctgaagttaaaaccctgttgcaagttgatcgagttctggtgtatcgctttaacccggattggagcggcgagtttatccatgaaagcgtggcccagatgtgggaaccgctgaaggatctgcagaacaactttccgctgtggcaagatacctatttacaagaaaatgagggtggccgctaccgcaatcatgaaagtctggccgtgggcgatgtggaaaccgccggtttcaccgattgccatttagataatctgcgtcgcttcgaaattcgcgcctttctgaccgtgccggtttttgttggtgaacagctgtggggtctgctgggcgcctatcagaatggtgcaccgcgccattggcaagctcgcgaaattcatctgctgcaccagatcgccaaccagctgggtatcgccatccaacaatcggaattatatgagcaattacagcaactcaataaagatttggaaaaccgagtcgaaaaacgcacccagcaacttgccgccaccaatcaatccctaagaatggaaatcagtgagcgacaaaaaacggaagccgctctccgccacactaaccatactctgcaatccctgattgcggcctcccccaggggtatttttacccttaatttagcagaccaaattcagatttggaatcctacagcagaacgtatttttggttggacagaaacagaaattattgcccatccagaattattaacatccaacattttgctggaagattatcagcaatttaaacagaaagttttatcaggcatggtttcccctagcctagaattaaaatgtcaaaaaaaagatggtagttggattgaaattgtcctttccgctgctcccctattggatagtgaagaaaatattgccggattggtggcggttgtcgccgatattaccgagcaaaagcggcaggcagaacaaattcgtttgctacaatccgttgtggttaatactaatgatgcggtggtgattacggaagcggagcccattgatgatcccgggccgagaattctctatgtcaatgaagcatttactaaaatcaccggttatactgctgaagaaatgctaggcaaaaccccccgagttttacagggaccaaaaactagtcgcactgaattagatagggtgcggcaagccattagtcaatggcaatcagttaccgttgaagtgattaattatcgtaaggatggcagtgagttttgggtggaatttagtctggtgcccgttgccaataaaacaggtttttacacccattggattgctgtgcaaagggatgtcactgagcgccgacgcacggaggaagtccgcctagctttagaacgggaaaaagaattaagccgcctaaaaactcgttttttctccatggcttcccatgaatttcgtactcccctcagtacggccttagctgctgcccaattactggaaaattctgaagtggcctggcttgatcccgataagcgtagccggaacttacaccgtattcaaaattccgtgaaaaatatggtacagctcctggatgatattttaatcattaaccgtgccgaagcgggcaaattggaatttaatcctaattggttagatttgaaattattgttccagcaatttatcgaagaaattcaattaagtgtcagtgaccaatattattttgactttatttgtagcgctcaagatacgaaggcattggtggatgaaaggttagtgcggtctattttatctaatctgttatctaatgcgattaaatactctcccgggggagggcagattaaaattgccctaagcctagattcggaacagattatttttgaagtcaccgaccagggcattggcatttcgccagaggaccaaaagcaaatttttgaaccctttcatcggggcaaaaatgtcagaaatattacgggaacaggactcggtttaatggttgccaagaaatgtgttgacttacacagtggcagtatcttgctaaaaagtgcagttgaccagggaacaacagttactatctgtttaaaacgctataaccatttgcctcgagctcacaaacagaaaattgtggcaccggtgaagcagactctcaactttgacttgctaaagttagctggtgatgttgaatctaatcctgga

SEQ ID NO: 49 - DNA, 2286 bp, Artificial Sequence
  LRHK1-10
atgatgttacaaccaaagaagaaaaggaaggtgggtggaagacaaaaccaagaacgccgcaggattgaaattagcatcaagcaacaaacccaacgggaacgatttattaaccaaattacccaacatatccgccaatctttaaacttggaaacggttttaaataccaccgtcgctgaagttaaaaccctgttgcaagttgatcgagttaccatttatcgttttcgcgccgattggagcggtgaatttgtggccgaatctttagcccaaggttggacaccggtgcgtgaaattgtgccggtggttgccgatgactatctgcaagaaacccaaggtcgcaactttgccaatggcaaaagcatcgtgattaaagatatttacagcgccaactacagcatctgccacattgcactgctggaactgatgcaagctcgcgcctatatgatcgtgccgatcttccaaggtgaaaagctgtggggtctgctggccgcctatcagaacatcaagcctcgcgattggcaagaagatgaggtggatctggtgatgcagatcggtacccagctgggcatcgccatccaacaatcggaattatatgagcaattacagcaactcaataaagatttggaaaaccgagtcgaaaaacgcacccagcaacttgccgccaccaatcaatccctaagaatggaaatcagtgagcgacaaaaaacggaagccgctctccgccacactaaccatactctgcaatccctgattgcggcctcccccaggggtatttttacccttaatttagcagaccaaattcagatttggaatcctacagcagaacgtatttttggttggacagaaacagaaattattgcccatccagaattattaacatccaacattttgctggaagattatcagcaatttaaacagaaagttttatcaggcatggtttcccctagcctagaattaaaatgtcaaaaaaaagatggtagttggattgaaattgtcctttccgctgctcccctattggatagtgaagaaaatattgccggattggtggcggttgtcgccgatattaccgagcaaaagcggcaggcagaacaaattcgtttgctacaatccgttgtggttaatactaatgatgcggtggtgattacggaagcggagcccattgatgatcccgggccgagaattctctatgtcaatgaagcatttactaaaatcaccggttatactgctgaagaaatgctaggcaaaaccccccgagttttacagggaccaaaaactagtcgcactgaattagatagggtgcggcaagccattagtcaatggcaatcagttaccgttgaagtgattaattatcgtaaggatggcagtgagttttgggtggaatttagtctggtgcccgttgccaataaaacaggtttttacacccattggattgctgtgcaaagggatgtcactgagcgccgacgcacggaggaagtccgcctagctttagaacgggaaaaagaattaagccgcctaaaaactcgttttttctccatggcttcccatgaatttcgtactcccctcagtacggccttagctgctgcccaattactggaaaattctgaagtggcctggcttgatcccgataagcgtagccggaacttacaccgtattcaaaattccgtgaaaaatatggtacagctcctggatgatattttaatcattaaccgtgccgaagcgggcaaattggaatttaatcctaattggttagatttgaaattattgttccagcaatttatcgaagaaattcaattaagtgtcagtgaccaatattattttgactttatttgtagcgctcaagatacgaaggcattggtggatgaaaggttagtgcggtctattttatctaatctgttatctaatgcgattaaatactctcccgggggagggcagattaaaattgccctaagcctagattcggaacagattatttttgaagtcaccgaccagggcattggcatttcgccagaggaccaaaagcaaatttttgaaccctttcatcggggcaaaaatgtcagaaatattacgggaacaggactcggtttaatggttgccaagaaatgtgttgacttacacagtggcagtatcttgctaaaaagtgcagttgaccagggaacaacagttactatctgtttaaaacgctataaccatttgcctcgagctcacaaacagaaaattgtggcaccggtgaagcagactctcaactttgacttgctaaagttagctggtgatgttgaatctaatcctgga

SEQ ID NO: 50 - DNA, 2289 bp, Artificial Sequence
  LRHK1-12
atgatgttacaaccaaagaagaaaaggaaggtgggtggaagacaaaaccaagaacgccgcaggattgaaattagcatcaagcaacaaacccaacgggaacgatttattaaccaaattacccaacatatccgccaatctttaaacttggaaacggttttaaataccaccgtcgctgaagttaaaaccctgttgcaagttgatcgagttgttatttttcagttttcacccgactctgacttttccgttggtaatattgtggcagagtcggtattggctccatttaagccaatcattaatagtgcaattgaagaaacttgttttagtaataactatgcccaaaggtatcagcagggcagaattcaggtcattgaggatattcaccagtcccatcttaggcaatgccacattgactttcttgccaggctacaggtcagggcaaacctagtgctaccactaattaatgatgccattttgtggggcttattgtgtattcatcaatgtgacagttctagagtttgggaacaaacagaaattgatctgctcaagcagatcactaatcagtttgaaatcgccatccaacaatcggaattatatgagcaattacagcaactcaataaagatttggaaaaccgagtcgaaaaacgcacccagcaacttgccgccaccaatcaatccctaagaatggaaatcagtgagcgacaaaaaacggaagccgctctccgccacactaaccatactctgcaatccctgattgcggcctcccccaggggtatttttacccttaatttagcagaccaaattcagatttggaatcctacagcagaacgtatttttggttggacagaaacagaaattattgcccatccagaattattaacatccaacattttgctggaagattatcagcaatttaaacagaaagttttatcaggcatggtttcccctagcctagaattaaaatgtcaaaaaaaagatggtagttggattgaaattgtcctttccgctgctcccctattggatagtgaagaaaatattgccggattggtggcggttgtcgccgatattaccgagcaaaagcggcaggcagaacaaattcgtttgctacaatccgttgtggttaatactaatgatgcggtggtgattacggaagcggagcccattgatgatcccgggccgagaattctctatgtcaatgaagcatttactaaaatcaccggttatactgctgaagaaatgctaggcaaaaccccccgagttttacagggaccaaaaactagtcgcactgaattagatagggtgcggcaagccattagtcaatggcaatcagttaccgttgaagtgattaattatcgtaaggatggcagtgagttttgggtggaatttagtctggtgcccgttgccaataaaacaggtttttacacccattggattgctgtgcaaagggatgtcactgagcgccgacgcacggaggaagtccgcctagctttagaacgggaaaaagaattaagccgcctaaaaactcgttttttctccatggcttcccatgaatttcgtactcccctcagtacggccttagctgctgcccaattactggaaaattctgaagtggcctggcttgatcccgataagcgtagccggaacttacaccgtattcaaaattccgtgaaaaatatggtacagctcctggatgatattttaatcattaaccgtgccgaagcgggcaaattggaatttaatcctaattggttagatttgaaattattgttccagcaatttatcgaagaaattcaattaagtgtcagtgaccaatattattttgactttatttgtagcgctcaagatacgaaggcattggtggatgaaaggttagtgcggtctattttatctaatctgttatctaatgcgattaaatactctcccgggggagggcagattaaaattgccctaagcctagattcggaacagattatttttgaagtcaccgaccagggcattggcatttcgccagaggaccaaaagcaaatttttgaaccctttcatcggggcaaaaatgtcagaaatattacgggaacaggactcggtttaatggttgccaagaaatgtgttgacttacacagtggcagtatcttgctaaaaagtgcagttgaccagggaacaacagttactatctgtttaaaacgctataaccatttgcctcgagctcacaaacagaaaattgtggcaccggtgaagcagactctcaactttgacttgctaaagttagctggtgatgttgaatctaatcctgga